SEQUENCE LISTING (10 sequences)

SEQ ID NO: 1 - DNA, 130 bp, synthetic construct
  source 1..130: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 2 - DNA, 321 bp, synthetic construct
  source 1..321: mol_type = other DNA
aggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggg

SEQ ID NO: 3 - DNA, 393 bp, synthetic construct
  source 1..393: mol_type = other DNA
gatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagt

SEQ ID NO: 4 - DNA, 133 bp, synthetic construct
  source 1..133: mol_type = other DNA
gtaagtatcaaggttacaagacaggtttaaggagaccaatagaaactgggcttgtcgagacagagaagactcttgcgtttctgataggcacctattggtcttactgacatccactttgcctttctctccacag

SEQ ID NO: 5 - DNA, 1290 bp, synthetic construct
  source 1..1290: mol_type = other DNA
atgcaacttaggaaccccgaacttcatcttggctgcgccctggccctccgcttcctcgctctcgtttcttgggacatccctggcgctagggcactcgacaacggcctcgcgcggactcctacgatgggatggttgcactgggaaaggtttatgtgcaatctggattgccaggaggagccggactcatgcatctcggagaagctgttcatggagatggcggaacttatggtatcggagggatggaaggatgccgggtatgagtatctctgtatcgacgattgttggatggctccccagagagactccgagggacgactccaagcggacccccagcgctttccacatggcattcgacagctcgccaattacgtgcactcgaaggggttgaagttgggaatctacgcagatgtgggcaacaaaacgtgtgcggggttcccggggtcgtttggatactacgatattgatgcgcagacgtttgctgactggggtgtcgatcttttgaaatttgatggctgttactgtgattcgttggaaaacctggcggatggatacaagcatatgtcactcgccttgaaccggacaggtcgctcaatcgtatacagctgcgaatggcccctctatatgtggcccttccaaaagcccaattacacagagattcggcagtattgcaatcactggaggaactttgccgatattgacgacagctggaaatccatcaagtccattctcgattggacgagcttcaaccaggagcgcatcgtggacgtggcaggacccggaggttggaacgatccggacatgctcgtaattgggaatttcgggcttagctggaatcagcaagtcacccaaatggcgctgtgggccatcatggcagctcctctctttatgtcgaatgatctgcggcatatctcgccccaggcaaaggctcttttgcaagacaaggacgtcatcgcaatcaatcaggacccattggggaaacagggatatcaacttcgccagggtgacaatttcgaagtatgggagaggccgcttagcgggctggcgtgggcggtcgcgatgattaaccggcaggaaatcggagggcctcgctcgtataccatcgcagtggcctcactgggcaaaggagtggcgtgcaatccggcctgcttcatcacccagttgttgcccgtcaaaagaaagctgggtttctacgagtggacatccagacttagatcacacattaaccctactggtacggtgttgctccagctcgaaaacacaatgcagatgtcgttgaaagacctgctgtaa

SEQ ID NO: 6 - DNA, 592 bp, synthetic construct
  source 1..592: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctg

SEQ ID NO: 7 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 8 - DNA, 108 bp, synthetic construct
  source 1..108: mol_type = other DNA
aggaacccctagtgatggagttggccactccctctctgcgcgctcgctcgctcactgaggccgcccgggctttgcccgggcggcctcagtgagcgagcgagcgcgcag

SEQ ID NO: 9 - DNA, 3321 bp, synthetic construct
  source 1..3321: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggcctagtaggctcagaggcacacaggagtttctgggctcaccctgcccccttccaacccctcagttcccatcctccagcagctgtttgtgtgctgcctctgaagtccacactgaacaaacttcagcctactcatgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggtacccggggatcttgctaccagtggaacagccactaaggattctgcagtgagagcagagggccagctaagtggtactctcccagagactgtctgactcacgccaccccctccaccttggacacaggacgctgtggtttctgagccaggtacaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggacagtcaggtaagtatcaaggttacaagacaggtttaaggagaccaatagaaactgggcttgtcgagacagagaagactcttgcgtttctgataggcacctattggtcttactgacatccactttgcctttctctccacaggcaattgatccccctgatctgcggcctcgacggtatcgataagcttgccaccatgcaacttaggaaccccgaacttcatcttggctgcgccctggccctccgcttcctcgctctcgtttcttgggacatccctggcgctagggcactcgacaacggcctcgcgcggactcctacgatgggatggttgcactgggaaaggtttatgtgcaatctggattgccaggaggagccggactcatgcatctcggagaagctgttcatggagatggcggaacttatggtatcggagggatggaaggatgccgggtatgagtatctctgtatcgacgattgttggatggctccccagagagactccgagggacgactccaagcggacccccagcgctttccacatggcattcgacagctcgccaattacgtgcactcgaaggggttgaagttgggaatctacgcagatgtgggcaacaaaacgtgtgcggggttcccggggtcgtttggatactacgatattgatgcgcagacgtttgctgactggggtgtcgatcttttgaaatttgatggctgttactgtgattcgttggaaaacctggcggatggatacaagcatatgtcactcgccttgaaccggacaggtcgctcaatcgtatacagctgcgaatggcccctctatatgtggcccttccaaaagcccaattacacagagattcggcagtattgcaatcactggaggaactttgccgatattgacgacagctggaaatccatcaagtccattctcgattggacgagcttcaaccaggagcgcatcgtggacgtggcaggacccggaggttggaacgatccggacatgctcgtaattgggaatttcgggcttagctggaatcagcaagtcacccaaatggcgctgtgggccatcatggcagctcctctctttatgtcgaatgatctgcggcatatctcgccccaggcaaaggctcttttgcaagacaaggacgtcatcgcaatcaatcaggacccattggggaaacagggatatcaacttcgccagggtgacaatttcgaagtatgggagaggccgcttagcgggctggcgtgggcggtcgcgatgattaaccggcaggaaatcggagggcctcgctcgtataccatcgcagtggcctcactgggcaaaggagtggcgtgcaatccggcctgcttcatcacccagttgttgcccgtcaaaagaaagctgggtttctacgagtggacatccagacttagatcacacattaaccctactggtacggtgttgctccagctcgaaaacacaatgcagatgtcgttgaaagacctgctgtaatctagaggatctcgagagatctaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaaatcatcgtcctttccttggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctgggatctctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggaccggtctcgagatccactagggccgcaggaacccctagtgatggagttggccactccctctctgcgcgctcgctcgctcactgaggccgcccgggctttgcccgggcggcctcagtgagcgagcgagcgcgcag

SEQ ID NO: 10 - protein, 31 aa, synthetic construct
MQLRNPELHLGCALALRFLALVSWDIPGARA

(19) United States
(12) Patent Application Publication
(10) Pub. No.: US 2025/0144240 A1
(43) Pub. Date: May 8, 2025

(54) METHODS FOR USE OF VIRAL VECTOR CONSTRUCTS FOR THE TREATMENT OF FABRY DISEASE

(71) Applicant: SANGAMO THERAPEUTICS, INC., Richmond, CA (US)

(72) Inventors: Cristobal PASSALACQUA, Richmond, CA (US); Marshall HUSTON, Richmond, CA (US); Bernard SOUBERBIELLE, Richmond, CA (US)

(21) Appl. No.: 18/707,085

(22) PCT Filed: Nov. 3, 2022
(86) PCT No.: PCT/US2022/079249

§ 371 Date: May 2, 2024

Related U.S. Application Data
(60) Provisional application No. 63/373,826, filed on Aug. 29, 2022.
(60) Provisional application No. 63/275,390, filed on Nov. 3, 2021.

(51) Int. Cl. A61K 48/00; A61K 31/573; A61K 38/47; A61P 3/00; C12N 15/86; G01N 33/543; G01N 33/573; G01N 33/68

(52) U.S. Cl. CPC A61K 48/005; A61K 31/573; A61K 38/47; A61K 48/0083; A61P 3/00; C12N 15/86; C12Y 302/01022; G01N 33/543; G01N 33/573; G01N 33/6854; C12N 2750/14143; C12N 2830/15; C12N 2830/48; C12N 2830/50; G01N 2333/924

(57) ABSTRACT

The present disclosure relates to the methods for treating or ameliorating one or more symptoms of Fabry disease, reducing the amount of glycosphingolipids, and/or increasing an α galactosidase A (α-Gal A) protein activity in a subject in need thereof by administering the expression vectors (e.g., an AAV expression vector) comprising an α galactosidase A (α-Gal A) expression cassette, which comprises an α galactosidase A (α-Gal A) transgene encoding the at least one α-Gal A protein at a dose of about 5×10¹² vector genomes per kilogram of body weight (vg/kg) to about 5×10¹³ vg/kg.

### CROSS REFERENCE TO RELATED APPLICATIONS

This application claims the benefit of the filing date of U.S. Provisional Patent Application No. 63/275,390, filed Nov. 3, 2021 and U.S. Provisional Patent Application No. 63/373,826, filed Aug. 29, 2022, the contents of which are incorporated herein by reference in their entirety.

### REFERENCE TO SEQUENCE LISTING SUBMITTED ELECTRONICALLY

The content of the electronically submitted sequence listing in ASCII text file (Name: 4341_024PC02_Seqlisting_ST26; Size: 16,280 bytes; and Date of Creation: Nov. 2, 2022) filed with the application is incorporated herein by reference in its entirety.

### FIELD OF DISCLOSURE

The present disclosure relates to the methods for treating or ameliorating one or more symptoms of Fabry disease, reducing the amount of glycosphingolipids, and/or increasing an α galactosidase A (α-Gal A) protein activity in a subject in need thereof by administering a therapeutically effective amount of the expression vectors (e.g., an adeno-associated virus (AAV) expression vector) comprising an α galactosidase A (α-Gal A) transgene encoding the at least one α-Gal A protein.

### BACKGROUND OF THE DISCLOSURE

Fabry disease is an X-linked lysosomal storage disorder resulting from a deficiency of the enzyme α-Galactosidase A (GLA). The resulting failure to hydrolyze the terminal α-galactosyl moiety from globotriaosylceramide (Gb3) causes accumulation of Gb3 in lysosomes and elsewhere in the cell. Early characteristic clinical manifestations include severe neuropathic pain (acroparesthesia), skin lesions (angiokeratomas), and ocular signs (cornea verticillata). Later in life, cardiac, renal, and cerebrovascular complications are responsible for severe morbidity and a shortened lifespan. Desnick et al., α-Galactosidase A Deficiency: Fabry Disease, in: Beaudet et al. (Ed.), The Online Metabolic and Molecular Bases of Inherited Disease, The McGraw-Hill Companies, Inc., New York, NY (2014); Van der Veen S J et al., Mol Genet Metab. 126(2): 162-168 (2019).

Since 2001, patients with Fabry disease have been treated with two different enzyme replacement therapies (ERTs), based on infusion of recombinant enzymes (agalsidase-α and agalsidase-β). Eng et al., N Engl J Med 345: 9-16 (2001); Schiffmann et al., JAMA 285:2743-2749 (2001); Lenders et al., J Am Soc Nephrol. 27(1): 256-64 (2016). Such treatment is only to treat the symptoms and is not curative, thus the patient must be given repeated dosing of these proteins for the rest of their lives.

Studies suggested that infusion of recombinant enzyme can lead to formation of the anti-GLA neutralizing antibodies, resulting in short-term acute complications, as well as deleterious long-term effects by therapy inhibition, resulting in severely decreased Gb3 and lyso-Gb3 depletion. Lenders et al., J Am Soc Nephrol. 27(1): 256-64 (2016). In male patients with classical Fabry disease, treatment with ERT delays the occurrence of complications, especially when treatment is initiated before the onset of irreversible organ damage. However, more than a half of classically affected male patients treated with ERT develop anti-GLA neutralizing antibodies. In female patients and patients with a non-classical disease phenotype, antibody formation against the administered recombinant enzyme is rarely observed. Van der Veen S J et al., Mol Genet Metab. 126(2): 162-168 (2019).

Accordingly, there remains a need for non-ERT methods and compositions that can be used to treat Fabry disease, including treatment through genome editing, for instance, to deliver an expressed transgene encoded gene product at a therapeutically relevant level.

### SUMMARY OF THE DISCLOSURE

In some aspects, provided herein is a method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg.

In some aspects, provided herein is a method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg.

In some aspects, provided herein is a method of reducing the amount of glycosphingolipids in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

In some aspects, provided herein is a method of reducing the amount of glycosphingolipids in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

In some aspects, provided herein is a method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α-Gal A transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

In some aspects, provided herein is a method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an α-Gal A transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

In some aspects, the subject has Fabry disease.

In some aspects, the α-Gal A expression cassette further comprises a mutated Woodchuck Hepatitis Virus (WHV) Posttranscriptional Regulatory Element (WPRE) sequence.

In some aspects, the mutated WPRE sequence comprises a mut6 mutated WPRE sequence.

In some aspects, the α-Gal A expression cassette further comprises an apolipoprotein E (APOE) enhancer operably linked to an alpha 1-antitrypsin (hAAT) promoter, a human hemoglobin beta (HBB)-IGG intron, a sequence encoding a signal peptide, and a bovine growth hormone poly A signal sequence.

In some aspects, the transgene comprises a wild-type α-Gal A sequence or a codon-optimized α-Gal A sequence.

In some aspects, the signal peptide is an α-GalA signal peptide.

In some aspects, the enhancer comprises the nucleotide sequence as set forth in SEQ ID NO: 2, the promotor comprises the nucleotide sequence as set forth in SEQ ID NO: 3, the intron comprises the nucleotide sequence as set forth in SEQ ID NO: 4, the α-GalA transgene comprises the nucleotide sequence as set forth in SEQ ID NO: 5, the mutated WPRE sequence comprises the nucleotide sequence as set forth in SEQ ID NO: 6, and the poly A signal sequence comprises the nucleotide sequence as set forth in SEQ ID NO: 7.

In some aspects, the enhancer comprises the nucleotide sequence as set forth in SEQ ID NO: 2, the promotor comprises the nucleotide sequence as set forth in SEQ ID NO: 3, the intron comprises the nucleotide sequence as set forth in SEQ ID NO: 4, the mutated WPRE sequence comprises the nucleotide sequence as set forth in SEQ ID NO: 6, and the poly A signal sequence comprises the nucleotide sequence as set forth in SEQ ID NO: 7.

In some aspects, the α-Gal A expression cassette comprises the nucleotide sequence as set forth in SEQ ID NO: 9.

In some aspects, the AAV expression vector serotype is AAV2/6.

In some aspects, the α-Gal A expression cassette is flanked on each end by inverted terminal repeats (ITRs). In some aspects, the ITRs are derived from adeno-associated virus type 2 (AAV2). In some aspects, the AAV expression vector further comprises the α-Gal A expression cassette flanked on each end by ITRs derived from AAV2, wherein the α-Gal A expression cassette is packaged with capsid derived from adeno-associated virus type 6 (AAV6).

In some aspects, the subject has one or more of the following symptoms: globotriaosylceramide (Gb3) levels above normal, globotriaosylsphingosine (lyso-Gb3) levels above normal, renal disease, cardiac disease, anhidrosis, acroparesthesia, angiokeratoma, gastrointestinal (GI) tract pain, corneal and lenticular opacities, or cerebrovascular disease. In some aspects, angiokeratoma is periumbilical angiokeratoma.

In some aspects, the subject has the α-GalA protein activity of less than about 5%.

In some aspects, the α-GalA protein activity is measured in the subject's plasma and/or leukocytes.

In some aspects, the subject is a male subject. In some aspects, the subject is a female subject.

In some aspects, the subject has an α-GalA gene mutation that is indicative of Fabry disease. In some aspects, the α-GalA gene mutation results in the amino acid mutation G261D, C422T, W340R, S297Y, Q283X, D215S, IVS5/c.801+3A>G, P362L, C422T, or N34S.

In some aspects, the subject has pre-existing anti-α-GalA antibodies prior to the administering as determined by an enzyme-linked immunosorbent assay (ELISA).

In some aspects, the subject is an anti-α-GalA neutralizing antibody positive subject when a biological sample of the subject is analyzed, wherein the anti-α-GalA neutralizing antibody positive subject has a biological sample having greater than about 9.6% inhibition of α-Galactosidase A activity as measured by an anti-α-GalA neutralizing antibody assay.

In some aspects, the α-Gal A protein expressed from the transgene decreases the amount of glycosphingolipids in the subject by at least about 2 fold as compared to the amount of glycosphingolipids in the subject prior to the administration.

In some aspects, wherein the α-Gal A protein expressed from the transgene decreases the amount of glycosphingolipids in the subject by about 10 percent (%), about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% as compared to the amount of glycosphingolipids in the subject prior to the administration.

In some aspects, the α-Gal A protein expressed from the transgene maintains the amount of glycosphingolipids in the subject at the same level as prior to the administration.

In some aspects, glycosphingolipids comprise globotriaosylceramide (Gb3), globotriaosylsphingosine (lyso-Gb3), galabiosylceramide, or any combination thereof.

In some aspects, Gb3 and/or lyso-Gb3 levels are measured in the subject's plasma and/or urine.

In some aspects, Gb3 and/or lyso-Gb3 levels are measured in the subject's tissue.

In some aspects, the α-Gal A protein expressed from the transgene decreases the amount of glycosphingolipids in one or more of plasma, liver, heart, kidney, urine, skin, or spleen.

In some aspects, the α-Gal A protein activity in the subject is between about 0-fold higher to about 2-fold higher, between about 2-fold higher to about 5-fold higher, between about 5-fold higher to about 10-fold higher, between about 10-fold higher to about 20-fold higher, between about 20-fold higher to about 30-fold higher, between about 30-fold higher to about 40-fold higher, between about 30-fold higher to about 40-fold higher, between about 40-fold higher to about 50-fold higher, between about 50-fold higher to about 60-fold higher, between about 60-fold higher to about 70-fold higher, between about 70-fold higher to about 80-fold higher, between about 80-fold higher to about 90-fold higher, between about 90-fold higher to about 100-fold higher, between about 100-fold higher to about 200-fold higher, between about 200-fold higher to about 300-fold higher, between about 300-fold higher to about 400-fold higher, between about 400-fold higher to about 500-fold higher than the mean normal α-Gal A protein activity compared to the α-Gal A protein activity in the subject prior to the administration.

In some aspects, the α-Gal A protein activity in the subject is between about 0-fold higher to about 2-fold higher, between about 2-fold higher, about 3-fold higher, about 4-fold higher, about 5-fold higher, about 6-fold higher, about 7-fold higher, about 8-fold higher, about 9-fold higher, about 10-fold higher, about 11-fold higher, about 12-fold higher, about 13-fold higher, about 14-fold higher, about 15-fold higher, about 16-fold higher, about 17-fold higher, about 18-fold higher, about 19-fold higher, about 20-fold higher, about 21-fold higher, about 22-fold higher, about 23-fold higher, about 24-fold higher, about 25-fold higher, about 26-fold higher, about 27-fold higher, about 28-fold higher, about 29-fold higher, about 30-fold higher, about 31-fold higher, about 32-fold higher, about 33-fold higher, about 34-fold higher, about 35-fold higher, about 36-fold higher, about 37-fold higher, about 38-fold higher, about 39-fold higher, about 40-fold higher, about 41-fold higher, about 42-fold higher, about 43-fold higher, about 44-fold higher, about 45-fold higher, about 46-fold higher, about 47-fold higher, about 48-fold higher, about 49-fold higher, about 50-fold higher, about 51-fold higher, about 52-fold higher, about 53-fold higher, about 54-fold higher, about 55-fold higher, about 56-fold higher, about 57-fold higher, about 58-fold higher, about 59-fold higher, about 60-fold higher, about 65-fold higher, about 66-fold higher, about 67-fold higher, about 68-fold higher, about 69-fold higher, about 70-fold higher, about 71-fold higher, about 72-fold higher, about 73-fold higher, about 74-fold higher, about 75-fold higher, about 76-fold higher, about 77-fold higher, about 78-fold higher, about 79-fold higher, about 80-fold higher, about 81-fold higher, about 82-fold higher, about 83-fold higher, about 84-fold higher, about 85-fold higher, about 86-fold higher, about 87-fold higher, about 88-fold higher, about 89-fold higher, about 90-fold higher, about 91-fold higher, about 92-fold higher, about 93-fold higher, about 94-fold higher, about 95-fold higher, about 96-fold higher, about 97-fold higher, about 98-fold higher, about 99-fold higher, or about 100-fold higher than the mean normal α-Gal A protein activity compared to the α-Gal A protein activity in the subject prior to the administration.

In some aspects, the levels of the α-Gal A protein expressed from the transgene are measured in one or more of the subject's plasma, serum, whole blood, dried blood spot, leukocytes, or other blood components.

In some aspects, the α-Gal A protein expressed from the transgene is active in the subject's kidneys, liver, skin, and heart.

In some aspects, the AAV expression vector is administered parenterally. In some aspects, the AAV expression vector is administered intravenously.

In some aspects, the AAV expression vector is administered in a pharmaceutically acceptable carrier. In some aspects, the pharmaceutically acceptable carrier comprises phosphate buffered saline containing CaCl₂, MgCl₂, NaCl, Sucrose, and Kolliphor (Poloxamer) P 188.

In some aspects, only one dose of the AAV expression vector is administered to the subject. In some aspects, the AAV expression vector is administered at a dose of about 5×10¹² vg/kg. In some aspects, the AAV expression vector is administered at a dose of about 1×10¹³ vg/kg. In some aspects, the AAV expression vector is administered at a dose of about 3×10¹³ vg/kg. In some aspects, the AAV expression vector is administered at a dose of about 5×10¹³ vg/kg.

In some aspects, the subject is administered an immunosuppressant prior to and/or during administration of the AAV expression vector. In some aspects, the immunosuppressant comprises prednisone.

In some aspects, the subject is not administered an immunosuppressant prior to and/or during administration of the AAV expression vector.

In some aspects, the subject is not administered a preconditioning treatment prior to the administration of the AAV expression vector.

In some aspects, the expression of the at least one α-Gal A protein is sustained for at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

In some aspects, an Estimated Glomerular filtration rate (eGFR) in ml/min/1.73 m² is measured in the subject after the administering by using the Chronic Kidney Disease Epidemiology Collaboration (CKD-EPI) equation. In some aspects, the rate of annual eGFR decline is lower than in a comparable untreated subject with Fabry disease.

In some aspects, an Ejection Fraction (EF) is measured in the subject as stroke volume (SV)/left ventricular volumes at end-diastole (LVEDV) after the administering. In some aspects, the rate of annual EF decline is lower than in a comparable untreated subject with Fabry disease.

In some aspects, a Global Longitudinal Strain (GLS) is measured in the subject by a two-dimensional (2D) strain echocardiography or cardiac magnetic resonance imaging (cardiac MRI or CMR) after the administration. In some aspects, the annual shortening progression the contractibility of the muscles of the heart is lower than in a comparable untreated subject with Fabry disease.

In some aspects, a relaxation times of the myocardium is measured in the subject by native T1 mapping on cardiac magnetic resonance imaging (cardiac MRI or CMR) after the administration. In some aspects, the annual decrease in the relaxation time is lower than in a comparable untreated subject with Fabry disease.

In some aspects, edema as the increased water content in the myocardium is measured in the subject by T2 mapping on cardiac magnetic resonance imaging (cardiac MRI or CMR) after the administration. In some aspects, the annual increase in the water content is lower than in a comparable untreated subject with Fabry disease.

In some aspects, a Left Ventricular Mass Index (LVMI) is measured as left ventricular mass (LVM)/body surface area in the subject after the administering. In some aspects, the LVMI is the annual LVMI increase is lower than in a comparable untreated subject with Fabry disease.

In some aspects, there is an improvement in one or more audiologic symptoms in the subject after the administration. In some aspects, one or more audiologic symptoms are tinnitus, vertigo, or progressive hearing loss.

In some aspects, the subject had a positive change in the level of perspiration from anhidrosis to hypohidrosis or normal hidrosis.

In some aspects, the subject has been administered with an enzyme replacement therapy (ERT) for Fabry disease prior to the administering (“pre-treatment”). In some aspects, the enzyme replacement therapy comprises a recombinant α-Galactosidase A (GLA) protein or a gene expressing GAL. In some aspects, the enzyme replacement therapy for the pre-treatment comprises administering galafold, AVR-RD-01, FLT-190, pegunigalsidase alfa, 4D-310, or any combination thereof.

In some aspects, the enzyme replacement therapy for the pre-treatment comprises a recombinant α-Galactosidase A (GLA) protein in combination with an active site-specific chaperone (ASSC) for the GLA. In some aspects, the ASSC is 1-deoxygalactonojirimycin.

In some aspects, the enzyme replacement therapy for the pre-treatment comprises agalsidase alpha and/or beta or a gene expressing agalsidase alpha and/or beta. In some aspects, the enzyme replacement therapy for the pre-treatment comprises fabrazyme, Replagal, PRX-102, or any combination thereof.

In some aspects, the enzyme replacement therapy for the pre-treatment comprises a gene therapy. In some aspects, the gene therapy comprises a vector encoding the enzyme. In some aspects, the gene therapy comprises administering AVR-RD-01, FLT-190, pegunigalsidase alfa, 4D-310, or any combination thereof.

In some aspects, the vector comprises an mRNA encoding a human GLA protein or agalsidase alpha and/or beta. In some aspects, the vector is a viral vector. In some aspects, the viral vector comprises an adeno-associated virus (AAV) vector or a lentiviral vector. In some aspects, the gene therapy is delivered by a lipid nanoparticle.

In some aspects, the subject has been administered with a non-enzyme replacement therapy for Fabry disease prior to the administering (“pre-treatment”). In some aspects, the for the pre-treatment therapy for Fabry disease comprises lucerastat, venglustat, apabetalone, or any combination thereof.

In some aspects, Fabry disease is type 1 classic phenotype or type 2 later-onset phenotype.

### BRIEF DESCRIPTION OF THE DRAWINGS

FIG. 1 shows a schematic of the AAV-001 human α galactosidase A (hGLA) AAV cassette comprising liver-specific regulatory elements (e.g., an enhancer, a promoter, an intron), an α galactosidase A (α-Gal A) transgene encoding the at least one α-Gal A protein (human alpha galactosidase A), a mutated form of the woodchuck hepatitis virus (WHV) posttranscriptional regulatory element (WPREmut6), a polyadenylation (polyA) signal sequence, which is flanked by ITR sequences. SP refers to the endogenous hGLA signal peptide. Sizes of the various elements are shown as is the entire size of the cassette (3321 bp).

FIG. 2 is a schematic of a phase 1/2, global, open-label, single-dose, dose-ranging multicenter study to assess the safety and tolerability of AAV-001 (an AAV2/6 human α-Gal A gene therapy) in patients with Fabry disease as described in Example 1.

FIG. 3 shows baseline patient characteristics (age (years), ERT status, plasma α-Gal A protein activity (nmol/h/ml), plasma lyso-Gb3 (ng/ml), primary disease signs and symptoms, renal function (eGFR), pre-existing α-Gal A antibodies, and α-Gal A amino acid mutations) for patients 1-9 cohorts 1-4 and patient 10 in expansion cohort. Ab=antibody; CKD-EPI=Chronic Kidney Disease Epidemiology Collaboration, eGFR=estimated glomerular filtration rate; LOD=limit of detection. ^{†}Baseline values were considered the timepoint immediately preceding AAV-001 administration. teGFR measured as CKD-EPI (mL/min/1.73m2).

FIG. 4A shows safety and tolerability data related to AAV-001 treatment. All safety data were evaluated from the four patients in the first 2 dose cohorts (0.5e13 vg/kg and 1e13 vg/kg) as of the cutoff date (e.g., date of the last measured point).

FIG. 4B shows safety and tolerability data related to AAV-001 treatment. All safety data were evaluated from the five patients in dose cohorts 1-3 (0.5e13 vg/kg, 1.0e13 vg/kg, and 3.0e13 vg/kg) as of the cutoff date. Length of follow up ranged from 4-52 weeks (subjects 1 and 2, 52 weeks; subject 3, 40 weeks; subject 4, 25 weeks; subject 5, 3 weeks). MedDRA=Medical Dictionary for Regulatory Activities; vg/kg=vector genomes per kilogram of body weight.

FIG. 4C shows safety and tolerability data related to AAV-001 treatment. All safety data were evaluated from the nine patients in dose cohorts 1-4 (0.5e13 vg/kg, 1.0e13 vg/kg, 3.0e13 vg/kg and 5.0e13 vg/kg, respectively) as of the cutoff date. Length of follow up ranged from 4 weeks to 15 months. MedDRA=Medical Dictionary for Regulatory Activities; vg/kg=vector genomes per kilogram of body weight.

FIG. 5A shows plasma α-Gal A protein activity (nmol/h/ml) measured in patients 1-4 as described in Example 2. ERT=enzyme replacement therapy; vg/kg=vector genomes per kilogram of body weight.

FIG. 5B shows plasma α-Gal A protein activity (nmol/h/ml) measured in patients 1-5 as described in Example 2. ERT=enzyme replacement therapy; vg/kg=vector genomes per kilogram of body weight.

FIG. 5C shows plasma α-Gal A protein activity (nmol/h/ml) measured in patients 1-8 as described in Example 2. ERT=enzyme replacement therapy; vg/kg-vector genomes per kilogram of body weight.

FIG. 6A shows plasma α-Gal A protein activity (nmol/h/ml) and Lyso-Gb3 concentration (ng/ml) measured at week 12 and 48 after AAV-001 administration in patient 1, as described in Example 2.

FIG. 6B shows plasma α-Gal A protein activity (nmol/h/ml) and Lyso-Gb3 concentration (ng/ml) measured at week 52 after AAV-001 administration in patient 1, as described in Example 2.

FIG. 6C shows plasma α-Gal A protein activity (nmol/h/ml) and Lyso-Gb3 concentration (ng/ml) measured over time after AAV-001 administration in patient 1, as described in Example 2.

FIG. 6D shows plasma α-Gal A protein activity (nmol/h/ml) and Lyso-Gb3 concentration (ng/ml) measured at week 12 and 48 after AAV-001 administration in patient 2, as described in Example 2.

FIG. 6E shows plasma α-Gal A protein activity (nmol/h/ml) and Lyso-Gb3 concentration (ng/ml) measured at week 52 after AAV-001 administration in patient 2, as described in Example 2.

FIG. 6F shows plasma α-Gal A protein activity (nmol/h/ml) and Lyso-Gb3 concentration (ng/ml) measured over time after AAV-001 administration in patient 2, as described in Example 2.

FIG. 6G shows plasma α-Gal A protein activity (nmol/h/ml) and Lyso-Gb3 concentration (ng/ml) measured at week 12 and 28 after AAV-001 administration in patient 3, as described in Example 2.

FIG. 6H shows plasma α-Gal A protein activity (nmol/h/ml) measured at week 40 and Lyso-Gb3 concentration (ng/ml) measured at week 36 after AAV-001 administration in patient 3, as described in Example 2.

FIG. 6I shows plasma α-Gal A protein activity (nmol/h/ml) and Lyso-Gb3 concentration (ng/ml) measured over time after AAV-001 administration in patient 3, as described in Example 2.

FIG. 6J shows plasma α-Gal A protein activity (nmol/h/ml) and Lyso-Gb3 concentration (ng/ml) measured at week 12 after AAV-001 administration in patient 4, as described in Example 2.

FIG. 6K shows plasma α-Gal A protein activity (nmol/h/ml) and Lyso-Gb3 concentration (ng/ml) measured over time after AAV-001 administration in patient 4, as described in Example 2.

FIG. 6L shows plasma α-Gal A protein activity (nmol/h/ml) measured at week 25 and Lyso-Gb3 concentration (ng/ml) measured at week 20 after AAV-001 administration in patient 4, as described in Example 2.

FIG. 6M shows plasma α-Gal A protein activity (nmol/h/ml) and Lyso-Gb3 concentration (ng/ml) measured over time after AAV-001 administration in patient 5, as described in Example 2.

FIG. 6N shows plasma α-Gal A protein activity (nmol/h/ml) and Lyso-Gb3 concentration (ng/ml) measured over time after AAV-001 administration in patient 6, as described in Example 2.

FIG. 6O shows plasma α-Gal A protein activity (nmol/h/ml) and Lyso-Gb3 concentration (ng/ml) measured over time after AAV-001 administration in patient 7, as described in Example 2.

FIG. 6P shows plasma α-Gal A protein activity (nmol/h/ml) and Lyso-Gb3 concentration (ng/ml) measured over time after AAV-001 administration in patient 8, as described in Example 2.

FIG. 6Q shows Lyso-Gb3 concentration (ng/ml) measured over time after AAV-001 administration in patient 9, as described in Example 2.

### DETAILED DESCRIPTION OF THE DISCLOSURE

The present disclosure relates to the methods for treating or ameliorating one or more symptoms of Fabry disease, reducing the amount of glycosphingolipids, and/or increasing an α galactosidase A (α-Gal A) protein activity in a subject in need thereof by administering the expression vectors (e.g., an AAV expression vector) comprising an α galactosidase A (α-Gal A) expression cassette, which comprises an α galactosidase A (α-Gal A) transgene encoding the at least one α-Gal A protein at a dose of about 5×10¹² vector genomes per kilogram of body weight (vg/kg) to about 5×10¹³ vg/kg.

### I. Definitions

In order that the present disclosure can be more readily understood, some terms are first defined. As used in this application, except as otherwise expressly provided herein, each of the following terms shall have the meaning set forth below. Additional definitions are set forth throughout the application.

It is to be noted that the term “a” or “an” entity refers to one or more of that entity; for example, “a nucleic acid sequence,” is understood to represent one or more nucleic acid sequences, unless stated otherwise. As such, the terms “a” (or “an”), “one or more,” and “at least one” can be used interchangeably herein.

Furthermore, “and/or”, where used herein, is to be taken as specific disclosure of each of the two specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

It is understood that wherever aspects are described herein with the language “comprising,” otherwise analogous aspects described in terms of “consisting of” and/or “consisting essentially of” are also provided.

As used herein, “about” will be understood by persons of ordinary skill and will vary to some extent depending on the context in which it is used. If there are uses of the term which are not clear to persons of ordinary skill given the context in which it is used, “about” will mean up to plus or minus 10% of the particular value.

The term “at least” prior to a number or series of numbers is understood to include the number adjacent to the term “at least,” and all subsequent numbers or integers that could logically be included, as clear from context. For example, the number of nucleotides in a nucleic acid molecule must be an integer. For example, “at least 18 nucleotides of a 21-nucleotide nucleic acid molecule” means that 18, 19, 20, or 21 nucleotides have the indicated property. When at least is present before a series of numbers or a range, it is understood that “at least” can modify each of the numbers in the series or range. “At least” is also not limited to integers (e.g., “at least 5%” includes 5.0%, 5.1%, 5.18% without consideration of the number of significant figures).

As used herein, “no more than” or “less than” is understood as the value adjacent to the phrase and logical lower values or integers, as logical from context, to zero. When “no more than” is present before a series of numbers or a range, it is understood that “no more than” can modify each of the numbers in the series or range.

As used herein, the term “immune response” refers to a biological response within an organism against a foreign agent or abnormal cell (e.g., Fabry disease cell), wherein the response protects the organism against such agents/cells and diseases caused by them. An immune response is mediated by the action of a cell of the immune system (e.g., a T lymphocyte (T cell), B lymphocyte (B cell), natural killer (NK) cell, macrophage, eosinophil, mast cell, dendritic cell or neutrophil) and soluble macromolecules produced by any of these cells or the liver (including antibodies, cytokines, and complement) that results in selective targeting, binding to, damage to, destruction of, and/or elimination from the organism's body of invading pathogens, cells or tissues infected with pathogens, Fabry disease cells, or other abnormal cells, or, in cases of autoimmunity or pathological inflammation, normal human cells or tissues. In some aspects, an immune reaction includes, e.g., activation or inhibition of a T cell, e.g., an effector T cell or a Th cell, such as a CD4⁺ or CD8⁺ T cell, or the inhibition of a regulatory T cell (Treg cell).

The term “vector” or “delivery vector” as used herein, is intended to refer to a nucleic acid molecule capable of transporting another nucleic acid to which it has been linked. The term “vector,” or “delivery vector” includes both viral and nonviral vehicles for introducing the nucleic acid into a cell in vitro, ex vivo or in vivo. A large number of vectors are known and used in the art including, for example, plasmids, modified eukaryotic viruses, or modified bacterial viruses. One type of vector is a “plasmid,” which refers to a circular double stranded DNA loop into which additional DNA segments can be ligated. Another type of vector is a viral vector, wherein additional DNA segments can be ligated into the viral genome. Some vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, some vectors are capable of directing the expression of genes to which they are operatively linked. Such vectors are referred to herein as “recombinant expression vectors” (or simply, “expression vectors,” otherwise known as “expression constructs”). In general, expression vectors of utility in recombinant DNA techniques are often in the form of plasmids. In the present disclosure, “plasmid” and “vector” can be used interchangeably as the plasmid is the most commonly used form of vector. However, also included are other forms of expression vectors, such as viral vectors (e.g., replication defective retroviruses, adenoviruses, adeno-associated viruses (“AAVs”), and lentiviruses), which serve equivalent functions.

The term “expression vector” or “expression construct” or means any type of genetic construct containing a nucleic acid in which part or all of the nucleic acid encoding sequence is capable of being transcribed.

A “viral vector” refers to a sequence that comprises one or more polynucleotide regions encoding or comprising a molecule of interest, e.g., a protein, a peptide, and an oligonucleotide or a plurality thereof. Viral vectors can be used to deliver genetic materials into cells. Viral vectors can be modified for specific applications. In some aspects, the delivery vector of the disclosure is a viral vector selected from the group consisting of an adeno-associated virus (AAV) vector, an adenoviral vector, a lentiviral vector, or a retroviral vector.

The term “adeno-associated virus vector” or “AAV vector” as used herein refers to any vector that comprises or derives from components of an adeno-associated virus and is suitable to infect mammalian cells, preferably human cells. The term AAV vector typically designates an AAV-type viral particle or virion comprising a payload. The AAV vector can be derived from various serotypes, including combinations of serotypes (i.e., “pseudotyped” AAV) or from various genomes (e.g., single stranded or self-complementary).

The term “AAV2/6,” “rAAV2/6,” or “pseudotyped AAV2/6” vector as used herein refers to the AAV expression vector of the present disclosure (e.g., AAV-001 rAAV vector) comprising the α-Gal A expression cassette flanked on each end by ITRs derived from AAV2, wherein the α-Gal A expression cassette is packaged with capsid derived from adeno-associated virus type 6 (AAV6).

In addition, the AAV vector can be replication defective and/or targeted. As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAVrh8, AAVrh10, AAVrh.74, snake AAV, avian AAV, bovine AAV, canine AAV, equine AAV, ovine AAV, goat AAV, shrimp AAV, those AAV serotypes and clades disclosed by Gao et al. (J. Virol. 78:6381 (2004)) and Moris et al. (Virol. 33:375 (2004)), and any other AAV now known or later discovered. See, e.g., FIELDS et al. VIROLOGY, volume 2, chapter 69 (4th ed., Lippincott-Raven Publishers). In some aspects, an “AAV vector” includes a derivative of a known AAV vector. In some aspects, an “AAV vector” includes a modified or an artificial AAV vector. In some aspects, an “AAV vector” includes a recombinant adeno-associated virus (rAAV). In some aspects, the AAV expression vector serotype is AAV2/6.

In some aspects, the AAV vector is modified or mutated relative to the wild-type AAV serotype sequence.

Insertion of a polynucleotide into a suitable vector can be accomplished by ligating the appropriate polynucleotide fragments into a chosen vector that has complementary cohesive termini. Vectors can be engineered to encode selectable markers or reporters that provide for the selection or identification of cells that have incorporated the vector. Expression of selectable markers or reporters allows identification and/or selection of host cells that incorporate and express other coding regions contained on the vector. Examples of selectable marker genes known and used in the art include: genes providing resistance to ampicillin, streptomycin, gentamycin, kanamycin, hygromycin, bialaphos herbicide, sulfonamide, and the like; and genes that are used as phenotypic markers, i.e., anthocyanin regulatory genes, isopentanyl transferase gene, and the like. Examples of reporters known and used in the art include: luciferase (Luc), green fluorescent protein (GFP), chloramphenicol acetyltransferase (CAT), β-galactosidase (LacZ), β-glucuronidase (Gus), and the like. Selectable markers can also be considered to be reporters. In some aspects, the delivery vector is selected from the group consisting of a viral vector (e.g., an AAV vector), a plasmid, a lipid, a protein particle, a bacterial vector, a lysosome, a virus-like particle, a polymeric particle, an exosome, or a vault particle.

Some aspects of the disclosure are directed to biological vectors, which can include viruses, particularly attenuated and/or replication-deficient viruses.

As used herein, the term “promoter” refers to a DNA sequence recognized by the machinery of the cell, or introduced synthetic machinery, required to initiate the transcription of a gene. The term “promoter” is also meant to encompass those nucleic acid elements sufficient for promoter-dependent gene expression controllable for cell-type specific, tissue-specific or inducible by external signals or agents; such elements can be located in the 5′ or 3′ regions of the native gene. In some aspects, the promoter is a constitutively active promoter, a cell-type specific promoter, or an inducible promoter. In some aspects, the promoter is an alpha 1-antitrypsin (hAAT) promoter.

In some aspects, microRNA targeting sequences are included to increase specificity of vector-mediated transgene expression. See e.g., Anja Geisler and Henry Fechner, World J Exp Med., 20; 6(2): 37-54 (2016).

As used herein, the term “enhancer” is a cis-acting element that stimulates or inhibits transcription of adjacent genes. An enhancer that inhibits transcription is also referred to as a “silencer.” Enhancers can function (e.g., can be associated with a coding sequence) in either orientation, over distances of up to several kilobase pairs (kb) from the coding sequence and from a position downstream of a transcribed region. In some aspects, the enhancer is an apolipoprotein E (APOE) enhancer. In some aspects, the APOE enhancer is operably linked to the hAAT promoter.

As used herein, the term “regulatable promoter” is any promoter whose activity is affected by a cis or trans acting factor (e.g., an inducible promoter, such as an external signal or agent).

As used herein, the term “constitutive promoter” is any promoter that directs RNA production in many or all tissue/cell types at most times, e.g., the human CMV immediate early enhancer/promoter region that promotes constitutive expression of cloned DNA inserts in mammalian cells.

The terms “transcriptional regulatory protein,” “transcriptional regulatory factor,” and “transcription factor” are used interchangeably herein, and refer to a nuclear protein that binds a DNA response element and thereby transcriptionally regulates the expression of an associated gene or genes. Transcriptional regulatory proteins generally bind directly to a DNA response element, however in some cases binding to DNA can be indirect by way of binding to another protein that in turn binds to, or is bound to a DNA response element.

As used herein, the term “termination signal sequence” can be any genetic element that causes RNA polymerase to terminate transcription, such as for example a polyadenylation (polyA or pA) signal sequence. A polyadenylation signal sequence is a recognition region necessary for endonuclease cleavage of an RNA transcript that is followed by the polyadenylation consensus sequence AATAAA. A polyadenylation signal sequence provides a “polyA site,” i.e., a site on a RNA transcript to which adenine residues will be added by post-transcriptional polyadenylation.

The terms “operatively linked,” “operatively inserted,” “operatively positioned,” “under control” or “under transcriptional control” means that the promoter is in the correct location and orientation in relation to the nucleic acid to control RNA polymerase initiation and expression of the gene. In some aspects, the term “operably linked” means that a DNA sequence and a regulatory sequence(s) are connected in such a way as to permit gene expression when the appropriate molecules (e.g., transcriptional activator proteins) are bound to the regulatory sequence(s). In some aspects, the term “operably inserted” means that the DNA of interest introduced into the cell is positioned adjacent a DNA sequence which directs transcription and translation of the introduced DNA (i.e., facilitates the production of, e.g., a polypeptide encoded by a DNA of interest).

A “coding sequence” or a sequence “encoding” a particular molecule (e.g., at least one α-Gal A protein) is a nucleic acid that is transcribed (in the case of DNA) or translated (in the case of mRNA) into polypeptide, in vitro or in vivo, when operably linked to an appropriate regulatory sequence, such as a promoter. The boundaries of the coding sequence are determined by a start codon at the 5′ (amino) terminus and a translation stop codon at the 3′ (carboxy) terminus. A coding sequence can include, but is not limited to, cDNA from prokaryotic or eukaryotic mRNA, genomic DNA sequences from prokaryotic or eukaryotic DNA, and synthetic DNA sequences. A transcription termination sequence will usually be located 3′ to the coding sequence.

The term “derived from,” as used herein, refers to a component that is isolated from or made using a specified molecule or organism, or information (e.g., amino acid or nucleic acid sequence) from the specified molecule or organism. For example, a nucleic acid sequence (e.g., an AAV vector) that is derived from a second nucleic acid sequence (e.g., another AAV vector) can include a nucleotide sequence that is identical or substantially similar to the nucleotide sequence of the second nucleic acid sequence.

In the case of a polynucleotide disclosed herein, the derived species can be obtained by, for example, naturally occurring mutagenesis, artificial directed mutagenesis or artificial random mutagenesis. The mutagenesis used to derive polynucleotides can be intentionally directed or intentionally random, or a mixture of each. The mutagenesis of a polynucleotide to create a different polynucleotide derived from the first can be a random event (e.g., caused by polymerase infidelity) and the identification of the derived polynucleotide can be made by appropriate screening methods.

As used herein, the term “mutation” refers to any changing of the structure of a gene, resulting in a variant (also called “mutant”) form that can be transmitted to subsequent generations. Mutations in a gene can be caused by the alternation of single base in DNA, or the deletion, insertion, or rearrangement of larger sections of genes or chromosomes.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties {e.g., phosphorothioate backbones). In general, an analogue of a particular nucleotide has the same base-pairing specificity; i.e., an analogue of A will base-pair with T. In some aspects, a nucleic acid molecule can be complementary DNA (cDNA). Polynucleotides can be made recombinantly, enzymatically, or synthetically, e.g., by solid-phase chemical synthesis followed by purification. When referring to a sequence of the polynucleotide or nucleic acid, reference is made to the sequence or order of nucleobase moieties, or modifications thereof, of the covalently linked nucleotides or nucleosides.

The term “cDNA,” as used herein, is a DNA copy of a messenger RNA (mRNA) molecule produced by reverse transcriptase, a DNA polymerase that can use either DNA or RNA as a template.

The term “mRNA,” as used herein, refers to a single stranded RNA that encodes the amino acid sequence of one or more polypeptide chains.

The nucleic acids can be present in whole cells, in a cell lysate, or in a partially purified or substantially pure form. A nucleic acid is “isolated” or “rendered substantially pure” when purified away from other cellular components or other contaminants, e.g., other cellular nucleic acids (e.g., the other parts of the chromosome) or proteins, by standard techniques, including alkaline/SDS treatment, CsCl banding, column chromatography, agarose gel electrophoresis and others well known in the art. See, F. Ausubel, et al., ed. Current Protocols in Molecular Biology, Greene Publishing and Wiley Interscience, New York (1987).

Nucleic acids, e.g., cDNA, can be mutated, in accordance with standard techniques to provide gene sequences. For coding sequences, these mutations, can affect amino acid sequence as desired. In particular, DNA sequences substantially homologous to or derived from native V, D, J, constant, switches and other such sequences described herein are contemplated (where “derived” indicates that a sequence is identical or modified from another sequence).

The term “antisense,” as used herein, refers to a nucleic acid that is sufficiently complementary to all or a portion of a gene, primary transcript, or processed mRNA, so as to interfere with expression of the endogenous gene. “Complementary” polynucleotides are those that are capable of base pairing according to the standard Watson-Crick complementarity rules. Specifically, purines will base pair with pyrimidines to form a combination of guanine paired with cytosine (G: C) and adenine paired with either thymine (A: T) in the case of DNA, or adenine paired with uracil (A: U) in the case of RNA. It is understood that two polynucleotides can hybridize to each other even if they are not completely complementary to each other, provided that each has at least one region that is substantially complementary to the other.

The terms “antisense strand” and “guide strand” refer to the strand of a dsRNA, e.g., an shRNA, that includes a region that is substantially complementary to a target sequence, e.g., mRNA. The antisense strand has sequence sufficiently complementary to the desired target mRNA sequence to direct target-specific silencing, e.g., complementarity sufficient to trigger the destruction of the desired target mRNA by the RNAi machinery or process.

The terms “sense strand” and “passenger strand,” as used herein, refer to the strand of a dsRNA, e.g., an shRNA, that includes a region that is substantially complementary to a region of the antisense strand as that term is defined herein. The antisense and sense strands of a dsRNA, e.g., an shRNA, are hybridized to form a duplex structure.

The term “gene,” as used herein, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

The term “transgene,” as used herein, refers to a segment of DNA from one organism introduced into the genome of another organism. Transgenes can be delivered to a cell by a variety of ways, such that the transgene becomes integrated into the cell's own genome and is maintained there. In recent years, a strategy for transgene integration has been developed that uses cleavage with site-specific nucleases for targeted insertion into a chosen genomic locus (see, e.g., U.S. Pat. No. 7,888,121). Nucleases, such as zinc finger nucleases (ZFNs), transcription activator-like effector nucleases (TALENs), or nuclease systems such as the RNA guided CRISPR/Cas system (utilizing an engineered guide RNA), are specific for targeted genes and can be utilized such that the transgene construct is inserted by either homology directed repair (HDR) or by end capture during non-homologous end joining (NHEJ) driven processes. See, e.g., U.S. Pat. Nos. 9,394,545; 9,255,250; 9,200,266; 9,045,763; 9,005,973; 9,150,847; 8,956,828; 8,945,868; 8,703,489; 8,586,526; 6,534,261; 6,599,692; 6,503,717; 6,689,558; 7,067,317; 7,262,054; 7,888,121; 7,972,854; 7,914,796; 7,951,925; 8,110,379; 8,409,861; U.S. Patent Publications 20030232410; 20050208489; 20050026157; 20050064474; 20060063231; 20080159996; 201000218264; 20120017290; 20110265198; 20130137104; 20130122591; 20130177983; 20130196373; 20140120622; 20150056705; 20150335708; 20160030477 and 20160024474, the disclosures of which are incorporated by reference in their entireties.

Transgenes can be introduced and maintained in cells in a variety of ways. Following a “cDNA” approach, a transgene is introduced into a cell such that the transgene is maintained extra-chromosomally rather than via integration into the chromatin of the cell. The transgene may be maintained on a circular vector (e.g. a plasmid, or a non-integrating viral vector such as AAV or lentivirus), where the vector can include transcriptional regulatory sequences such as promoters, enhancers, poly A signal sequences, introns, and splicing signals (U.S. Publication No. 20170119906). In some aspects, the AAV expression vectors of the present disclosure comprise an α galactosidase A (α-Gal A) transgene encoding the at least one α-Gal A protein. In some aspects, the transgene of the present disclosure comprises a wild-type α-Gal A sequence or a codon-optimized α-Gal A sequence. In some aspects, the α-GalA transgene of the present disclosure comprises the nucleotide sequence as set forth in SEQ ID NO: 5.

The term “gene expression,” as used herein, refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

The term “GLA gene,” as used herein, encodes at least one α-Galactosidase A (α-Gal A) protein, as discussed herein.

The term “modulation” of gene expression, as used herein, refers to a change in the activity of a gene. Modulation of the expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include e.g., a ZFP, TALE, or CRISPR/Cas system. Thus, gene inactivation can be partial or complete.

The term “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

The term “eukaryotic cell,” as used herein, includes but is not limited to a fungal cell (such as a yeast cell), a plant cell, an animal cell, a mammalian cell, and a human cell (e.g., a liver cell, muscle cell, red blood cell, etc.), including stem cells (pluripotent and multipotent).

The term “secretory tissues,” as used herein, are those tissues in an animal that secrete products out of the individual cell into a lumen of some type which are typically derived from epithelium. Examples of secretory tissues that are localized to the gastrointestinal tract include the cells that line the gut, the pancreas, and the gallbladder. Other secretory tissues include the liver, tissues associated with the eye and mucous membranes such as salivary glands, mammary glands, the prostate gland, the pituitary gland and other members of the endocrine system. Additionally, secretory tissues include individual cells of a tissue type, which are capable of secretion.

As used herein, the term “polypeptide” is intended to encompass a singular “polypeptide” as well as plural “polypeptides,” and comprises any chain or chains of two or more amino acids. Thus, as used herein, a “peptide,” a “peptide subunit,” a “protein,” an “amino acid chain,” an “amino acid sequence,” or any other term used to refer to a chain or chains of two or more amino acids, are included in the definition of a “polypeptide,” even though each of these terms can have a more specific meaning. The term “polypeptide” can be used instead of, or interchangeably with any of these terms. The term further includes polypeptides which have undergone post-translational or post-synthesis modifications, for example, conjugation of a palmitoyl group, glycosylation, acetylation, phosphorylation, amidation, derivatization by known protecting/blocking groups, proteolytic cleavage, or modification by non-naturally occurring amino acids. The term “peptide,” as used herein encompasses full length peptides and fragments, variants or derivatives thereof. A “peptide” as disclosed herein, can be part of a fusion polypeptide comprising additional components such as, e.g., an Fc domain or an albumin domain, to increase half-life. A peptide as described herein can also be derivatized in a number of different ways. A peptide described herein can comprise modifications including e.g., conjugation of a palmitoyl group.

As used herein, the term “functional fragment thereof” refers to a fragment or portion of a protein, e.g., α-Galactosidase A protein, that is still capable of one or functions associated with the full protein (e.g., stimulating, modulating, regulating, or modifying an immune response).

The terms “α-Galactosidase A,” “α-Gal A,” and “GAL” are used interchangeably and refer to a protein with enzymatic activity comprising hydrolysis of terminal, non-reducing α-D-galactose residues in α-D-galactosides, including galactose oligosaccharides, galactomannans and galactolipids. In some aspects, α-Gal A comprises the enzyme described by IUBMB Enzyme Nomenclature EC 3.2.1.22 (as described, for example, in Suzuki et al., J. Biol. Chem. 245:781-786 (1970); Wiederschain, G. and Beyer, E. Dokl. Akad. Nauk S.S.S.R. 231:486-488 (1976)). In some aspects, α-Gal A comprises a protein encoded by a nucleic acid comprising the human GLA gene, for example, the human α-Gal A gene defined by GenBank Accession No. NM_000169. In some aspects, α-Gal A comprises a protein comprising the amino acid sequence defined by GenBank Accession No. NP_000160.

In some aspects, GAL can be obtained from a cell endogenously expressing the α-Gal A, or the α-Gal A can be a recombinant human α-Gal A (rhα-Gal A). In some aspects, the rhα-Gal A is a full-length wild-type α-Gal A. In some aspects, the rhα-Gal A comprises a subset of the amino acid residues present in a wild-type α-Gal A, wherein the subset includes the amino acid residues of the wild-type α-Gal A that form the active site for substrate binding and/or substrate reduction. In some aspects, an rhα-Gal A that is a fusion protein comprising the wild-type α-Gal A active site for substrate binding and/or substrate reduction, as well as other amino acid residues that can or may not be present in the wild type α-Gal A.

α-Gal A can be obtained from commercial sources or can be obtained by synthesis techniques known to a person of ordinary skill in the art. The wild-type enzyme can be purified from a recombinant cellular expression system (e.g., mammalian cells such as CHO cells, or insect cells, see e.g., U.S. Pat. Nos. 5,580,757; 6,395,884; 6,458,574; 6,461,609; 6,210,666; 6,083,725), human placenta, or animal milk.

Other synthesis techniques for obtaining α-Gal A suitable for pharmaceutical use can be found, for example, in U.S. Pat. Nos. 7,560,424; 7,396,811; 423,135; 6,534,300; and 6,537,785; U.S. Published Application Nos. 2009/0203575; 2009/0029467; 2008/0299640; 2008/0241118; 2006/0121018; 2005/0244400; 2007/0280925; and 2004/0029779, and International Published Application No. 2005/077093.

In some aspects, the α-Gal A is agalsidase alpha, produced by genetic engineering technology in a human cell line. Agalsidase alpha is available as Replagal®, from Shire Plc. (Dublin, Ireland). In some aspects, the α-Gal A is agalsidase beta, produced by recombinant DNA technology in a Chinese hamster ovary (CHO) cell line. Agalsidase beta is available as Fabrazyme®, from Sanofi Genzyme (Cambridge, Mass.). In some aspects, the α-Gal A is a recombinant human α-Gal A produced in CHO cells transformed with an expression vector encoding the human α-Gal A gene (JCR Pharmaceuticals Co. Ltd, (Japan)), identified as JR-051.

In addition to proteins that comprise an amino acid sequence that is identical to the human α-Gal A proteins described herein, this disclosure also encompasses α-Gal A proteins that are “substantially similar” thereto. Proteins described herein as being “substantially similar” to a reference protein include proteins that retain some structural and functional features of the native proteins yet differ from the native amino acid sequence at one or more amino acid positions (i.e., by amino acid substitutions).

Proteins altered from the native sequence can be prepared by substituting amino acid residues within a native protein and selecting proteins with the desired activity. For example, amino acid residues of an α-Gal A protein can be systematically substituted with other residues and the substituted proteins can then be tested in standard assays for evaluating the effects of such substitutions on the ability of the protein to hydrolyze a terminal, non-reducing α-D-galactose residues in α-D-galactosides, including galactose oligosaccharides, galactomannans and galactolipids, and/or on the ability to treat or prevent Fabry disease.

In some aspects, to retain functional activity, conservative amino acid substitutions are made. As used herein, “conservative amino acid substitutions” refer to substitutions of an amino acid residue with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). In some aspects, a predicted nonessential amino acid residue in an α-Gal A protein is replaced with another amino acid residue from the same side chain family. Methods of identifying nucleotide and amino acid conservative substitutions which do not eliminate antigen binding are well-known in the art (see, e.g., Brummell et al., Biochem. 32: 1180-1187 (1993); Kobayashi et al. Protein Eng. 12(10):879-884 (1999); and Burks et al. Proc. Natl. Acad. Sci. USA 94:412-417 (1997)).

In some aspects, an α-Gal A protein of the disclosure is about 70%, 71%, 72%, 73%, 74%, 75%, 76%, 77%, 78%, 79%, 80%, 81%, 82%, 83%, 84%, 85%, 86%, 87%, 88%, 89% 90%, 91%, 92%, 93%, 94%, 95%, 96%, 97%, 98%, or 99% identical to the amino acid sequence of an α-Gal A protein described herein or known in the art.

The percent identity between two sequences is a function of the number of identical positions shared by the sequences (i.e., % homology=# of identical positions/total # of positions×100), taking into account the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at worldwideweb.gcg.com), using a NWSgapdna.CMP matrix and a gap weight of 40, 50, 60, 70, or 80 and a length weight of 1, 2, 3, 4, 5, or 6. The percent identity between two nucleotide or amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at http://www.gcg.com), using either a Blossum 62 matrix or a PAM250 matrix, and a gap weight of 16, 14, 12, 10, 8, 6, or 4 and a length weight of 1, 2, 3, 4, 5, or 6.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, word length=12 to obtain nucleotide sequences homologous to the nucleic acid molecules described herein. BLAST protein searches can be performed with the XBLAST program, score=50, word length=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See worldwideweb.ncbi.nlm.nih.gov.

An “antibody” (Ab) includes, without limitation, a glycoprotein immunoglobulin which binds specifically to an antigen and comprises at least two heavy (H) chains and two light (L) chains interconnected by disulfide bonds, or an antigen-binding portion thereof. Each H chain comprises a heavy chain variable region (abbreviated herein as V_{H}) and a heavy chain constant region. The heavy chain constant region comprises three constant domains, C_{H1}, C_{H2} and C_{H3}. Each light chain comprises a light chain variable region (abbreviated herein as V_{L}) and a light chain constant region. The light chain constant region is comprises one constant domain, C_{L}. The V_{H} and V_{L} regions can be further subdivided into regions of hypervariability, termed complementarity determining regions (CDRs), interspersed with regions that are more conserved, termed framework regions (FRs). Each V_{H} and V_{L} comprises three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The term “anti-GAL antibody,” for example, includes a full antibody having two heavy chains and two light chains that specifically binds to α-Gal A and antigen-binding portions of the full antibody.

An immunoglobulin can derive from any of the commonly known isotypes, including but not limited to IgA, secretory IgA, IgG and IgM. IgG subclasses are also well known to those in the art and include but are not limited to human IgG1, IgG2, IgG3 and IgG4. “Isotype” refers to the antibody class or subclass (e.g., IgM or IgG1) that is encoded by the heavy chain constant region genes. The term “antibody” includes, by way of example, both naturally occurring and non-naturally occurring antibodies; monoclonal and polyclonal antibodies; chimeric and humanized antibodies; human or nonhuman antibodies; wholly synthetic antibodies; and single chain antibodies. A nonhuman antibody can be humanized by recombinant methods to reduce its immunogenicity in man. Where not expressly stated, and unless the context indicates otherwise, the term “antibody” also includes an antigen-binding fragment or an antigen-binding portion of any of the aforementioned immunoglobulins, and includes a monovalent and a divalent fragment or portion, and a single chain antibody.

An “isolated antibody” refers to an antibody that is substantially free of other antibodies having different antigenic specificities (e.g., an isolated antibody that binds specifically to α-Gal A is substantially free of antibodies that bind specifically to antigens other than α-Gal A). An isolated antibody that binds specifically to α-Gal A can, however, have cross-reactivity to other antigens, such as α-Gal A molecules from different species. Moreover, an isolated antibody can be substantially free of other cellular material and/or chemicals.

An “anti-antigen antibody” refers to an antibody that binds specifically to the antigen. For example, an anti-GAL antibody binds specifically to GAL.

The term “anti-GLA neutralizing antibody,” “anti-GLA NAb,” “anti-drug antibody,” “ADA,” “neutralizing anti-drug antibody,” or “neutralizing ADA,” refers to an antibody that binds and inactivates (neutralizes) α-Gal A enzyme. In some aspects, if the anti-GLA neutralizing antibodies are present, the enzyme replacement therapy is directly inactivated (neutralized) by the anti-GLA neutralizing antibodies in the plasma (Linthorst et al., Kidny Int 66:1589-1595 (2004); Lenders et al., J Allergy Clin Immunol 141:2289-2292.e7 (2018)).

An “antigen-binding portion” of an antibody (also called an “antigen-binding fragment”) refers to one or more fragments of an antibody that retain the ability to bind specifically to the antigen bound by the whole antibody. It has been shown that the antigen-binding function of an antibody can be performed by fragments of a full-length antibody. Examples of binding fragments encompassed within the term “antigen-binding portion” of an antibody, e.g., an anti-GLA 3 antibody described herein, include (i) a Fab fragment (fragment from papain cleavage) or a similar monovalent fragment consisting of the V_{L}, V_{H}, LC and CH1 domains; (ii) a F(ab′)2 fragment (fragment from pepsin cleavage) or a similar bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a dAb fragment (Ward et al., (1989) Nature 341:544-546), which consists of a V_{H} domain; (vi) an isolated complementarity determining region (CDR) and (vii) a combination of two or more isolated CDRs which can optionally be joined by a synthetic linker. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see, e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies. Antigen-binding portions can be produced by recombinant DNA techniques, or by enzymatic or chemical cleavage of intact immunoglobulins.

“Binding affinity” generally refers to the strength of the sum total of non-covalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured and/or expressed in a number of ways known in the art, including, but not limited to, equilibrium dissociation constant (K_{D}), and equilibrium association constant (K_{A}). The K_{D} is calculated from the quotient of k_{off}/kₒₙ and is expressed as a molar concentration (M), whereas K_{A} is calculated from the quotient of kₒₙ/k_{off}. kₒₙ refers to the association rate constant of, e.g., an antibody to an antigen, and k_{off} refers to the dissociation of, e.g., an antibody to an antigen. The kₒₙ and k_{off} can be determined by techniques known to one of ordinary skill in the art, such as immunoassays (e.g., enzyme-linked immunosorbent assay (ELISA)), BIACORE®, BLI (Bio-layer interferometry), or kinetic exclusion assay (KINEXA®).

As used herein, the terms “specifically binds,” “specifically recognizes,” “specific binding,” “selective binding,” and “selectively binds,” are analogous terms in the context of antibodies and refer to molecules (e.g., antibodies) that bind to an antigen (e.g., epitope or immune complex) as such binding is understood by one skilled in the art. For example, a molecule that specifically binds to an antigen can bind to other peptides or polypeptides, generally with lower affinity as determined by, e.g., immunoassays, BIACORE®, KINEXA® 3000 instrument (Sapidyne Instruments, Boise, ID), or other assays known in the art. In a specific aspect, molecules that specifically bind to an antigen bind to the antigen with a K_{A} that is at least 2 logs, 2.5 logs, 3 logs, 4 logs or greater than the K_{A} when the molecules bind to another antigen.

Antibodies typically bind specifically to their cognate antigen with high affinity, reflected by a dissociation constant (K_{D}) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸ M or less, even more preferably 10⁻⁹ M or less, and most preferably between 10⁻⁸ M and 10⁻¹⁰ M or less, when determined by, e.g., immunoassays (e.g., ELISA) surface plasmon resonance (SPR) technology in a BIACORE™ 2000 instrument using the predetermined antigen, or BLI (Bio-layer interferometry) but does not bind with high affinity to unrelated antigens.

The term “recombinant host cell” (or simply “host cell”), as used herein, is intended to refer to a cell that comprises a nucleic acid that is not naturally present in the cell, and can be a cell into which a recombinant expression vector has been introduced. It should be understood that such terms are intended to refer not only to the particular subject cell but to the progeny of such a cell. Because some modifications can occur in succeeding generations due to either mutation or environmental influences, such progeny cannot, in fact, be identical to the parent cell, but are still included within the scope of the term “host cell” as used herein.

As used herein, the term “linked” refers to the association of two or more molecules. The linkage can be covalent or non-covalent. The linkage also can be genetic (i.e., recombinantly fused). Such linkages can be achieved using a wide variety of art recognized techniques, such as chemical conjugation and recombinant protein production.

The term “Fabry disease” refers to classical Fabry disease, late-onset Fabry disease, and hemizygous females having mutations in the gene encoding an α-Gal A. The term “Fabry disease,” as used herein, further includes any condition in which a subject exhibits lower than normal endogenous α-Gal A activity. Fabry disease is referred to by many other names, for example, alpha-galactosidase A deficiency, Anderson-Fabry disease, angiokeratoma corporis diffusum, angiokeratoma diffuse, ceramide trihexosidase deficiency, Fabry's disease, GLA deficienc, and hereditary dystopic lipidosis. In some aspects, Fabry disease is type 1 classic phenotype or type 2 later-onset phenotype.

The term “enzyme replacement therapy” or “ERT” refers to the introduction of a non-native, purified enzyme into an individual having a deficiency in such enzyme (e.g., α-Gal A). The administered enzyme can be obtained from natural sources or by recombinant expression. The term also refers to the introduction of a purified enzyme in an individual otherwise requiring or benefiting from administration of a purified enzyme, e.g., suffering from protein insufficiency. The introduced enzyme can be a purified, recombinant enzyme produced in vitro, or enzyme purified from isolated tissue or fluid, such as, e.g., placenta or animal milk, or from plants.

The term “co-formulation” refers to a composition comprising an enzyme, such as an enzyme used for ERT (e.g., a human recombinant α-Gal A enzyme (rhα-Gal A)), that is formulated together with an Active Site-Specific Chaperone (ASSC) for the α-Gal A enzyme (e.g., 1-deoxygalactonojirimycin (DGJ)). In some aspects, the ASSC is 1-deoxygalactonojirimycin (DGJ), or a pharmaceutically acceptable salt, ester or prodrug of 1-deoxygalactonojirimycin. In some aspects, the salt is hydrochloride salt (i.e. 1-deoxygalactonojirimycin-HCl). In some aspects, treating a subject with the co-formulation comprises administering the co-formulation to the subject such that the α-Gal A enzyme and ASSC are administered concurrently at the same time as part of the co-formulation.

The term “combination therapy” refers to any therapy wherein the results are enhanced as compared to the effect of each therapy when it is performed individually. The individual therapies in a combination therapy can be administered concurrently or consecutively.

Enhancement can include any improvement of the effect of the various therapies that can result in an advantageous result as compared to the results achieved by the therapies when performed alone. Enhanced effect and determination of enhanced effect can be measured by various parameters such as, but not limited to: temporal parameters (e.g., length of treatment, recovery time, long-term effect of the treatment or reversibility of treatment); biological parameters (e.g., cell number, cell volume, cell composition, tissue volume, tissue size, tissue composition); spatial parameters (e.g., tissue strength, tissue size or tissue accessibility) and physiological parameters (e.g., body contouring, pain, discomfort, recovery time or visible marks). Enhanced effect can include a synergistic enhancement, wherein the enhanced effect is more than the additive effects of each therapy when performed by itself. Enhanced effect can also include an additive enhancement, wherein the enhanced effect is substantially equal to the additive effect of each therapy when performed by itself. Enhanced effect can also include less than a synergistic effect, wherein the enhanced effect is lower than the additive effect of each therapy when performed by itself, but still better than the effect of each therapy when performed by itself.

The term “stabilize a proper conformation” refers to the ability of a compound or peptide or other molecule to associate with a wild-type protein, or to a mutant protein that can perform its wild-type function in vitro and in vivo, in such a way that the structure of the wild-type or mutant protein can be maintained as its native or proper form. This effect can manifest itself practically through one or more of (i) increased shelf-life of the protein; (ii) higher activity per unit/amount of protein; or (iii) greater in vivo efficacy. It can be observed experimentally through increased yield from the ER during expression; greater resistance to unfolding due to temperature increases (e.g., as determined in thermal stability assays), or the present of chaotropic agents, and by similar means.

As used herein, the term “active site” refers to the region of a protein that has some specific biological activity. For example, it can be a site that binds a substrate or other binding partner and contributes the amino acid residues that directly participate in the making and breaking of chemical bonds. Active sites in this application can encompass catalytic sites of enzymes, antigen biding sites of antibodies, ligand binding domains of receptors, binding domains of regulators, or receptor binding domains of secreted proteins. The active sites can also encompass transactivation, protein-protein interaction, or DNA binding domains of transcription factors and regulators.

As used herein, the term “active site-specific chaperone” refers to any molecule including a protein, peptide, nucleic acid, carbohydrate, etc. that specifically interacts reversibly with an active site of a protein and enhances formation of a stable molecular conformation. As used herein, “active site-specific chaperone” does not include endogenous general chaperones present in the ER of cells such as Bip, calnexin or calreticulin, or general, non-specific chemical chaperones such as deuterated water, DMSO, or TMAO.

The term “non-enzyme replacement therapy” refers to a therapy (e.g., Fabry disease therapy) that is not an enzyme replacement therapy. The non-enzyme replacement therapy can include small molecule therapy. Some emerging drug development strategies for small molecule therapy of Fabry disease include but are not limited to substrate reduction therapy (SRT), residual enzyme activation, GLA promoter activation, protein homeostasis regulation (proteostasis), and chemical chaperone therapy (CCT).

The term “immunotherapy” refers to the treatment of a subject afflicted with, or at risk of contracting or suffering a recurrence of, a disease by a method comprising inducing, enhancing, suppressing or otherwise modifying an immune response. “Treatment” or “therapy” of a subject refers to any type of intervention or process performed on, or the administration of an active agent to, the subject with the objective of reversing, alleviating, ameliorating, inhibiting, slowing down or preventing the onset, progression, development, severity or recurrence of a symptom, complication or condition, or biochemical indicia associated with a disease (e.g., Fabry disease). “Immunosuppressive therapy” refers to a therapy that results in decreasing (reducing) an immune response in a subject.

The phrase “contacting a cell” (e.g., contacting a cell with an AAV expression vector or the composition of the disclosure, as used herein, includes contacting a cell directly or indirectly. In some aspects, contacting a cell with an AAV expression vector or the composition of the present disclosure includes contacting a cell in vitro with the composition or the AAV vector or contacting a cell in vivo with the AAV vector or the composition of the present disclosure. Thus, for example, the AAV vector or the composition of the present disclosure can be put into physical contact with the cell by the individual performing the method, or alternatively, the AAV vector or the composition of the present disclosure can be put into a situation that will permit or cause it to subsequently come into contact with the cell.

In some aspects, contacting a cell in vitro can be done, for example, by incubating the cell with the AAV vector. In some aspects, contacting a cell in vivo can be done, for example, by injecting the AAV vector or the composition of the disclosure into or near the tissue where the cell is located, or by injecting the AAV vector or the composition of the present disclosure into another area, e.g., the bloodstream or the subcutaneous space, such that the agent will subsequently reach the tissue where the cell to be contacted is located. For example, the AAV vector can be encapsulated and/or coupled to a ligand that directs the AAV vector to a site of interest. Combinations of in vitro and in vivo methods of contacting are also possible. For example, a cell can be contacted in vitro with an AAV vector or the composition of the present disclosure and subsequently transplanted into a subject.

In some aspects, contacting a cell with an AAV vector or the composition of the present disclosure includes “introducing” or “delivering” (directly or indirectly) the AAV vector or the composition of the present disclosure into the cell by facilitating or effecting uptake or absorption into the cell. Introducing an AAV vector or the composition of the present disclosure into a cell can be in vitro and/or in vivo. For example, for in vivo introduction, an AAV vector or the composition of the present disclosure can be injected into a specific tissue site (e.g., the locus where a therapeutic effect is desired) or administered systemically (e.g., administering a AAV vector targeted to a locus where a therapeutic effect is desired). In vitro introduction into a cell includes methods known in the art such as electroporation and lipofection.

As used herein, the terms “effective amount,” “therapeutically effective amount,” and a “sufficient amount” of, e.g., an AAV vector or the composition disclosed herein refer to a quantity sufficient to, when administered to the subject, including a human, effect beneficial or desired results, including clinical results, and, as such, an “effective amount” or synonym thereto depends on the context in which it is being applied. In some aspects, a therapeutically effective amount of an agent (e.g., an AAV vector or the composition disclosed herein) is an amount that results in a beneficial or desired result in a subject as compared to a control.

The amount of a given agent (e.g., an AAV vector or the composition disclosed herein) will correspond to such an amount will vary depending upon various factors, such as the given agent, the pharmaceutical formulation, the route of administration, the type of disease or disorder, the identity of the subject (e.g., age, sex, and/or weight) or host being treated, and the like.

As used herein, the term “gene therapy” is the insertion of nucleic acid sequences (e.g., a polynucleotide comprising a promoter operably linked to a nucleic acid encoding an immunomodulatory protein (e.g., a cytokine or subunit thereof) or functional fragment thereof as disclosed herein) into an individual's cells and/or tissues to treat, reduce the symptoms of, or reduce the likelihood of a disease. Gene therapy also includes insertion of transgene that are inhibitory in nature, i.e., that inhibit, decrease or reduce expression, activity or function of an endogenous gene or protein, such as an undesirable or aberrant (e.g., pathogenic) gene or protein. Such transgenes can be exogenous. An exogenous molecule or sequence is understood to be molecule or sequence not normally occurring in the cell, tissue and/or individual to be treated. Both acquired and congenital diseases are amenable to gene therapy.

The term “prophylactically effective amount,” as used herein, includes the amount of an agent, (e.g., an AAV vector or the composition disclosed herein) that, when administered to a subject having or predisposed to have a disease or disorder (e.g., Fabry disease), is sufficient to prevent, reduce the symptoms of, or ameliorate the disease or disorder or one or more symptoms of the disease or disorder. Ameliorating the disease or disorder includes slowing the course of the disease or disorder or reducing the severity of later-developing disease or disorder. The “prophylactically effective amount” can vary depending on the characteristics of the agent, e.g., an AAV expression vector or the composition of the present disclosure, how the agent is administered, the degree of risk of disease, and the history, age, weight, family history, genetic makeup, the types of preceding or concomitant treatments, if any, and other individual characteristics of the patient to be treated.

As used herein, “off target” refers to any unintended effect on any one or more target, gene, or cellular transcript.

As used herein, the term “in vitro” refers to events that occur in an artificial environment, e.g., in a test tube or reaction vessel, in cell culture, in a Petri dish, etc., rather than within an organism (e.g., animal, plant, or microbe).

As used herein, the term “in vivo” refers to events that occur within an organism (e.g., animal, plant, or microbe or cell or tissue thereof).

As used herein, the term “transfection” refers to methods to introduce exogenous nucleic acids into a cell. Methods of transfection include, but are not limited to, chemical methods, physical treatments and cationic lipids or mixtures. The list of agents that can be transfected into a cell is large and includes, e.g., siRNA, shRNA, sense and/or anti-sense sequences, DNA encoding one or more genes and organized into an expression plasmid, e.g., a vector.

By “determining the level of a protein” is meant the detection of a protein, or an mRNA encoding the protein, by methods known in the art either directly or indirectly. “Directly determining” means performing a process (e.g., performing an assay or test on a sample or “analyzing a sample” as that term is defined herein) to obtain the physical entity or value. “Indirectly determining” refers to receiving the physical entity or value from another party or source (e.g., a third-party laboratory that directly acquired the physical entity or value). Methods to measure protein level generally include, but are not limited to, western blotting, immunoblotting, enzyme-linked immunosorbent assay (ELISA), radioimmunoassay (RIA), immunoprecipitation, immunofluorescence, surface plasmon resonance, chemiluminescence, fluorescent polarization, phosphorescence, immunohistochemical analysis, matrix-assisted laser desorption/ionization time-of-flight (MALDI-TOF) mass spectrometry, liquid chromatography (LC)-mass spectrometry, microcytometry, microscopy, fluorescence activated cell sorting (FACS), and flow cytometry, as well as assays based on a property of a protein including, but not limited to, enzymatic activity or interaction with other protein partners. Methods to measure mRNA levels are known in the art.

The term “level,” as used herein, refers to a level/amount or activity of a protein, or mRNA encoding the protein, optionally as compared to a reference. The reference can be any useful reference, as defined herein. A level of a protein can be expressed in mass/vol (e.g., g/dL, mg/mL, μg/mL, ng/ml) or percentage relative to total protein or mRNA in a sample.

The term “decreased level” or a “reduced level” of a protein, as used herein, means a decrease/reduction in protein level, as compared to a reference. The term “reducing the amount of glycosphingolipids” in a subject, as used herein, means a decrease/reduction in the amount (ng/ml) of glycosphingolipids (e.g., globotriaosylceramide (Gb3), globotriaosylsphingosine (lyso-Gb3), galabiosylceramide, or any combination thereof) relative to the amount (ng/ml) of glycosphingolipids in the same subject prior to the administration. The decrease/reduction in the amount (ng/ml) of glycosphingolipids is measured from a baseline point to the last measured point as described herein in Example 2 below. In some aspects, the amount of glycosphingolipids (e.g., lyso-Gb3 concentration (ng/ml)) is measured from a baseline point to the last measured point as described herein in Example 2 below. In some aspects, the amount of glycosphingolipids (e.g., Gb3 concentration (ng/ml)) is measured from a baseline point to the last measured point as described herein in Example 2 below. The term “baseline point,” as used herein refers the time point immediately preceding administration of the AAV expression vector or pharmaceutical composition of the present disclosure.

The term “increased level” of a protein, as used herein, means an increase in protein level, as compared to a reference. The term “increasing an α galactosidase A (α-Gal A) protein activity” in a subject, as used herein, means an increase in α-Gal A protein (enzyme) activity (nmol/h/ml) relative to the α-Gal A protein activity in the subject prior to the administration, where the increase in α-Gal A protein activity is measured as described herein in Example 2 below.

The term a “maintained level” of a protein, as used herein, means no significant decrease/reduction or increase in protein level, as compared to a reference. The term “maintains the amount of glycosphingolipids” in a subject, as used herein, means that there is no statistically significant decrease/reduction or increase in the amount of glycosphingolipids (e.g., globotriaosylceramide (Gb3), globotriaosylsphingosine (lyso-Gb3), galabiosylceramide, or any combination thereof) relative to the amount of glycosphingolipids in the same subject prior to the administration. In some aspects, the subject is an enzyme replacement therapy (ERT) pre-treated subject. ERT must have been administered at a stable dose (defined as not having missed more than 3 doses of ERT during the 6 months prior to consent) and regimen (14 days±1 day for at least 3 months prior to enrollment). Whether a part is statistically significant can be determined in a simple manner by the person skilled in the art using various well known statistical evaluation tools, for example, the determination of confidence intervals, determination of p values, Student's T test, Mann-Whitney test, etc. Details are provided in Dowdy and Wearden, Statistics for Research, John Wiley and Sons, New York 1983. The preferred confidence intervals are at least about 90%, at least about 95%, at least about 97%, at least 98% or at least 99%. The p values are preferably 0.1, 0.05, 0.01, 0.005 or 0.0001.

The term “pharmaceutical composition,” as used herein, represents a composition comprising a compound or molecule described herein, e.g., an AAV vector disclosed herein, formulated with a pharmaceutically acceptable excipient, and can be manufactured or sold with the approval of a governmental regulatory agency as part of a therapeutic regimen for the treatment of disease in a mammal.

A “pharmaceutically acceptable excipient,” as used herein, refers to any ingredient other than the compounds described herein (for example, a vehicle capable of suspending or dissolving the active compound) and having the properties of being substantially nontoxic and non-inflammatory in a patient.

By a “reference” is meant any useful reference used to compare protein or mRNA levels or activity. The reference can be any sample, standard, standard curve, or level that is used for comparison purposes. The reference can be a normal reference sample or a reference standard or level. A “reference sample” can be, for example, a control, e.g., a predetermined negative control value such as a “normal control” or a prior sample taken from the same subject; a sample from a normal healthy subject, such as a normal cell or normal tissue; a sample (e.g., a cell or tissue) from a subject not having a disease; a sample from a subject that is diagnosed with a disease, but not yet treated with a compound described herein; a sample from a subject that has been treated by a compound described herein; or a sample of a purified protein (e.g., any described herein) at a known normal concentration.

As used herein, the term “subject” refers to any organism to which a composition disclosed herein, e.g., an AAV expression vector or the composition of the present disclosure, can be administered, e.g., for experimental, diagnostic, prophylactic, and/or therapeutic purposes. Typical subjects include any animal (e.g., mammals such as mice, rats, rabbits, non-human primates, and humans). A subject can seek or be in need of treatment, require treatment, be receiving treatment, be receiving treatment in the future, or be a human or animal who is under care by a trained professional for a particular disease or condition. In some aspects, the subject is a human. The terms, “subject” and “patient” are used interchangeably herein.

As used herein, the term “comparable untreated subject with Fabry disease” refers to a human subject matched with treated subject, for example, by age, sex, race, and/or disease manifestations. (See e.g., Giugliani et al., Journal of Inborn Errors of Metabolism & Screening Volume 4:1-12 (2016)).

The term “vector genomes per kilogram of body weight (vg/kg)” as used herein, refers to copies of the expression cassette (e.g., the α-Gal A expression cassette of the present disclosure) administered into a subject. The number of vector genome copies per kilogram of body weight can be measured by quantitative PCR and/or droplet digital PCR.

The use of the term “flat dose” with regard to the methods and dosages of the disclosure means a dose that is administered to a patient without regard for the weight or body surface area (BSA) of the patient. The flat dose is therefore not provided as a mg/kg dose, but rather as an absolute amount of the agent (e.g., recombinant α-Gal A protein). For example, a 60 kg person and a 100 kg person would receive the same dose of an antibody (e.g., 12 mg of recombinant α-Gal A protein).

The term “weight-based dose” as referred to herein means that a dose that is administered to a patient is calculated based on the weight of the patient. For example, when a patient with 60 kg body weight requires 0.2 mg/kg of recombinant α-Gal A protein, one can calculate and use the appropriate amount of recombinant α-Gal A protein (i.e., 12 mg) for administration.

A “therapeutically effective amount” or “therapeutically effective dosage” of a drug or therapeutic agent is any amount of the drug that, when used alone or in combination with another therapeutic agent, protects a subject against the onset of a disease or promotes disease regression evidenced by a decrease in severity of disease symptoms, an increase in frequency and duration of disease symptom-free periods, or a prevention of impairment or disability due to the disease affliction. The ability of a therapeutic agent to promote disease regression can be evaluated using a variety of methods known to the skilled practitioner, such as in human subjects during clinical trials, in animal model systems predictive of efficacy in humans, or by assaying the activity of the agent in in vitro assays.

The terms “treat,” “treating,” and “treatment,” as used herein, refer to any type of intervention or process performed on, or administering an active agent to, the subject with the objective of reversing, alleviating, ameliorating, inhibiting, or slowing down or preventing the progression, development, severity or recurrence of a symptom, complication, condition or biochemical indicia associated with a disease or enhancing overall survival. Treatment can be of a subject having a disease or a subject who does not have a disease (e.g., for prophylaxis).

As used herein, the term “preventing” when used in relation to a condition, refers to administration of a composition which reduces the frequency of, or delays the onset of, symptoms of a medical condition in a subject relative to a subject which does not receive the composition.

The term “ameliorating” refers to any therapeutically beneficial result in the treatment of a disease state, e.g., Fabry disease, including prophylaxis, lessening in the severity or progression, remission, or cure thereof. In some aspects, the methods of the present disclosure are ameliorating one or more symptoms associated with Fabry disease in a human subject in need thereof.

The term “effective dose” or “effective dosage” is defined as an amount sufficient to achieve or at least partially achieve a desired effect. A “therapeutically effective amount” or “therapeutically effective dosage” of a drug or therapeutic agent is any amount of the drug that, when used alone or in combination with another therapeutic agent, promotes disease regression evidenced by a decrease in severity of disease symptoms, an increase in frequency and duration of disease symptom-free periods, an increase in overall survival (the length of time from either the date of diagnosis or the start of treatment for a disease, such as Fabry disease, that patients diagnosed with the disease are still alive), or a prevention of impairment or disability due to the disease affliction. A therapeutically effective amount or dosage of a drug includes a “prophylactically effective amount” or a “prophylactically effective dosage”, which is any amount of the drug that, when administered alone or in combination with another therapeutic agent to a subject at risk of developing a disease or of suffering a recurrence of disease, inhibits the development or recurrence of the disease. The ability of a therapeutic agent to promote disease regression or inhibit the development or recurrence of the disease can be evaluated using a variety of methods known to the skilled practitioner, such as in human subjects during clinical trials, in animal model systems predictive of efficacy in humans, or by assaying the activity of the agent in in vitro assays.

A “sample” or “biological sample” of the disclosure is of biological origin, in some aspects, such as from eukaryotic organisms. In some aspects, the sample is a human sample, but animal samples can also be used. Non-limiting sources of a sample for use in the present disclosure include solid tissue, biopsy aspirates, ascites, fluidic extracts, blood, plasma, serum, spinal fluid, lymph fluid, the external sections of the skin, respiratory, intestinal, and genitourinary tracts, tears, saliva, milk, tumors, organs, cell cultures and/or cell culture constituents, for example.

“Administering” refers to the physical introduction of a composition comprising a therapeutic agent to a subject, using any of the various methods and delivery systems known to those skilled in the art. Preferred routes of administration for recombinant α-Gal A protein or a gene expressing α-Gal A, include intravenous or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. Other non-parenteral routes include an oral, topical, epidermal or mucosal route of administration, for example, intranasally, vaginally, rectally, sublingually or topically. In some aspects, the AAV expression vector of the present disclosure is administered intravenously.

Administering can also be performed, for example, once, a plurality of times, and/or over one or more extended periods. In some aspects, the AAV expression vector of the disclosure is administered at only one dose.

The terms “once about every week,” “once about every two weeks,” or any other similar dosing interval terms as used herein mean approximate numbers. “Once about every week” can include every seven days±one day, i.e., every six days to every eight days. “Once about every two weeks” can include every fourteen days±three days, i.e., every eleven days to every seventeen days. Similar approximations apply, for example, to once about every three weeks, once about every four weeks, once about every five weeks, once about every six weeks, and once about every twelve weeks. In some aspects, a dosing interval of once about every six weeks or once about every twelve weeks means that the first dose can be administered any day in the first week, and then the next dose can be administered any day in the sixth or twelfth week, respectively. In other aspects, a dosing interval of once about every six weeks or once about every twelve weeks means that the first dose is administered on a particular day of the first week (e.g., Monday) and then the next dose is administered on the same day of the sixth or twelfth weeks (i.e., Monday), respectively.

The term “comprising essentially of” refers to a value or composition that is within an acceptable error range for the particular value or composition as determined by one of ordinary skill in the art, which will depend in part on how the value or composition is measured or determined, i.e., the limitations of the measurement system. For example, “comprising essentially of” can mean within 1 or more than 1 standard deviation per the practice in the art. Alternatively, “comprising essentially of” can mean a range of up to 10%. Furthermore, particularly with respect to biological systems or processes, the terms can mean up to an order of magnitude or up to 5-fold of a value. When particular values or compositions are provided in the application and claims, unless otherwise stated, the meaning of “comprising essentially of” should be assumed to be within an acceptable error range for that particular value or composition.

Unless defined otherwise, all technical and scientific terms used herein have the same meaning as commonly understood by one of ordinary skill in the art to which this disclosure is related. For example, the Concise Dictionary of Biomedicine and Molecular Biology, Juo, Pei-Show, 2nd ed., 2002, CRC Press; The Dictionary of Cell and Molecular Biology, 3rd ed., 1999, Academic Press; and the Oxford Dictionary of Biochemistry And Molecular Biology, Revised, 2000, Oxford University Press, provide one of skill with a general dictionary of many of the terms used in this disclosure.

Units, prefixes, and symbols are denoted in their Système International de Unites (SI) accepted form. Numeric ranges are inclusive of the numbers defining the range. Unless otherwise indicated, nucleotide sequences are written left to right in 5′ to 3′ orientation. Amino acid sequences are written left to right in amino to carboxy orientation. The headings provided herein are not limitations of the various aspects of the disclosure, which can be had by reference to the specification as a whole. Accordingly, the terms defined immediately below are more fully defined by reference to the specification in its entirety. As described herein, any concentration range, percentage range, ratio range or integer range is to be understood to include the value of any integer within the recited range and, when appropriate, fractions thereof (such as one tenth and one hundredth of an integer), unless otherwise indicated.

Various aspects of the disclosure are described in further detail in the following subsections.

### II. Methods of the Disclosure

Provided herein are the methods for treating or ameliorating one or more symptoms of Fabry disease, reducing the amount of glycosphingolipids, and/or increasing an α galactosidase A (α-Gal A) protein activity in a subject in need thereof by administering a therapeutically effective amount of the expression vectors (e.g., an adeno-associated virus (AAV) expression vector) comprising an α galactosidase A (α-Gal A) expression cassette, which comprises an α galactosidase A (α-Gal A) transgene encoding the at least one α-Gal A protein.

In some aspects, the disclosure is directed to a method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg.

In some aspects, the disclosure is directed to a method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg.

In some aspects, the disclosure is directed to a method of reducing the amount of glycosphingolipids in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

In some aspects, the disclosure is directed to a method of reducing the amount of glycosphingolipids in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

In some aspects, the disclosure is directed to a method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α-Gal A transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

In some aspects, the disclosure is directed to a method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an α-Gal A transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

In some aspects, the AAV expression vector of the disclosure is administered at a dose of about 1×10¹¹ vg/kg, about 2×10¹¹ vg/kg, about 3×10¹¹ vg/kg, about 4×10¹¹ vg/kg, about 5×10¹¹ vg/kg, about 6×10¹¹ vg/kg, about 7×10¹¹ vg/kg, about 8×10¹¹ vg/kg, about 9×10¹¹ vg/kg, about 1×10¹² vg/kg, about 2×10¹² vg/kg, about 3×10¹² vg/kg, about 4×10¹² vg/kg, about 5×10¹² vg/kg, about 6×10¹² vg/kg, about 7×10¹² vg/kg, about 8×10¹² vg/kg, about 9×10¹² vg/kg, about 1×10¹³ vg/kg, about 2×10¹³ vg/kg, about 3×10¹³ vg/kg, about 4×10¹³ vg/kg, about 5×10¹³ vg/kg, about 6×10¹³ vg/kg, about 7×10¹³ vg/kg, about 8×10¹³ vg/kg, about 9×10¹³ vg/kg, about 1×10¹⁴ vg/kg, about 2×10¹⁴ vg/kg, about 3×10¹⁴ vg/kg, about 4×10¹⁴ vg/kg, about 5×10¹⁴ vg/kg, about 6×10¹⁴ vg/kg, about 7×10¹⁴ vg/kg, about 8×10¹⁴ vg/kg, or about 9×10¹⁴ vg/kg.

In some aspects, the AAV expression vector of the disclosure is administered at a dose of about 5×10¹² vg/kg. In some aspects, the AAV expression vector of the disclosure is administered at a dose of about 1×10¹³ vg/kg. In some aspects, the AAV expression vector of the disclosure is administered at a dose of about 3×10¹³ vg/kg. In some aspects, the AAV expression vector of the disclosure is administered at a dose of about 5×10¹³ vg/kg.

In some aspects, the AAV expression vector of the disclosure is administered at only one dose of about 1×10¹¹ vg/kg. In some aspects, the AAV expression vector of the disclosure is administered at only one dose of about 2×10¹¹ vg/kg. In some aspects, the AAV expression vector of the disclosure is administered at only one dose of about 3×10¹¹ vg/kg. In some aspects, the AAV expression vector of the disclosure is administered at only one dose of about 4×10¹¹ vg/kg. In some aspects, the AAV expression vector of the disclosure is administered at only one dose of about 5×10¹¹ vg/kg. In some aspects, the AAV expression vector of the disclosure is administered at only one dose of about 6×10¹¹ vg/kg. In some aspects, the AAV expression vector of the disclosure is administered at only one dose of about 7×10¹¹ vg/kg. In some aspects, the AAV expression vector of the disclosure is administered at only one dose of about 8×10¹¹ vg/kg. In some aspects, the AAV expression vector of the disclosure is administered at only one dose of about 9×10¹¹ vg/kg. In some aspects, the AAV expression vector of the disclosure is administered at only one dose of about 1×10¹² vg/kg. In some aspects, the AAV expression vector of the disclosure is administered at only one dose of about 2×10¹² vg/kg. In some aspects, the AAV expression vector of the disclosure is administered at only one dose of about 3×10¹² vg/kg. In some aspects, the AAV expression vector of the disclosure is administered at only one dose of about 4×10¹² vg/kg. In some aspects, the AAV expression vector of the disclosure is administered at only one dose of about 5×10¹² vg/kg. In some aspects, the AAV expression vector of the disclosure is administered at only one dose of about 6×10¹² vg/kg. In some aspects, the AAV expression vector of the disclosure is administered at only one dose of about 7×10¹² vg/kg, about 8×10¹² vg/kg. In some aspects, the AAV expression vector of the disclosure is administered at only one dose of about 9×10¹² vg/kg. In some aspects, the AAV expression vector of the disclosure is administered at only one dose of about 1×10¹³ vg/kg. In some aspects, the AAV expression vector of the disclosure is administered at only one dose of about 2×10¹³ vg/kg. In some aspects, the AAV expression vector of the disclosure is administered at only one dose of about 3×10¹³ vg/kg. In some aspects, the AAV expression vector of the disclosure is administered at only one dose of about 4×10¹³ vg/kg. In some aspects, the AAV expression vector of the disclosure is administered at only one dose of about 5×10¹³ vg/kg. In some aspects, the AAV expression vector of the disclosure is administered at only one dose of about 6×10¹³ vg/kg. In some aspects, the AAV expression vector of the disclosure is administered at only one dose of about 7×10¹³ vg/kg. In some aspects, the AAV expression vector of the disclosure is administered at only one dose of about 8×10¹³ vg/kg. In some aspects, the AAV expression vector of the disclosure is administered at only one dose of about 9×10¹³ vg/kg. In some aspects, the AAV expression vector of the disclosure is administered at only one dose of about 1×10¹⁴ vg/kg. In some aspects, the AAV expression vector of the disclosure is administered at only one dose of about 2×10¹⁴ vg/kg. In some aspects, the AAV expression vector of the disclosure is administered at only one dose of about 3×10¹⁴ vg/kg. In some aspects, the AAV expression vector of the disclosure is administered at only one dose of about 4×10¹⁴ vg/kg. In some aspects, the AAV expression vector of the disclosure is administered at only one dose of about 5×10¹⁴ vg/kg. In some aspects, the AAV expression vector of the disclosure is administered at only one dose of about 6×10¹⁴ vg/kg. In some aspects, the AAV expression vector of the disclosure is administered at only one dose of about 7×10¹⁴ vg/kg. In some aspects, the AAV expression vector of the disclosure is administered at only one dose of about 8×10¹⁴ vg/kg. In some aspects, the AAV expression vector of the disclosure is administered at only one dose of about 9×10¹⁴ vg/kg.

In some aspects, the AAV expression vector of the disclosure is administered at only one dose of about 5×10¹² vg/kg. In some aspects, the AAV expression vector of the disclosure is administered at only one dose of about 1×10¹³ vg/kg. In some aspects, the AAV expression vector of the disclosure is administered at only one dose of about 3×10¹³ vg/kg. In some aspects, the AAV expression vector of the disclosure is administered at only one dose of about 5×10¹³ vg/kg.

In some aspects, the AAV expression vectors disclosed herein are administered to a subject, e.g., a human subject, using a variety of methods that depend, in part, on the route of administration. In some aspects, the AAV expression vector of the present disclosure is administered parenterally. In some aspects, the AAV expression vector of the present disclosure is administered intravenously. In some aspects, the AAV expression vector disclosed herein is administered to the subject by intravenous infusion.

In some aspects, the subject has Fabry disease. In some aspects, the subject has one or more of the following symptoms associated with Fabry disease: globotriaosylceramide (Gb3) levels above normal, globotriaosylsphingosine (lyso-Gb3) levels above normal, renal disease, cardiac disease, anhidrosis, acroparesthesia, angiokeratoma, gastrointestinal (GI) tract pain, corneal and lenticular opacities, or cerebrovascular disease. In some aspects, angiokeratoma is periumbilical angiokeratoma. In some aspects, Fabry disease is type 1 classic phenotype or type 2 later-onset phenotype.

In some aspects, the subject has the α-GalA protein activity of less than about 5%. In some aspects, the subject is an enzyme replacement therapy (ERT) naïve subject. In some aspects, the ERT naïve subject is ERT naïve classic Fabry male.

In some aspects, the α-GalA protein activity is measured in the subject's plasma skin, and/or leukocytes as described herein in Example 2 below.

In some aspects, the subject is a male subject. In some aspects, the subject is a female subject.

In some aspects, the subject has an α-GalA gene mutation that is indicative of Fabry disease (e.g., classical Fabry disease) as, for example, listed in a database, such as International Fabry Disease Genotype-Phenotype Database (dbFGP). In some aspects, the α-Gal A gene mutation can result, but it is not limited to the amino acid mutation G261D, C422T, W340R, S297Y, Q283X, D215S, IVS5/c.801+3A>G, P362L, C422T, or N34S.

In some aspects, the subject has pre-existing anti-α-GalA antibodies prior to the administering as determined by an enzyme-linked immunosorbent assay (ELISA). The term “pre-existing anti-α-GalA antibodies,” as used herein refers to the total anti-drug antibodies (ADA) against α-GalA in e.g., human serum, wherein the ADA antibodies are detected using an enzyme-linked immunosorbent assay (ELISA). Sample with % inhibition ≥46.1 is reported as positive for the presence of antibodies against α-GalA (“anti-α-Gal A antibody positive”).

### Anti-GLA Neutralizing Antibody

The total anti-drug antibodies (ADA) against α-GalA in human serum are detected and titrated in human serum in a fluorescent enzyme inhibition assay. The assay determines the presence of anti-α-GalA neutralizing antibodies (NAb) by assessing the neutralizing capacity of human serum on the α-GalA activity. Samples having % inhibition equal to or greater than the cutoff point of 9.6% are identified as NAb positive while those below the cut point are considered negative.

In some aspects, the subject is an anti-α-GalA neutralizing antibody positive subject when a biological sample of the subject is analyzed, wherein the anti-α-GalA neutralizing antibody positive subject has a biological sample having greater than about 9.6% inhibition of α-Galactosidase A activity as measured by an anti-α-GalA neutralizing antibody assay described above.

In some aspects, the anti-GLA neutralizing antibody binds and inactivates (neutralizes) α-Gal A enzyme. In some aspects, if the anti-GLA neutralizing antibodies are present, the enzyme replacement therapy is directly inactivated (neutralized) by the anti-GLA neutralizing antibodies in the plasma. In some aspects, if no anti-GLA neutralizing antibodies are present, the enzyme replacement therapy (e.g., recombinant α-Gal A enzyme) enters cells (e.g., endothelial cells) via the M6P receptor, leading to Gb3 clearance from lysosomes. In some aspects, if the anti-GLA neutralizing antibodies are present, they can neutralize the ERT activity by binding the enzyme (e.g., recombinant α-Gal A).

In some aspects, the anti-GLA neutralizing IgG antibody-tagged ERT molecules will be internalized and digested by macrophages. If more anti-GLA neutralizing antibodies than ERT are present, this can result in a decreased cellular Gb3 clearance. If the ERT dose exceeds the anti-GLA neutralizing antibody titers, more ERT can enter the lysosomes of target cells, resulting in increased Gb3 clearance. (Lenders et al., J Am Soc Nephrol 29:2265-2278 (2018).

The anti-GLA antibody neutralizing activity is described, for example, in Rombach et al., PLOS One 7: e47805 (2012); Lenders et al., J Am Soc Nephrol 27:256-264 (2016); Smid et al., Mol Genet Metab 108:132-137 (2013).

In some aspects, the anti-GLA neutralizing antibody is an IgG antibody. In some aspects, the anti-GLA neutralizing antibody is an IgG4 antibody. In some aspects, the anti-GLA neutralizing antibody is an IgG2 antibody. In some aspects, the anti-GLA neutralizing antibody is an IgG1 antibody.

In some aspects, the anti-GLA neutralizing antibodies can develop within about one month, about two months, about three months, about four months, about five months, about six months, about seven months, about eight months, about nine months, about ten months, about eleven months, or within about twelve months of starting the enzyme replacement therapy.

In some aspects, the human subject that can develop the anti-GLA neutralizing antibodies, for example, a male patient with classical Fabry disease, as described in e.g., Van der Veen et al., Mol Genet Metab. 126(2): 162-168 (2019); Wilcox et al., Mol Genet Metab. 105(3): 443-449 (2012).

In some aspects, the α-Gal A protein expressed from the transgene decreases the amount of glycosphingolipids in the subject by at least about 2 fold as compared to the amount of glycosphingolipids in the subject prior to the administration.

In some aspects, the α-Gal A protein expressed from the transgene decreases the amount of glycosphingolipids in the subject by about 10 percent (%), about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% as compared to the amount of glycosphingolipids in the subject prior to the administration.

In some aspects, the α-Gal A protein expressed from the transgene maintains the amount of glycosphingolipids in the subject at the same level as prior to the administration. In some aspects, the amounts of glycosphingolipids are maintained at the same level as prior to the administration in the subjects on a stable dose of an enzyme replacement therapy (ERT) pre-treatment as described herein. The term “stable dose of an enzyme replacement therapy” or “stable dose of the ERT” is defined as not having missed more than 3 doses of ERT during the 6 months prior to consent) and regimen (14 days #1 day for at least 3 months prior to enrollment). In some aspects, the subjects with low baseline levels of lyso-Gb3 (e.g., ERT treated subjects) can maintain the same level of lyso-Gb3 (e.g., lyso-Gb3 levels remain steady or show a modest, non-statistically significant decline) during observation period.

The term “ERT-naïve subject,” “ERT naïve subject,” or “an enzyme replacement therapy (ERT) naïve subject,” refers to the subject (e.g., human subject) who has never received the enzyme replacement therapy (ERT).

The term “ERT-pseudo-naïve subject,” “ERT pseudo-naïve subject,” or “ERT pseudo naïve subject,” refers to the subject (e.g., human subject) who has previously been on ERT but have not received ERT treatment in the past 6 months prior to the beginning of the clinical study as described herein.

In some aspects, glycosphingolipids comprise globotriaosylceramide (Gb3), globotriaosylsphingosine (lyso-Gb3), galabiosylceramide, or any combination thereof. In some aspects, Gb3 and/or lyso-Gb3 levels are measured in the subject's plasma and/or urine as described below in Example 2. In some aspects, Gb3 and/or lyso-Gb3 levels are measured in the subject's tissue.

In some aspects, the α-Gal A protein expressed from the transgene decreases the amount of glycosphingolipids in one or more of plasma, liver, heart, kidney, urine, skin, or spleen.

In some aspects, the α-Gal A protein activity in the subject is between about 0-fold higher to about 2-fold higher, between about 2-fold higher to about 5-fold higher, between about 5-fold higher to about 10-fold higher, between about 10-fold higher to about 20-fold higher, between about 20-fold higher to about 30-fold higher, between about 30-fold higher to about 40-fold higher, between about 30-fold higher to about 40-fold higher, between about 40-fold higher to about 50-fold higher, between about 50-fold higher to about 60-fold higher, between about 60-fold higher to about 70-fold higher, between about 70-fold higher to about 80-fold higher, between about 80-fold higher to about 90-fold higher, between about 90-fold higher to about 100-fold higher, between about 100-fold higher to about 200-fold higher, between about 200-fold higher to about 300-fold higher, between about 300-fold higher to about 400-fold higher, between about 400-fold higher to about 500-fold higher than the mean normal α-Gal A protein activity compared to the α-Gal A protein activity in the subject prior to the administration.

In some aspects, the α-Gal A protein activity in the subject is between about 0-fold higher to about 2-fold higher, between about 2-fold higher, about 3-fold higher, about 4-fold higher, about 5-fold higher, about 6-fold higher, about 7-fold higher, about 8-fold higher, about 9-fold higher, about 10-fold higher, about 11-fold higher, about 12-fold higher, about 13-fold higher, about 14-fold higher, about 15-fold higher, about 16-fold higher, about 17-fold higher, about 18-fold higher, about 19-fold higher, about 20-fold higher, about 21-fold higher, about 22-fold higher, about 23-fold higher, about 24-fold higher, about 25-fold higher, about 26-fold higher, about 27-fold higher, about 28-fold higher, about 29-fold higher, about 30-fold higher, about 31-fold higher, about 32-fold higher, about 33-fold higher, about 34-fold higher, about 35-fold higher, about 36-fold higher, about 37-fold higher, about 38-fold higher, about 39-fold higher, about 40-fold higher, about 41-fold higher, about 42-fold higher, about 43-fold higher, about 44-fold higher, about 45-fold higher, about 46-fold higher, about 47-fold higher, about 48-fold higher, about 49-fold higher, about 50-fold higher, about 51-fold higher, about 52-fold higher, about 53-fold higher, about 54-fold higher, about 55-fold higher, about 56-fold higher, about 57-fold higher, about 58-fold higher, about 59-fold higher, about 60-fold higher, about 61-fold higher, about 62-fold higher, about 63-fold higher, about 64-fold higher, about 65-fold higher, about 66-fold higher, about 67-fold higher, about 68-fold higher, about 69-fold higher, about 70-fold higher, about 71-fold higher, about 72-fold higher, about 73-fold higher, about 74-fold higher, about 75-fold higher, about 76-fold higher, about 77-fold higher, about 78-fold higher, about 79-fold higher, about 80-fold higher, about 81-fold higher, about 82-fold higher, about 83-fold higher, about 84-fold higher, about 85-fold higher, about 86-fold higher, about 87-fold higher, about 88-fold higher, about 89-fold higher, about 90-fold higher, about 91-fold higher, about 92-fold higher, about 93-fold higher, about 94-fold higher, about 95-fold higher, about 96-fold higher, about 97-fold higher, about 98-fold higher, about 99-fold higher, or about 100-fold higher than the mean normal α-Gal A protein activity compared to the α-Gal A protein activity in the subject prior to the administration.

In some aspects, the levels of the α-Gal A protein expressed from the transgene are measured in one or more of the subject's plasma, serum, whole blood, dried blood spot, leukocytes, or other blood components. In some aspects, the α-Gal A protein expressed from the transgene is active in the subject's kidneys, liver skin, and heart.

In some aspects, the expression of the at least one α-Gal A protein is sustained for at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

In some aspects, the subject is administered an immunosuppressant (e.g., prophylactic steroid treatment) prior to and/or during administration of the AAV expression vector. In some aspects, the immunosuppressant comprises prednisone.

In some aspects, the subject is not administered an immunosuppressant prior to and/or during administration of the AAV expression vector.

In some aspects, the subject is not administered a preconditioning treatment (e.g., a conditioning agent) prior to the administration of the AAV expression vector. The term “precondition” or “preconditioning,” as used herein refers to using a chemotherapy conditioning agent, such as for example, busulfan (Myleran®, GlaxoSmithKline, Busulfex®, Otsuka America Pharmaceutical, Inc.). The conditioning agent can be used, e.g., for ex vivo lentiviral gene therapy to deplete/kill the bone marrow cells in order to create space in the patient's bone marrow. Once the patient receives the gene therapy, the therapeutic stem cells are expected to engraft in the bone marrow and produce cells containing the therapeutic gene. The conditioning agent can have the adverse effects associated with chemotherapy (e.g., a severe effect in fertility).

In some aspects, the subject is not administered a conditioning agent or an immunosuppressant (e.g., prophylactic steroid treatment) prior to and/or during administration of the AAV expression vector.

In some aspects, the AAV expression vectors and pharmaceutical compositions of the present eliminate the need for biweekly enzyme replacement therapy (ERT) infusions.

In some aspects, the AAV expression vectors and pharmaceutical compositions of the present disclosure can preserve renal function in the subject with Fabry disease. In some aspects, an Estimated Glomerular filtration rate (eGFR) in ml/min/1.73m2 is measured in the subject after the administering using the Chronic Kidney Disease Epidemiology Collaboration (CKD-EPI) equation. (See e.g., Rombach S M et al., Nephrol Dial Transplant. 25(8):2549-2556 (2010)). In some aspects, the rate of annual eGFR decline is lower than in a comparable untreated subject with Fabry disease with Fabry disease.

In some aspects, the AAV expression vectors and pharmaceutical compositions of the present disclosure can reduce cardiac morbidity in the subject with Fabry disease. In some aspects, an Ejection Fraction (EF) is measured in the subject as stroke volume (SV)/left ventricular volumes at end-diastole (LVEDV) after the administering. (See e.g., Pieroni et al., Journal of the American College of Cardiology, 77(7): 922-936 (2021); Linhart A. The heart in Fabry disease. In: Mehta A, Beck M, Sunder-Plassmann G, editors. Fabry Disease: Perspectives from 5 Years of FOS. Oxford: Oxford PharmaGenesis; 2006. Chapter 20.) In some aspects, the rate of annual EF decline is lower than in a comparable untreated subject with Fabry disease.

In some aspects, a Global Longitudinal Strain (GLS) is measured in the subject by 2D strain echocardiography or cardiac magnetic resonance imaging (cardiac MRI or CMR) after the administering. (See e.g., Pieroni et al., Journal of the American College of Cardiology, 77(7): 922-936 (2021)). In some aspects, the annual shortening progression the contractibility of the muscles of the heart is lower than in a comparable untreated subject with Fabry disease.

In some aspects, a Left Ventricular Mass Index (LVMI) is measured as left ventricular mass (LVM)/body surface area in the subject after the administering. (See e.g., Pieroni et al., Journal of the American College of Cardiology, 77(7): 922-936 (2021)). In some aspects, the annual LVMI increase is lower than in a comparable untreated subject with Fabry disease.

In some aspects, the AAV expression vectors and pharmaceutical compositions of the present disclosure can improve one or more audiologic symptoms in the subject with Fabry disease. In some aspects, there is an improvement in one or more audiologic symptoms in the subject after the administration. In some aspects, one or more audiologic symptoms are tinnitus, vertigo, or progressive hearing loss.

In some aspects, the AAV expression vectors and pharmaceutical compositions of the present disclosure can improve perspiration in the subject with Fabry disease. In some aspects, the subject can have a positive change in the level of perspiration from anhidrosis to hypohidrosis or normal hidrosis.

### III. Adeno-Associated Virus (AAV) Expression Vectors

AAV, a parvovirus belonging to the genus Dependovirus, has several attractive features not found in other viruses. For example, AAV can infect a wide range of host cells, including non-dividing cells. Furthermore, AAV can infect cells from different species. Importantly, AAV has not been associated with any human or animal disease, and does not appear to alter the physiological properties of the host cell upon integration. Finally, AAV is stable at a wide range of physical and chemical conditions, which lends itself to production, storage, and transportation requirements.

The AAV genome, a linear, single-stranded DNA molecule containing approximately 4700 nucleotides (the AAV-2 genome consists of 4681 nucleotides), generally comprises an internal non-repeating segment flanked on each end by inverted terminal repeats (ITRs). The ITRs are approximately 145 nucleotides in length (AAV-1 has ITRs of 143 nucleotides) and have multiple functions, including serving as origins of replication, and as packaging signals for the viral genome.

The internal non-repeated portion of the genome includes two large open reading frames (ORFs), known as the AAV replication (rep) and capsid (cap) regions. These ORFs encode replication and capsid gene products, respectively: replication and capsid gene products (i.e., proteins) allow for the replication, assembly, and packaging of a complete AAV virion. More specifically, a family of at least four viral proteins are expressed from the AAV rep region: Rep 78, Rep 68, Rep 52, and Rep 40, all of which are named for their apparent molecular weights. The AAV cap region encodes at least three proteins: VP1, VP2, and VP3.

AAV is a helper-dependent virus, requiring co-infection with a helper virus (e.g., adenovirus, herpesvirus, or vaccinia virus) in order to form functionally complete AAV virions. In the absence of co-infection with a helper virus, AAV establishes a latent state in which the viral genome inserts into a host cell chromosome or exists in an episomal form, but infectious virions are not produced. Subsequent infection by a helper virus “rescues” the integrated genome, allowing it to be replicated and packaged into viral capsids, thereby reconstituting the infectious virion. While AAV can infect cells from different species, the helper virus must be of the same species as the host cell. Thus, for example, human AAV will replicate in canine cells that have been co-infected with a canine adenovirus.

To produce recombinant AAV (rAAV) virions containing the HNA, a suitable host cell line is transfected with an AAV vector containing the HNA, but lacking rep and cap. The host cell is then infected with wild-type (wt) AAV and a suitable helper virus to form rAAV virions. Alternatively, wt AAV genes (known as helper function genes, comprising rep and cap) and helper virus function genes (known as accessory function genes) can be provided in one or more plasmids, thereby eliminating the need for wt AAV and helper virus in the production of rAAV virions. The helper and accessory function gene products are expressed in the host cell where they act in trans on the rAAV vector containing the heterologous gene. The heterologous gene is then replicated and packaged as though it were a wt AAV genome, forming a recombinant AAV virion. When a patient's cells are transduced with the resulting rAAV virion, the HNA enters and is expressed in the patient's cells. Because the patient's cells lack the rep and cap genes, as well as the accessory function genes, the rAAV virion cannot further replicate and package its genomes. Moreover, without a source of rep and cap genes, wt AAV virions cannot be formed in the patient's cells. See e.g., U.S. Appl. Publ. No. 2003/0147853.

In some aspects, AAV expression vectors of the present disclosure can comprise or be derived from any natural or recombinant AAV serotype. According to the present disclosure, the AAV serotype can be, but is not limited to AAV1, AAV2, AAV3, AAV4, AAV5, AAV6, AAV7, AAV8, AAVrh8, AAV9, AAV10, AAVrh10, AAV11, AAV12, and AAV2/6. In some aspects, the AAV serotype is AAV2/6. In some aspects, the AAV serotype is AAV2. In some aspects, the AAV serotype is AAV6.

The AAV expression vectors of the present disclosure can comprise the expression cassette, which can comprise any promoter, enhancer, intron, signal peptide, GLA-coding, poly A sequence, or Woodchuck Hepatitis Virus (WHV) Posttranscriptional Regulatory Element (WPRE) sequence. In some aspects, the enhancer and/or promoter is liver-specific, for example, comprised of a human apolipoprotein E (APOE) enhancer and a human alpha 1-antitrypsin (hAAT) promoter (Miao C H et al., Mol. Ther. 1(6): 522-532 (2000)). In some aspects, the liver specific promoter comprises one or more ApoE enhancer sequences (e.g., 1, 2, 3 and/or 4; see Okuyama et al., Hum Gen Ther 7(5):637-645 (1996)). In some aspects, the promoter is linked to an intron. In some aspects, the intron is a human hemoglobin beta (HBB)-IGG chimeric intron comprising the 5′ donor site from the first intron of the human β-globin gene and the branch and 3′ acceptor site from the intron of an immunoglobulin gene heavy chain variable region. In some aspects, the ApoE/hAAT promoter is specifically and highly active in hepatocytes, the intended target tissue, but is inactive in non-liver cell and tissue types; which reduces or prevents expression and activity in non-target tissues. In some aspects, the signal peptide comprises an α-GalA signal peptide (e.g., a human α-GalA signal peptide) and the polyadenylation signal comprises a bovine growth hormone (bGH) poly A signal sequence. The WPRE sequence can be any wild-type or mutated WPRE sequence. See, e.g., U.S. Pat. No. 10,179,918. In some aspects, the WPRE sequence comprises a mutated WPRE such as the mut6 WPRE sequence.

In some aspects, the AAV expression vector of the present disclosure comprises the α-Gal A expression cassette flanked by two ITRs. These two ITRs are at the 5′ and 3′ ends of the α-Gal A expression cassette.

In some aspects, the AAV expression vectors of the present disclosure comprise the α-Gal A expression cassette, which comprises an α galactosidase A (α-Gal A) transgene encoding the at least one α-Gal A protein. In some aspects, the α-Gal A transgene comprises a wild-type sequence of the functioning α-Gal A gene. In some aspects, the AAV expression vectors of the present disclosure comprise the corrective α-Gal A transgene. The sequence of the corrective α-Gal A transgene is altered in some manner to give enhanced biological activity (e.g., optimized codons to increase biological activity and/or alteration of transcriptional and translational regulatory sequences to improve gene expression). In some aspects, the α-Gal A gene is modified to improve expression characteristics. Such modifications can include, but are not limited to, insertion of a translation start site (e.g. methionine), addition of an optimized Kozak sequence, insertion of a signal peptide, and/or codon optimization.

In some aspects, the AAV expression vector as described herein is AAV-001, which is also referred to as Variant #21 in International Publication No. WO/2020/142752 (which is incorporated herein by reference in its entirety).

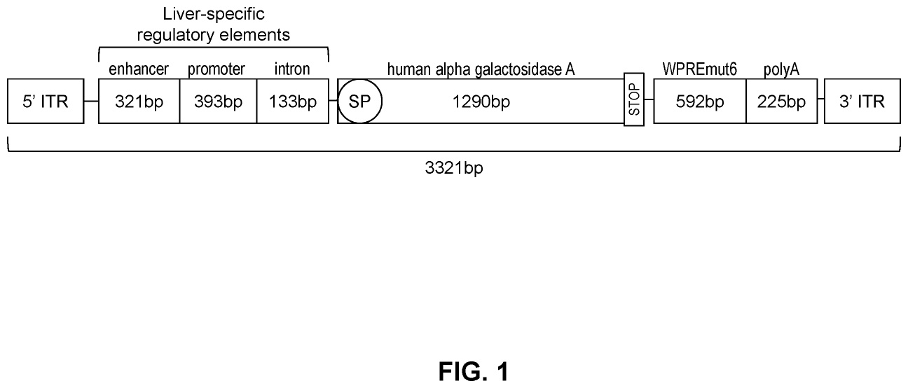

The rAAV vector of the present disclosure (e.g., AAV-001 rAAV vector) comprises human α-Gal A (hGLA) expression cassette (3321 bp) that includes liver-specific regulatory elements that drive expression of a hGLA transgene (see FIG. 1). The hGLA transgene is under the control of an enhancer and hepatic control region from the human apolipoprotein E (ApoE) gene and the human α-1-antitrypsin (hAAT) promoter. A modified chimeric intron (HBB-IghGLA transgene comprises a codon-optimized hGLA α-Gal A enzyme that has the same amino acid sequence as the native hGLA protein and an approved recombinant α-Gal A (Fabrazyme®).

The α-Gal A expression cassette of the present disclosure contains a mutated form of the woodchuck hepatitis virus (WHV) posttranscriptional regulatory element (WPREmut6). WPREmut6 is a 592-bp DNA sequence containing the promoter region of WHV X protein followed by a truncated form of the X protein itself (WPRE, Zufferey et al., J Virol. 73(4): 2886-2892 (1999)) with point mutations in the putative promoter region and start codon of the X protein open reading frame to prevent X protein expression (mut6, Zanta-Boussif et al., Gene Therapy 16(5): 605-619 (2009)). The poly A sequence is a derivative of the bovine growth hormone polyadenylation signal. The addition of the WPREmut6 element led to increased α-Gal A protein production. Indeed, greater potency was noted with AAV-001 compared to AAV-001PC (that lacks the WPREmut6 element). The AAV-001 rAAV vector comprises the α-Gal A expression cassette flanked on each end by ITRs derived from AAV2, wherein the α-Gal A expression cassette is packaged with capsid derived from adeno-associated virus type 6 (AAV6) using a Sf9 insect cell/recombinant baculovirus (Sf9/rBV) expression system.

In some aspects, the AAV expression vector (e.g., AAV-001 rAAV vector) sequence comprises the elements and sequence of the α-Gal A expression cassette, as shown below in Table 1.

TABLE 1
α-Gal A cDNA elements and complete sequence

| | | SEQ | |
|---|---|---|---|
| | | ID | |
| Element | Location | NO | Sequence |
| 5′ ITR | 1-130 | 1 | ctgcgcgctc gctcgctcac tgaggccgcc cgggcaaagc ccgggcgtcg ggcgaccttt |
| | | | ggtcgcccgg cctcagtgag cgagcgagcg cgcagagagg gagtggccaa ctccatcact |
| | | | aggggttcct |
| ApoE | 141-461 | 2 | AGGCTCAGAGGCACACAGGAGTTTCTGGGCTCACCCTGCCC |
| enhancer | | | CCTTCCAACCCCTCAGTTCCCATCCTCCAGCAGCTGTTTGTG |
| | | | TGCTGCCTCTGAAGTCCACACTGAACAAACTTCAGCCTACT |
| | | | CATGTCCCTAAAATGGGCAAACATTGCAAGCAGCAAACAG |
| | | | CAAACACACAGCCCTCCCTGCCTGCTGACCTTGGAGCTGGG |
| | | | GCAGAGGTCAGAGACCTCTCTGGGCCCATGCCACCTCCAAC |
| | | | ATCCACTCGACCCCTTGGAATTTCGGTGGAGAGGAGCAGA |
| | | | GGTTGTCCTGGCGTGGTTTAGGTAGTGTGAGAGGG |
| hAAT | 471-863 | 3 | gatcttgcta ccagtggaac agccactaag gattctgcag tgagagcaga gggccagcta |
| promoter | | | agtggtactc tcccagagac tgtctgactc acgccacccc ctccaccttg gacacaggac |
| | | | gctgtggttt ctgagccagg tacaatgact cctttcggta agtgcagtgg aagctgtaca |
| | | | ctgcccaggc aaagcgtccg ggcagcgtag gcgggcgact cagatcccag ccagtggact |
| | | | tagcccctgt ttgctcctcc gataactggg gtgaccttgg ttaatattca ccagcagcct |
| | | | cccccgttgc ccctctggat ccactgctta aatacggacg aggacagggc cctgtctcct |
| | | | cagcttcagg caccaccact gacctgggac agt |
| HBB-IgG | 867-999 | 4 | gtaagtatca aggttacaag acaggtttaa ggagaccaat agaaactggg cttgtcgaga |
| chimeric | | | cagagaagac tcttgcgttt ctgataggca cctattggtc ttactgacat ccactttgcc |
| intron | | | tttctctcca cag |
| GLA | 1052-2341 | 5 | ATGCAACTTAGGAACCCCGAACTTCATCTTGGC |
| CDNA | | | TGCGCCCTGGCCCTCCGCTTCCTCGCTCTCGTTTCTTG |
| | | | GGACATCCCTGGCGCTAGGGCACTCGACAACGGCCTC |
| | | | GCGCGGACTCCTACGATGGGATGGTTGCACTGGGAAA |
| | | | GGTTTATGTGCAATCTGGATTGCCAGGAGGAGCCGGA |
| | | | CTCATGCATCTCGGAGAAGCTGTTCATGGAGATGGCG |
| | | | GAACTTATGGTATCGGAGGGATGGAAGGATGCCGGGT |
| | | | ATGAGTATCTCTGTATCGACGATTGTTGGATGGCTCCC |
| | | | CAGAGAGACTCCGAGGGACGACTCCAAGCGGACCCC |
| | | | CAGCGCTTTCCACATGGCATTCGACAGCTCGCCAATTA |
| | | | CGTGCACTCGAAGGGGTTGAAGTTGGGAATCTACGCA |
| | | | GATGTGGGCAACAAAACGTGTGCGGGGTTCCCGGGGT |
| | | | CGTTTGGATACTACGATATTGATGCGCAGACGTTTGCT |
| | | | GACTGGGGTGTCGATCTTTTGAAATTTGATGGCTGTTA |
| | | | CTGTGATTCGTTGGAAAACCTGGCGGATGGATACAAG |
| | | | CATATGTCACTCGCCTTGAACCGGACAGGTCGCTCAA |
| | | | TCGTATACAGCTGCGAATGGCCCCTCTATATGTGGCCC |
| | | | TTCCAAAAGCCCAATTACACAGAGATTCGGCAGTATT |
| | | | GCAATCACTGGAGGAACTTTGCCGATATTGACGACAG |
| | | | CTGGAAATCCATCAAGTCCATTCTCGATTGGACGAGC |
| | | | TTCAACCAGGAGCGCATCGTGGACGTGGCAGGACCCG |
| | | | GAGGTTGGAACGATCCGGACATGCTCGTAATTGGGAA |
| | | | TTTCGGGCTTAGCTGGAATCAGCAAGTCACCCAAATG |
| | | | GCGCTGTGGGCCATCATGGCAGCTCCTCTCTTTATGTC |
| | | | GAATGATCTGCGGCATATCTCGCCCCAGGCAAAGGCT |
| | | | CTTTTGCAAGACAAGGACGTCATCGCAATCAATCAGG |
| | | | ACCCATTGGGGAAACAGGGATATCAACTTCGCCAGGG |
| | | | TGACAATTTCGAAGTATGGGAGAGGCCGCTTAGCGGG |
| | | | CTGGCGTGGGCGGTCGCGATGATTAACCGGCAGGAAA |
| | | | TCGGAGGGCCTCGCTCGTATACCATCGCAGTGGCCTC |
| | | | ACTGGGCAAAGGAGTGGCGTGCAATCCGGCCTGCTTC |
| | | | ATCACCCAGTTGTTGCCCGTCAAAAGAAAGCTGGGTT |
| | | | TCTACGAGTGGACATCCAGACTTAGATCACACATTAA |
| | | | CCCTACTGGTACGGTGTTGCTCCAGCTCGAAAACACA |
| | | | ATGCAGATGTCGTTGAAAGACCTGCTGTAA |
| WPRE | 2364-2955 | 6 | aatcaacctc tggattacaa aatttgtgaa agattgactg gtattcttaa ctatgttgct |
| mut6 | | | ccttttacgc tatgtggata cgctgcttta atgcctttgt atcatgctat tgcttcccgt |
| J04515 | | | atggctttca ttttctcctc cttgtataaa tcctggttgc tgtctcttta tgaggagttg |
| | | | tggcccgttg tcaggcaacg tggcgtggtg tgcactgtgt ttgctgacgc aacccccact |
| | | | ggttggggca ttgccaccac ctgtcagctc ctttccggga ctttcgcttt ccccctccct |
| | | | attgccacgg cggaactcat cgccgcctgc cttgcccgct gctggacagg ggctcggctg |
| | | | ttgggcactg acaattccgt ggtgttgtcg gggaaatcat cgtcctttcc ttggctgctc |
| | | | gcctgtgttg ccacctggat tctgcgcggg acgtccttct gctacgtccc ttcggccctc |
| | | | aatccagcgg accttccttc ccgcggcctg ctgccggctc tgcggcctct tccgcgtctt |
| | | | cgccttcgcc ctcagacgag tcggatctcc ctttgggccg cctccccgcc tg |
| bGH | 2962-3186 | 7 | ctgtgccttc tagttgccag ccatctgttg tttgcccctc ccccgtgcct tccttgaccc |
| polyA | | | tggaaggtgc cactcccact gtcctttcct aataaaatga ggaaattgca tcgcattgtc |
| | | | tgagtaggtg tcattctatt ctggggggtg gggtggggca ggacagcaag ggggaggatt |
| | | | gggaagacaa tagcaggcat gctggggatg cggtgggctc tatgg |
| 3′ ITR | 3214-3321 | 8 | aggaacccct agtgatggag ttggccactc cctctctgcg cgctcgctcg ctcactgagg |
| | | | ccgcccgggc tttgcccggg cggcctcagt gagcgagcga gcgcgcag |

SEQ ID NO: 9 (complete transgene equence)

| | |
|---|---|
| CTGCGCGCTC GCTCGCTCAC TGAGGCCGCC CGGGCAAAGC CCGGGCGTCG | 50 |
| GGCGACCTTT GGTCGCCCGG CCTCAGTGAG CGAGCGAGCG CGCAGAGAGG | 100 |
| GAGTGGCCAA CTCCATCACT AGGGGTTCCT GCGGCCTAGT AGGCTCAGAG | 150 |
| GCACACAGGA GTTTCTGGGC TCACCCTGCC CCCTTCCAAC CCCTCAGTTC | 200 |
| CCATCCTCCA GCAGCTGTTT GTGTGCTGCC TCTGAAGTCC ACACTGAACA | 250 |
| AACTTCAGCC TACTCATGTC CCTAAAATGG GCAAACATTG CAAGCAGCAA | 300 |
| ACAGCAAACA CACAGCCCTC CCTGCCTGCT GACCTTGGAG CTGGGGCAGA | 350 |
| GGTCAGAGAC CTCTCTGGGC CCATGCCACC TCCAACATCC ACTCGACCCC | 400 |
| TTGGAATTTC GGTGGAGAGG AGCAGAGGTT GTCCTGGCGT GGTTTAGGTA | 450 |
| GTGTGAGAGG GGTACCCGGG GATCTTGCTA CCAGTGGAAC AGCCACTAAG | 500 |
| GATTCTGCAG TGAGAGCAGA GGGCCAGCTA AGTGGTACTC TCCCAGAGAC | 550 |
| TGTCTGACTC ACGCCACCCC CTCCACCTTG GACACAGGAC GCTGTGGTTT | 600 |
| CTGAGCCAGG TACAATGACT CCTTTCGGTA AGTGCAGTGG AAGCTGTACA | 650 |
| CTGCCCAGGC AAAGCGTCCG GGCAGCGTAG GCGGGCGACT CAGATCCCAG | 700 |
| CCAGTGGACT TAGCCCCTGT TTGCTCCTCC GATAACTGGG GTGACCTTGG | 750 |
| TTAATATTCA CCAGCAGCCT CCCCCGTTGC CCCTCTGGAT CCACTGCTTA | 800 |
| AATACGGACG AGGACAGGGC CCTGTCTCCT CAGCTTCAGG CACCACCACT | 850 |
| GACCTGGGAC AGTCAGGTAA GTATCAAGGT TACAAGACAG GTTTAAGGAG | 900 |
| ACCAATAGAA ACTGGGCTTG TCGAGACAGA GAAGACTCTT GCGTTTCTGA | 950 |
| TAGGCACCTA TTGGTCTTAC TGACATCCAC TTTGCCTTTC TCTCCACAGG | 1000 |
| CAATTGATCC CCCTGATCTG CGGCCTCGAC GGTATCGATA AGCTTGCCAC | 1050 |
| CATGCAACTT AGGAACCCCG AACTTCATCT TGGCTGCGCC CTGGCCCTCC | 1100 |
| GCTTCCTCGC TCTCGTTTCT TGGGACATCC CTGGCGCTAG GGCACTCGAC | 1150 |
| AACGGCCTCG CGCGGACTCC TACGATGGGA TGGTTGCACT GGGAAAGGTT | 1200 |
| TATGTGCAAT CTGGATTGCC AGGAGGAGCC GGACTCATGC ATCTCGGAGA | 1250 |
| AGCTGTTCAT GGAGATGGCG GAACTTATGG TATCGGAGGG ATGGAAGGAT | 1300 |
| GCCGGGTATG AGTATCTCTG TATCGACGAT TGTTGGATGG CTCCCCAGAG | 1350 |
| AGACTCCGAG GGACGACTCC AAGCGGACCC CCAGCGCTTT CCACATGGCA | 1400 |
| TTCGACAGCT CGCCAATTAC GTGCACTCGA AGGGGTTGAA GTTGGGAATC | 1450 |
| TACGCAGATG TGGGCAACAA AACGTGTGCG GGGTTCCCGG GGTCGTTTGG | 1500 |
| ATACTACGAT ATTGATGCGC AGACGTTTGC TGACTGGGGT GTCGATCTTT | 1550 |
| TGAAATTTGA TGGCTGTTAC TGTGATTCGT TGGAAAACCT GGCGGATGGA | 1600 |
| TACAAGCATA TGTCACTCGC CTTGAACCGG ACAGGTCGCT CAATCGTATA | 1650 |
| CAGCTGCGAA TGGCCCCTCT ATATGTGGCC CTTCCAAAAG CCCAATTACA | 1700 |
| CAGAGATTCG GCAGTATTGC AATCACTGGA GGAACTTTGC CGATATTGAC | 1750 |
| GACAGCTGGA AATCCATCAA GTCCATTCTC GATTGGACGA GCTTCAACCA | 1800 |
| GGAGCGCATC GTGGACGTGG CAGGACCCGG AGGTTGGAAC GATCCGGACA | 1850 |
| TGCTCGTAAT TGGGAATTTC GGGCTTAGCT GGAATCAGCA AGTCACCCAA | 1900 |
| ATGGCGCTGT GGGCCATCAT GGCAGCTCCT CTCTTTATGT CGAATGATCT | 1950 |
| GCGGCATATC TCGCCCCAGG CAAAGGCTCT TTTGCAAGAC AAGGACGTCA | 2000 |
| TCGCAATCAA TCAGGACCCA TTGGGGAAAC AGGGATATCA ACTTCGCCAG | 2050 |
| GGTGACAATT TCGAAGTATG GGAGAGGCCG CTTAGCGGGC TGGCGTGGGC | 2100 |
| GGTCGCGATG ATTAACCGGC AGGAAATCGG AGGGCCTCGC TCGTATACCA | 2150 |
| TCGCAGTGGC CTCACTGGGC AAAGGAGTGG CGTGCAATCC GGCCTGCTTC | 2200 |
| ATCACCCAGT TGTTGCCCGT CAAAAGAAAG CTGGGTTTCT ACGAGTGGAC | 2250 |
| ATCCAGACTT AGATCACACA TTAACCCTAC TGGTACGGTG TTGCTCCAGC | 2300 |
| TCGAAAACAC AATGCAGATG TCGTTGAAAG ACCTGCTGTA ATCTAGAGGA | 2350 |
| TCTCGAGAGA TCTAATCAAC CTCTGGATTA CAAAATTTGT GAAAGATTGA | 2400 |
| CTGGTATTCT TAACTATGTT GCTCCTTTTA CGCTATGTGG ATACGCTGCT | 2450 |
| TTAATGCCTT TGTATCATGC TATTGCTTCC CGTATGGCTT TCATTTTCTC | 2500 |
| CTCCTTGTAT AAATCCTGGT TGCTGTCTCT TTATGAGGAG TTGTGGCCCG | 2550 |
| TTGTCAGGCA ACGTGGCGTG GTGTGCACTG TGTTTGCTGA CGCAACCCCC | 2600 |
| ACTGGTTGGG GCATTGCCAC CACCTGTCAG CTCCTTTCCG GGACTTTCGC | 2650 |
| TTTCCCCCTC CCTATTGCCA CGGCGGAACT CATCGCCGCC TGCCTTGCCC | 2700 |
| GCTGCTGGAC AGGGGCTCGG CTGTTGGGCA CTGACAATTC CGTGGTGTTG | 2750 |
| TCGGGGAAAT CATCGTCCTT TCCTTGGCTG CTCGCCTGTG TTGCCACCTG | 2800 |
| GATTCTGCGC GGGACGTCCT TCTGCTACGT CCCTTCGGCC CTCAATCCAG | 2850 |
| CGGACCTTCC TTCCCGCGGC CTGCTGCCGG CTCTGCGGCC TCTTCCGCGT | 2900 |
| CTTCGCCTTC GCCCTCAGAC GAGTCGGATC TCCCTTTGGG CCGCCTCCCC | 2950 |
| GCCTGGGATC TCTGTGCCTT CTAGTTGCCA GCCATCTGTT GTTTGCCCCT | 3000 |
| CCCCCGTGCC TTCCTTGACC CTGGAAGGTG CCACTCCCAC TGTCCTTTCC | 3050 |
| TAATAAAATG AGGAAATTGC ATCGCATTGT CTGAGTAGGT GTCATTCTAT | 4000 |
| TCTGGGGGGT GGGGTGGGGC AGGACAGCAA GGGGGAGGAT TGGGAAGACA | 4050 |
| ATAGCAGGCA TGCTGGGGAT GCGGTGGGCT CTATGGACCG GTCTCGAGAT | 4100 |
| CCACTAGGGC CGCAGGAACC CCTAGTGATG GAGTTGGCCA CTCCCTCTCT | 4150 |
| GCGCGCTCGC TCGCTCACTG AGGCCGCCCG GGCTTTGCCC GGGCGGCCTC | 4200 |
| AGTGAGCGAG CGAGCGCGCA G (SEQ ID NO: 9) | 4221 |

In some aspects, the AAV expression vector of the present disclosure (e.g., AAV-001 rAAV vector) comprises an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence. In some aspects, the enhancer comprises the nucleotide sequence as set forth in SEQ ID NO: 2, the promotor comprises the nucleotide sequence as set forth in SEQ ID NO: 3, the intron comprises the nucleotide sequence as set forth in SEQ ID NO: 4, the α-GalA transgene comprises the nucleotide sequence as set forth in SEQ ID NO: 5, the mutated WPRE sequence comprises the nucleotide sequence as set forth in SEQ ID NO: 6, and the poly A signal sequence comprises the nucleotide sequence as set forth in SEQ ID NO: 7.

In some aspects, the α-Gal A expression cassette of the present disclosure comprises the nucleotide sequence as set forth in SEQ ID NO: 9.

In some aspects, signal peptide of human α-galactosidase A comprises the amino acid sequence as set forth in SEQ ID NO: 10.

Human α-galactosidase A signal peptide
(SEQ ID NO: 10)
MQLRNPELHLGCALALRFLALVSWDIPGARA

### IV. Pharmaceutical Compositions and Formulations

In some aspects, the AAV expression vectors useful in the methods and compositions disclosed herein are present in a pharmaceutical composition. As such, some aspects of the present disclosure are directed to a pharmaceutical composition comprising an AAV expression vector of the present disclosure with a pharmaceutically acceptable carrier, diluent, solubilizer, emulsifier, preservative and/or adjuvant.

In some aspects, the pharmaceutically acceptable carrier comprises phosphate buffered saline containing CaCl₂), MgCl₂, NaCl, Sucrose, and Kolliphor (Poloxamer) P 188.

In some aspects, acceptable formulation materials preferably are nontoxic to recipients at the dosages and concentrations employed. In some aspects, the formulation material(s) are for s.c. and/or I.V. administration. In some aspects, the pharmaceutical composition comprises formulation materials for modifying, maintaining or preserving, for example, the pH, osmolality, viscosity, clarity, color, isotonicity, odor, sterility, stability, rate of dissolution or release, adsorption or penetration of the composition. In some aspects, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company (1995).

In some aspects, the optimal pharmaceutical composition will be determined by one skilled in the art depending upon, for example, the intended route of administration, delivery format and desired dosage. See, for example, Remington's Pharmaceutical Sciences, supra. In some aspects, such compositions influence the physical state, stability, rate of in vivo release and/or rate of in vivo clearance of the AAV expression vector.

In some aspects, the primary vehicle or carrier in a pharmaceutical composition is either aqueous or non-aqueous in nature. For example, in some aspects, a suitable vehicle or carrier is water for injection, physiological saline solution or artificial cerebrospinal fluid, possibly supplemented with other materials common in compositions for parenteral administration. In some aspects, the saline comprises isotonic phosphate-buffered saline. In some aspects, neutral buffered saline or saline mixed with serum albumin are further exemplary vehicles. In some aspects, pharmaceutical compositions comprise Tris buffer of about pH 7.0-8.5, or acetate buffer of about pH 4.0-5.5. In some aspects, the pharmaceutical composition further comprises sorbitol or a suitable substitute therefore. In some aspects, a composition comprising an AAV expression vector is prepared for storage by mixing the selected composition having the desired degree of purity with optional formulation agents (Remington's Pharmaceutical Sciences, supra) in the form of a lyophilized cake or an aqueous solution. Further, in some aspects, a composition comprising an AAV expression vector formulated as a lyophilizate using appropriate excipients such as sucrose.

In some aspects, the pharmaceutical composition is selected for parenteral delivery.

In some aspects, the formulation components are present in concentrations that are acceptable to the site of administration. In some aspects, buffers are used to maintain the composition at physiological pH or at a slightly lower pH, typically within a pH range of from about 5 to about 8.

In some aspects, when parenteral administration is contemplated, a therapeutic composition is in the form of a pyrogen-free, parenterally acceptable aqueous solution comprising an AAV expression vector of the present disclosure, in a pharmaceutically acceptable vehicle. In some aspects, a vehicle for parenteral injection is sterile distilled water in which an AAV expression vector is formulated as a sterile, isotonic solution, and properly preserved. In some aspects, the preparation involves the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In some aspects, hyaluronic acid is also used. Hyaluronic acid, when present, can have the effect of promoting sustained duration in the circulation. In some aspects, implantable drug delivery devices are used to introduce the desired molecule.

In some aspects, a pharmaceutical composition involves an effective quantity of an AAV expression vector in a mixture with non-toxic excipients which are suitable for the manufacture of tablets. In some aspects, by dissolving the tablets in sterile water, or another appropriate vehicle, solutions are prepared in unit-dose form. In some aspects, suitable excipients include, but are not limited to, inert diluents, such as calcium carbonate, sodium carbonate or bicarbonate, lactose, or calcium phosphate; or binding agents, such as starch, gelatin, or acacia; or lubricating agents such as magnesium stearate, stearic acid, or talc.

Additional pharmaceutical compositions will be evident to those skilled in the art, including formulations involving an AAV expression vector in sustained- or controlled-delivery formulations. In some aspects, techniques for formulating a variety of other sustained- or controlled-delivery means, such as liposome carriers, bio-erodible microparticles or porous beads and depot injections, are also known to those skilled in the art. See for example, PCT Application No. PCT/US93/00829 which describes the controlled release of porous polymeric microparticles for the delivery of pharmaceutical compositions. In some aspects, sustained-release preparations can include semipermeable polymer matrices in the form of shaped articles, e.g. films, or microcapsules. Sustained release matrices can include polyesters, hydrogels, polylactides (U.S. Pat. No. 3,773,919 and EP 058,481), copolymers of L-glutamic acid and gamma ethyl-L-glutamate (Sidman et al., Biopolymers, 22:547-556 (1983)), poly (2-hydroxyethyl-methacrylate) (Langer et al., J. Biomed. Mater. Res., 15:167-277 (1981) and Langer, Chem. Tech., 12:98-105 (1982)), ethylene vinyl acetate (Langer et al., supra) or poly-D(−)-3-hydroxybutyric acid (EP 133,988). In some aspects, sustained release compositions can also include liposomes, which can be prepared by any of several methods known in the art. See, e.g., Eppstein et al, Proc. Natl. Acad. Sci. USA, 82:3688-3692 (1985); EP 036,676; EP 088,046 and EP 143,949.

The pharmaceutical composition to be used for in vivo administration typically is sterile. In some aspects, this is accomplished by filtration through sterile filtration membranes. In some aspects, where the composition is lyophilized, sterilization using this method is conducted either prior to or following lyophilization and reconstitution. In some aspects, the composition for parenteral administration is stored in lyophilized form or in a solution. In some aspects, parenteral compositions generally are placed into a container having a sterile access port, for example, an intravenous solution bag or vial having a stopper pierceable by a hypodermic injection needle.

In some aspects, once the pharmaceutical composition has been formulated, it is stored in sterile vials as a solution, suspension, gel, emulsion, solid, or as a dehydrated or lyophilized powder. In some aspects, such formulations are stored either in a ready-to-use form or in a form (e.g., lyophilized) that is reconstituted prior to administration.

### V. Fabry Disease Therapies

In some aspects, the disclosure is directed to methods of treating Fabry disease in a human subject with the AAV expression vectors of the present disclosure, wherein the subject has been administered with an enzyme replacement therapy (ERT) for Fabry disease prior to the administering (“pre-treatment”) or a non-enzyme replacement therapy for Fabry disease for Fabry disease prior to the administering (“pre-treatment”).

### V.A Enzyme Replacement Therapy

In some aspects, the pre-treatment ERT comprises an enzyme replacement therapy.

In some aspects, the pre-treatment ERT comprises a recombinant α Galactosidase A (GLA) protein or a gene expressing GAL. In some aspects, the pre-treatment ERT comprises agalsidase alpha and/or beta or a gene expressing agalsidase alpha and/or beta. In some aspects, the enzyme replacement therapy comprises administering agalsidase alfa (Replagal®, Shire Human Genetic Therapies), agalsidase beta (Fabrazyme®; Sanofi Genzyme), pegunigalsidase alfa (PRX-102; Protalix BioTherapeutics), or any combination thereof. These forms of ERT are intended to compensate for a patient's inadequate α-Gal A activity with a recombinant form of the enzyme, administered intravenously. ERT has been demonstrated to reduce Gb3 deposition in capillary endothelium of the kidney and some other cell types. While ERT is effective in many settings, the treatment also has limitations. ERT has not been demonstrated to decrease the risk of stroke, cardiac muscle responds slowly, and Gb3 elimination from some of the cell types of the kidneys is limited. Some patients develop immune reactions to ERT. See e.g., U.S. Pat. No. 10,155,027.

In some aspects, about 0.2 mg/kg body weight of agalsidase alfa is infused every 2 weeks as an intravenous infusion.

In some aspects, about 0.3 mg/kg body weight of agalsidase beta is infused every 2 weeks as an intravenous infusion. In some aspects, about 1 mg/kg body weight of agalsidase beta is infused every 2 weeks as an intravenous infusion.

In some aspects, saturating the higher anti-GLA neutralizing antibody levels in a subject via higher ERT dosing can provide clinical benefit (e.g., decrease plasma lyso-Gb3 levels). See e.g., Lenders et al., Orphanet Journal of Rare Diseases, 13(171) (2018).

In some aspects, the pre-treatment ERT comprises a gene therapy. In some aspects, the gene therapy comprises a vector encoding the enzyme. In some aspects, the vector is a viral vector. In some aspects, the viral vector comprises an adeno-associated virus (AAV) vector or a lentiviral vector. In some aspects, the gene therapy comprises administering AVR-RD-01 (AvroBio), FLT-190 (Freeline Therapeutics), pegunigalsidase alfa (PRX-102; Protalix BioTherapeutics), and 4D-310 (4D Molecular Therapeutics), or any combination thereof.

AVR-RD-01 drug product comprises autologous CD34+ cell-enriched fraction that contains cells transduced with Lentiviral Vector/alpha-galactosidase A (AGA) encoding for the human AGA complementary deoxyribonucleic acid (cDNA) sequence. (ClinicalTrials.gov; Identifier: NCT03454893).

FLT190 is a single stranded (ss) AAV gene therapy construct with a codon-optimized human GLA cDNA driven by a liver specific promotor (FRE1), pseudotyped with AAV8 capsid (ssAAV8-FRE1-GLAco). (Nephron Clinical Practice, Abstracts: 6th Update on Fabry Disease: Biomarkers, Progression and Treatment Opportunities, May 26-28, 2019, Prague, Czech Republic).

Pegunigalsidase alfa (PRX-102) is an investigational, plant cell culture-expressed, and chemically modified stabilized version of the recombinant α-Galactosidase-A enzyme. Protein sub-units are covalently bound via chemical cross-linking using short PEG moieties, resulting in a molecule with unique pharmacokinetic parameters. In clinical studies, PRX-102 has been observed to have a circulatory half-life of approximately 80 hours.

4D-310 an adeno-associated virus (AAV) gene therapy comprised of two active components: the capsid (4D-C102) and the transgene cassette, which encodes a codon-optimized full length human GLA transgene driven by the CAG promoter. 4D-310 has been engineered so that it cannot replicate (replication incompetent). (ClinicalTrials.gov; Identifier: NCT04519749).

In some aspects, the gene therapy comprises a vector encoding the enzyme. In some aspects, the vector comprises an mRNA encoding a human GLA protein or agalsidase alpha and/or beta, as described in e.g., U.S. Pat. No. 9,308,281. In some aspects, the mRNA can comprise one or more modifications that confer stability to the mRNA (e.g., compared to a wild-type or native version of the mRNA) and can also comprise one or more modifications relative to the wild-type which correct a defect implicated in the associated aberrant expression of the protein. For example, the nucleic acids of the disclosure can comprise modifications to one or both of the 5′ and 3′ untranslated regions. Such modifications can include, but are not limited to, the inclusion of a partial sequence of a cytomegalovirus (CMV) immediate-early 1 (IE1) gene, a poly A tail, a Cap1 structure or a sequence encoding human growth hormone (hGH)). In some aspects, the mRNA is modified to decrease mRNA immunogenecity.

In some aspects, the gene therapy is delivered by a transfer vehicle. In some aspects, the transfer vehicle is a liposomal transfer vehicle, e.g., a lipid nanoparticle, as described in U.S. Pat. No. 9,308,281. In some aspects, the mRNA encoding a human GLA protein or agalsidase alpha and/or beta is formulated in a liposomal transfer vehicle to facilitate delivery to the target cell. Contemplated transfer vehicles can comprise one or more cationic lipids, non-cationic lipids, and/or PEG-modified lipids. For example, the transfer vehicle can comprise at least one of the following cationic lipids: C12-200, DLin-KC2-DMA, DODAP, HGT4003, ICE, HGT5000, and HGT5001. In some aspects, the transfer vehicle comprises cholesterol (chol) and/or a PEG-modified lipid. In some aspects, the transfer vehicles comprises DMG-PEG2K. In some aspects, the transfer vehicle comprises one of the following lipid formulations: C12-200, DOPE, chol, DMG-PEG2K; DODAP, DOPE, cholesterol, DMG-PEG2K; HGT5000, DOPE, chol, DMG-PEG2K, HGT5001, DOPE, chol, and DMG-PEG2K.

In some aspects, the pre-treatment ERT comprises administering Galafold® (migalastat; Amicus Therapeutics). Galafold® is an alpha-galactosidase A (alphα-Gal A) pharmacological chaperone. In some aspects, 123 mg of Galafold® is administered orally once every other day at the same time of day, as described in e.g., Lenders et al., J Am Soc Nephrol, 29:2265-2278 (2018).

### V.B Enzyme Replacement Therapy and an Active Site-Specific Chaperone

In some aspects, the pre-treatment ERT of the disclosure comprises an α-Galactosidase A protein (e.g., recombinant α-Gal A (rhα-Gal A)) in combination with an active site-specific chaperone (ASSC) for the α-Gal A, e.g., migalastat (1-deoxygalactonojirimycin (DGJ)), as described in e.g., U.S. Pat. No. 10,155,027.

In some aspects, the disclosure provides for combination therapy of α-Gal A (e.g. rhα-Gal A ERT) and an ASSC for the α-Gal A enzyme (e.g., (DGJ)). In some aspects, the α-Gal A and ASSC are co-formulated together and administered to a subject concurrently as a co-formulation. In some aspects, the ASSC 1-deoxygalactonojirimycin is co-formulated with α-Gal A as a pharmaceutical composition. Such a composition can enhance stability of α-Gal A both during storage (i.e., in vitro) and in vivo after administration to a subject, thereby increasing circulating half-life, tissue uptake, and resulting in increased therapeutic efficacy of α-Gal A (e.g., increasing the reduction of tissue GL-3 levels). In some aspects, the route of administration is intravenous. Administration can be by periodic injections of a bolus of the preparation, or as a sustained release dosage form over long periods of time, such as by intravenous administration, for example, from a reservoir which is external (e.g., an IV bag).

The co-formulation suitable for intravenous administration use include sterile aqueous solutions or dispersions and sterile powders for the extemporaneous preparation of sterile injectable solutions or dispersion. In some aspects, the form is sterile and fluid to the extent that easy syringability exists. In some aspects, it is stable under the conditions of manufacture and storage and preserved against the contaminating action of microorganisms such as bacteria and fungi. In some aspects, the co-formulation comprises a carrier such as a solvent or dispersion medium containing, for example, water, ethanol, polyol (e.g., glycerol, propylene glycol, and polyethylene glycol, and the like), suitable mixtures thereof, and vegetable oils. The proper fluidity can be maintained, for example, by the use of a coating such as lecithin, by the maintenance of the required particle size in the case of dispersion, and by the use of surfactants. The preventions of the action of microorganisms can be brought about by various antibacterial and antifungal agents (e.g., parabens, chlorobutanol, phenol, benzyl alcohol, sorbic acid, and the like).

In some aspects, isotonic agents, for example, sugars or sodium chloride are added. Prolonged absorption of the injectable compositions can be brought about by the use in the compositions of agents delaying absorption, for example, aluminum monosterate and gelatin. Sterile injectable solutions can be prepared by incorporating the α-Gal A and ASSC (e.g., DGJ) in the required amounts in the appropriate solvent with various other ingredients enumerated above, as required, followed by filter or terminal sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

In some aspects, the co-formulation can contain an excipient. Pharmaceutically acceptable excipients which can be included in the co-formulation are buffers such as citrate buffer, phosphate buffer, acetate buffer, and bicarbonate buffer, amino acids, urea, alcohols, ascorbic acid, phospholipids; proteins, such as serum albumin, collagen, and gelatin, salts (such as EDTA, EGTA, and sodium chloride), liposomes, polyvinylpyrollidone, sugars (such as dextran, mannitol, sorbitol, and glycerol), propylene glycol, and polyethylene glycol (e.g., PEG-4000, PEG-6000), glycerol, glycine (or other amino acids), and lipids. Buffer systems for use with the co-formulations can include citrate, acetate, bicarbonate, and phosphate buffers.

The co-formulation can also contain a non-ionic detergent. Non-ionic detergents include, but are not limited to, Polysorbate 20, Polysorbate 80, Triton X-100, Triton X-114, Nonidet P-40, Octyl α-glucoside, Octyl β-glucoside, Brij 35, Pluronic, and Tween 20.

For lyophilization of protein and chaperone preparations, the protein concentration can be about 0.1 mg/mL to about 10 mg/mL. Bulking agents, such as glycine, mannitol, albumin, and dextran, can be added to the lyophilization mixture. In addition, possible cryoprotectants, such as disaccharides, amino acids, and PEG, can be added to the lyophilization mixture. Any of the buffers, excipients, and detergents listed above, can also be added.

In some aspects, the co-formulation comprises α-Gal A at a concentration of between about 0.05 and about 100 μM, between about 0.1 and about 75 μM, between about 0.2 and about 50 μM, between about 0.3 and about 40 μM, between about 0.4 and about 30 μM, between about 0.5 and about 20 μM, between about 0.6 and about 15 μM, between about 0.7 and about 10 μM, between about 0.8 and about 9 μM, between about 0.9 and about 8 μM, between about 1 and about 7 μM, between about 2 and about 6 μM, or between about 3 and about 5 μM.

In some aspects, the co-formulation comprises α-Gal A at a concentration of about 0.1, 0.2, 0.3, 0.4, 0.5, 0.6, 0.7, 0.8, 0.9, 1, 1.1, 1.2, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, or 15 μM.

In some aspects, the co-formulation comprises α-Gal A at a concentration of between about 0.0025 and about 5 mg/ml, between about 0.005 and about 4.5 mg/ml, between about 0.025 and about 4 mg/ml, between about 0.05 and about 3.5 mg/ml, between about 0.25 and about 3 mg/ml, between about 0.5 and about 2.5 mg/ml, between about 0.75 and about 2 mg/ml, or between about 1 and about 1.5 mg/ml.

In some aspects, the co-formulation comprises DGJ at a concentration of between about 10 and about 25,000 μM, between about 50 and about 20,000 μM, between about 100 and about 15,000 μM, between about 150 and about 10,000 μM, between about 200 and about 5,000 μM, between about 250 and about 1,500 M, between about 300 and about 1,000 μM, between about 350 and about 550 μM, or between about 400 and about 500 μM.

In some aspects, the co-formulation comprises DGJ at a concentration of between about 0.002 and about 5 mg/ml, between about 0.005 and about 4.5 mg/ml, between about 0.02 and about 4 mg/ml, between about 0.05 and about 3.5 mg/ml, between about 0.2 and about 3 mg/ml, between about 0.5 and about 2.5 mg/ml, or between about 1 and about 2 mg/ml.

In some aspects, the co-formulation comprises DGJ at a concentration of about 50, 100, 150, 200, 250, 300, 350, 400, 450, 500, 550, 600, 650, 700, 750, 800, 850, 900, 950, 1000, 2000, 3000, 4000, 5000, 6000, 7000, 8000, 9000, 10000, 11000, 12000, 13000, 14000, 15000, 16000, 17000, 18000, 19000, or 20000 μM.

In some aspects, the α-Gal A enzyme and DGJ are combined to create a co-formulation for administration to a subject, wherein the dosage of α-Gal A enzyme of the co-formulation administered to the subject is between about 0.05 and about 10 mg/kg, between about 0.1 and about 5 mg/kg, between about 0.2 and about 4 mg/kg, between about 0.3 and about 3 mg/kg, between about 0.4 and about 2 mg/kg, between about 0.5 and about 1.5 mg/kg, or between about 0.5 and about 1 mg/kg.

In some aspects, the dosage of α-Gal A enzyme of the co-formulation administered to the subject is about 0.05, 0.1, 0.2, 0.3, 0.4, 0.5, 1, 1.5, 2, 2.5, 3, 3.5, 4, 4.5, or 5 mg/kg.

In some aspects, the α-Gal A enzyme and DGJ are combined to create a co-formulation for administration to a subject, wherein the dosage of DGJ of the co-formulation administered to the subject is between about 0.05 and 20 mg/kg, between about 0.1 and about 15 mg/kg, between about 0.2 and about 10 mg/kg, between about 0.3 and about 10 mg/kg, between about 0.4 and about 9 mg/kg, between about 0.5 and about 8 mg/kg, between about 0.6 and about 7 mg/kg, between about 0.7 and about 6 mg/kg, between about 0.8 and about 5 mg/kg, between about 0.9 and about 4 mg/kg, between about 1 and about 3 mg/kg, or between about 1.5 and about 2 mg/kg.

In some aspects, the dosage of DGJ of the co-formulation administered to the subject is about 0.05, 0.1, 0.2, 0.3, 0.4, 0.5, 0.6, 0.7, 0.8, 0.9, 1, 2, 3, 4, 5, 6, 7, 8, 9 or 10 mg/kg.

In some aspects, the co-formulation of α-Gal A and DGJ can be administered intravenously to a subject in an amount effective to achieve a plasma AUC concentration of between about 0.5 and 10-fold, between about 1 and about 8-fold, between about 1.5 and about 6-fold, between about 2 and about 5.5-fold, between about 2.5 and about 5-fold, or between about 3 and about 4.5-fold of the plasma AUC concentration achieved when α-Gal A is administered to a subject in the same dosage as the co-formulation, but in the absence of DGJ.

As used herein, the term “AUC” represents a mathematical calculation to evaluate the body's total exposure over time to a given drug. In a graph plotting how concentration in the blood after dosing, the drug concentration variable lies on the y-axis and time lies on the x-axis. The area between a drug concentration curve and the x-axis for a designated time interval is the AUC. AUCs are used as a guide for dosing schedules and to compare different drugs' availability in the body.

In some aspects, the co-formulation of α-Gal A and DGJ can be administered intravenously to a subject in an amount effective to achieve a level of α-Gal A tissue uptake of between about 0.5 and 10-fold, between about 1 and about 8-fold, between about 1.5 and about 6-fold, between about 2 and about 5.5-fold, between about 2.5 and about 5-fold, or between about 3 and about 4.5-fold of the level of α-Gal A tissue uptake achieved when α-Gal A is administered to a subject in the same dosage as the co-formulation, but in the absence of DGJ.

Delivery of the co-formulation can be continuous over a pre-selected administration period ranging from several hours, one to several weeks, one to several months, or up to one or more years. In some aspects, the dosage form is one that is adapted for delivery of α-Gal A over an extended period of time. Such delivery devices can be adapted for administration of α-Gal A for several hours (e.g., 2 hours, 12 hours, or 24 hours to 48 hours or more), to several days (e.g., 2 to 5 days or more, from about 100 days or more), to several months or years. In some aspects, the device is adapted for delivery for a period ranging from about 1 month to about 12 months or more. The α-Gal A delivery device can be one that is adapted to administer α-Gal A to an individual for a period of, e.g., from about 2 hours to about 72 hours, from about 4 hours to about 36 hours, from about 12 hours to about 24 hours; from about 2 days to about 30 days, from about 5 days to about 20 days, from about 7 days to about 100 days or more, from about 10 days to about 50 days; from about 1 week to about 4 weeks; from about 1 month to about 24 months or more, from about 2 months to about 12 months, from about 3 months to about 9 months, or other ranges of time, including incremental ranges, within these ranges, as needed.

In some aspects, a dose of α-Gal A present in a co-formulation with DGJ is the intravenously administered once per day, once every two days, once every three days, once every four days, once every five days, or once every six days. In some aspects, the dose does not result in a toxic level of α-Gal A in the liver of the individual. In some aspects, the co-formulation composition of α-Gal A and DGJ is administered in a sufficient dose to result in a peak concentration of α-Gal A in tissues of the subject, within about 24 hours after the administration of the dose. In some aspects, the co-formulation composition is administered in a sufficient dose to result in a peak concentration of α-Gal A in tissues of the subject within between about 0.2 to about 50 hours, between about 0.2 to about 24 hours, between about 0.2 to about 5 hours, between about 0.2 to about 1 hour, between about 0.2 to about 0.5 hour, or about 40, 30, 20, 10, 5, 1, 0.5 or fewer hours after the administration of the dose. In some aspects, the co-formulation is administered as a single-dose. In some aspects, the co-formulation is administered as a multi-dose.

### V.C Non-Enzyme Replacement Therapy

In some aspects, the therapy for Fabry disease is an enzyme replacement therapy.

In some aspects, the non-enzyme replacement therapy comprises small molecule therapy. Some emerging drug development strategies for small molecule therapy of Fabry disease include but are not limited to substrate reduction therapy (SRT), residual enzyme activation, GLA promoter activation, protein homeostasis regulation (proteostasis), and chemical chaperone therapy (CCT), as described in e.g., Motabar et al., Curr Chem Genomics 4: 50-56 (2010).

In some aspects, small molecule therapy comprises administering lucerastat (Idorsia Pharmaceuticals Ltd), venglustat (Sanofi Genzyme), or apabetalone (development codes RVX 208, RVX-208, and RVX000222; Resverlogix Corp.), or any combination thereof.

### VI. Methods for Producing AAV Expression Vectors

Also within the scope of the present disclosure are methods for the generation of AAV particles, by viral genome replication in a viral replication cell comprising contacting the viral replication cell with an AAV polynucleotide or AAV genome (e.g., an AAV vector of the present disclosure). In the context of the present disclosure, the AAV expression vectors disclosed herein, are considered AAV payload construct vectors.

In some aspects, an AAV particle is produced by a method comprising the steps of: (1) co-transfecting competent bacterial cells with a bacmid vector and either a viral construct vector and/or AAV payload construct vector, (2) isolating the resultant viral construct expression vector and AAV payload construct expression vector and separately transfecting viral replication cells, (3) isolating and purifying resultant payload and viral construct particles comprising viral construct expression vector or AAV payload construct expression vector, (4) co-infecting a viral replication cell with both the AAV payload and viral construct particles comprising viral construct expression vector or AAV payload construct expression vector, and (5) harvesting and purifying the viral particle comprising a parvoviral genome.

In one aspect, the present disclosure provides a method for producing an AAV particle comprising the steps of (1) simultaneously co-transfecting mammalian cells, such as, but not limited to HEK293 cells, with a payload region (e.g., polynucleotide encoding a therapeutic molecule of the present disclosure), a construct expressing rep and cap genes and a helper construct, and (2) harvesting and purifying the AAV particle comprising a viral genome.

In some aspects, the AAV particles can be produced in a viral replication cell that comprises an insect cell. Growing conditions for insect cells in culture, and production of heterologous products in insect cells in culture are well-known in the art, see, e.g., U.S. Pat. No. 6,204,059.

The viral replication cell can be selected from any biological organism, including prokaryotic (e.g., bacterial) cells, and eukaryotic cells, including, insect cells, yeast cells and mammalian cells. Viral replication cells can comprise mammalian cells such as A549, WEH1, 3T3, 10T1/2, BHK, MDCK, COS 1, COS 7, BSC 1, BSC 40, BMT 10, VERO.

W138, HeLa, HEK293, Saos, C2C12, L cells, HT1080, HepG2 and primary fibroblast, hepatocyte and myoblast cells derived from mammals. Viral replication cells comprise cells derived from mammalian species including, but not limited to, human, monkey, mouse, rat, rabbit, and hamster or cell type, including but not limited to fibroblast, hepatocyte, tumor cell, cell line transformed cell, etc.

Viral production disclosed herein describes processes and methods for producing AAV particles that contact a target cell to deliver a payload, e.g. a recombinant viral construct, which comprises a polynucleotide sequence encoding a payload such, for example, as an α-Gal A protein.

In some aspects, the AAV particles can be produced in a viral replication cell that comprises a mammalian cell. Viral replication cells commonly used for production of recombinant AAV particles include, but are not limited to 293 cells, COS cells, HeLa cells, and KB cells.

In some aspects, AAV particles are produced in mammalian cells wherein all three VP proteins are expressed at a stoichiometry approaching 1:1:10 (VP1:VP2:VP3). The regulatory mechanisms that allow this controlled level of expression include the production of two mRNAs, one for VP1, and the other for VP2 and VP3, produced by differential splicing.

In some aspects, AAV particles are produced in mammalian cells using a triple transfection method wherein a payload construct, parvoviral Rep and parvoviral Cap and a helper construct are comprised within three different constructs. The triple transfection method of the three components of AAV particle production can be utilized to produce small lots of virus for assays including transduction efficiency, target tissue (tropism) evaluation, and stability.

In some aspects, the viral construct vector and the AAV payload construct vector can be each incorporated by a transposon donor/acceptor system into a bacmid, also known as a baculovirus plasmid, by standard molecular biology techniques known and performed by a person skilled in the art. Transfection of separate viral replication cell populations produces two baculoviruses, one that comprises the viral construct expression vector, and another that comprises the AAV payload construct expression vector. The two baculoviruses can be used to infect a single viral replication cell population for production of AAV particles.

Baculovirus expression vectors for producing viral particles in insect cells, including but not limited to Spodoptera frugiperda (Sf9) cells, provide high titers of viral particle product. Recombinant baculovirus encoding the viral construct expression vector and AAV payload construct expression vector initiates a productive infection of viral replicating cells. Infectious baculovirus particles released from the primary infection secondarily infect additional cells in the culture, exponentially infecting the entire cell culture population in a number of infection cycles that is a function of the initial multiplicity of infection, see, e.g., Urabe, M. et al., J Virol. 2006 February; 80 (4): 1874-85, the contents of which are herein incorporated by reference in their entirety. In some aspects, the AAV expression vector of the present disclosure (e.g., the AAV-001 rAAV vector) comprises the α-Gal A expression cassette flanked on each end by ITRs derived from AAV2, wherein the α-Gal A expression cassette is packaged with capsid derived from adeno-associated virus type 6 (AAV6) using a Sf9 insect cell/recombinant baculovirus (Sf9/rBV) expression system. In some aspects, the AAV expression vector of the present disclosure (e.g., the AAV-001 rAAV vector) comprises the α-Gal A expression cassette flanked on each end by ITRs derived from AAV2, wherein the α-Gal A expression cassette is packaged with capsid derived from AAV6 using a mammalian expression system, e.g., HEK293.

Production of AAV particles with baculovirus in an insect cell system can address known baculovirus genetic and physical instability. Baculovirus-infected viral producing cells are harvested into aliquots that can be cryopreserved in liquid nitrogen; the aliquots retain viability and infectivity for infection of large-scale viral producing cell culture (Wasilko D J et al., Protein Expr Purif. 2009 June; 65(2): 122-32).

In some aspects, stable viral replication cells permissive for baculovirus infection are engineered with at least one stable integrated copy of any of the elements necessary for AAV replication and viral particle production including, but not limited to, the entire AAV genome, Rep and Cap genes, Rep genes, Cap genes, each Rep protein as a separate transcription cassette, each VP protein as a separate transcription cassette, the AAP (assembly activation protein), or at least one of the baculovirus helper genes with native or non-native promoters.

In some aspects, AAV particle production can be modified to increase the scale of production. Transfection of replication cells in large-scale culture formats can be carried out according to any methods known in the art.

In some aspects, cell culture bioreactors can be used for large scale viral production. In some cases, bioreactors comprise stirred tank reactors.

### Cell Lysis

Cells of the disclosure, including, but not limited to viral production cells, can be subjected to cell lysis according to any methods known in the art. Cell lysis can be carried out to obtain one or more agents (e.g. viral particles) present within any cells of the disclosure.

Cell lysis methods can be chemical or mechanical. Chemical cell lysis typically comprises contacting one or more cells with one or more lysis agent. Mechanical lysis typically comprises subjecting one or more cells to one or more lysis condition and/or one or more lysis force. In some aspects, chemical lysis can be used to lyse cells. As used herein, the term “lysis agent” refers to any agent that can aid in the disruption of a cell. In some cases, lysis agents are introduced in solutions, termed lysis solutions or lysis buffers. As used herein, the term “lysis solution” refers to a solution (typically aqueous) comprising one or more lysis agent. In addition to lysis agents, lysis solutions can include one or more buffering agents, solubilizing agents, surfactants, preservatives, cryoprotectants, enzymes, enzyme inhibitors and/or chelators.

Concentrations of salts can be increased or decreased to obtain an effective concentration for rupture of cell membranes. Lysis agents comprising detergents can include ionic detergents or non-ionic detergents. Detergents can function to break apart or dissolve cell structures including, but not limited to cell membranes, cell walls, lipids, carbohydrates, lipoproteins and glycoproteins.

In some aspects, mechanical cell lysis is carried out. Mechanical cell lysis methods can include the use of one or more lysis condition and/or one or more lysis force. As used herein, the term “lysis condition” refers to a state or circumstance that promotes cellular disruption. Lysis conditions can comprise certain temperatures, pressures, osmotic purity, salinity and the like. In some aspects, lysis conditions comprise increased or decreased temperatures. In some aspects, lysis conditions comprise changes in temperature to promote cellular disruption. Cell lysis carried out according to such aspects can include freeze-thaw lysis.

As used herein, the term “lysis force” refers to a physical activity used to disrupt a cell. Lysis forces can include, but are not limited to mechanical forces, sonic forces, gravitational forces, optical forces, electrical forces and the like. Cell lysis carried out by mechanical force is referred to herein as “mechanical lysis.” Mechanical forces that can be used according to mechanical lysis can include high shear fluid forces.

In some aspects, a method for harvesting AAV particles without lysis can be used for efficient and scalable AAV particle production. In a non-limiting example, AAV particles can be produced by culturing an AAV particle lacking a heparin binding site, thereby allowing the AAV particle to pass into the supernatant, in a cell culture, collecting supernatant from the culture; and isolating the AAV particle from the supernatant, as described in U.S. patent application No. 20090275107.

### AAV Purification

Cell lysates comprising viral particles can be subjected to clarification. Clarification refers to initial steps taken in purification of viral particles from cell lysates. Clarification serves to prepare lysates for further purification by removing larger, insoluble debris. Clarification steps can include, but are not limited to centrifugation and filtration.

In some aspects, AAV particles can be purified from clarified cell lysates by one or more methods of chromatography. Chromatography refers to any number of methods known in the art for separating out one or more elements from a mixture. Such methods can include, but are not limited to ion exchange chromatography (e.g. cation exchange chromatography and anion exchange chromatography), immunoaffinity chromatography and size-exclusion chromatography.

All of the references cited above, as well as all references cited herein, are incorporated herein by reference in their entireties.

The following examples are offered by way of illustration and not by way of limitation.

### EXAMPLES

While the present disclosure has been described with reference to the specific aspects thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the disclosure.

### Example 1

Clinical Evaluation of AAV-001 in Adults with Fabry Disease

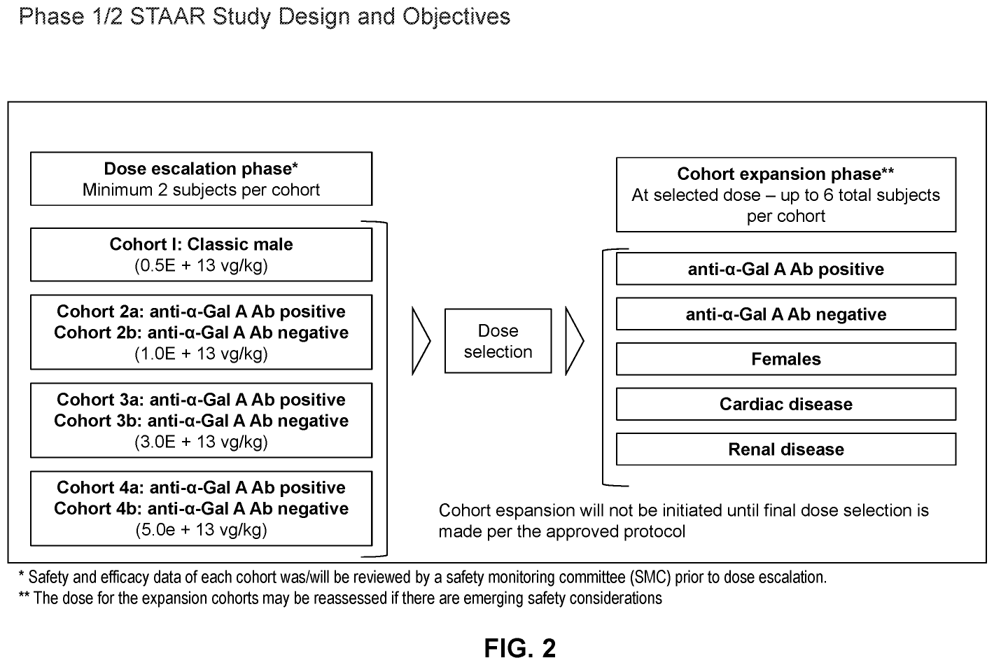

AAV-001 is assessed in a phase 1/2, multicenter, open-label, single-dose, dose-ranging study to assess the safety and tolerability of AAV-001, a rAAV2/6 human α-Gal A gene therapy in subjects with Fabry disease. The study design is presented in FIG. 2.

The primary objective of the study is to assess safety and tolerability of AAV-001. The secondary objective of the study is assess the pharmacodynamics of α-Gal A and the presence of its substrates in plasma over time; to assess impact of AAV-001 on ERT administration required for subjects on ERT; to assess the impact of AAV-001 on renal and cardiac function; to assess clinical impact of AAV-001 on Fabry disease (including quality of life (QoL)), and to evaluate rAAV2/6 vector DNA shedding over time. The additional objective of the study is to assess the pharmacodynamics of α-Gal A and the presence of its substrates in urine and tissue over time; to assess the pharmacokinetics of α-Gal A over time; and to assess immune response to rAAV2/6 and α-Gal A.

### Study Evaluations

Evaluations include incidents of treatment-emergent adverse events (TEAEs), routine hematology, chemistry, and liver function, vital signs, ECO and ECHO, serial alpha fetoprotein (AFP) testing and MRI of liver (or equivalent imaging) to monitor for the formation of any liver mass. Additionally, the change from baseline can be measured at specific time points over 1 year in the following: α-Gal A activity in plasma; Gb3 levels in plasma; Lyso-Gb3 levels in plasma; frequency of FABRAZYME® (or equivalent ERT) infusion; estimated glomerular filtration rate (eGFR) calculated by creatinine levels in blood; left ventricular mass measured by cardiac magnetic resonance imaging (MRI), total protein and albumin to creatinine ratios in urine; α-Gal A and Gb3 levels measured in tissue; substrate levels measured in tissues and urine; biomarkers of renal function in urine; neuropathic pain measured by the Brief Pain Inventory (BPI), frequency of pain medication use; gastrointestinal (GI) symptoms measured by the GI symptoms rating scale; Mainz Severity Score Index (MSSI); quality of life (QOL) patient-reported outcome measured by the SF-36 questionnaire; immune response to rAAV2/6 and α-Gal A; and rAAV vector clearance can be measured by level of vector genome in blood, plasma, saliva, urine, stool, and semen.

### Target Subject Population and Eligibility Criteria

Male and female subjects ≥18 years of age with classical Fabry disease. The subjects can be on enzyme replacement therapy (ERT) regimen; or ERT-naïve; or ERT-pseudo-naïve and has not received ERT treatment in the prior six months, as described herein.

The subject inclusion criteria comprises: (1) subjects with documented diagnosis of classical Fabry disease as defined by <5% α-Gal A activity in either plasma or leukocytes and one or more of the following symptomatic characteristics of classical Fabry disease: i) cornea verticillata, ii) acroparesthesia, iii) anhidrosis, iv) angiokeratoma (if there is documented clustered periumbilical angiokeratoma, this symptom alone is sufficient as it is a pathognomonic sign of classical Fabry disease); (2) subject who is on ERT (14 days [±1 day] regimen); or subject on ERT whose-Gal A activity is >5%; or is ERT-nalve; or is ERT-pseudo-naave and has not received ERT treatment in the past 6 months prior to consent; (3) for subjects receiving ERT, ERT should have been administered at a stable dose (defined as not having missed more than 3 doses of ERT during the 6 months prior to consent) and regimen (14 days [±1 day] for at least 3 months prior to enrollment); (4) subject with a mutation that is indicative of classical Fabry (i.e., listed in a database, such as International Fabry Disease Genotype-Phenotype Database (dbFGP)); (5) subject whose α-Gal A activity at trough is below the lower limit of the normal range of the assay; (6) subjects >about 18 years of age; (7) sexually mature subjects must agree to use a condom and refrain from sperm donation from the time of expression construct administration until a minimum of 3 consecutive semen samples are negative for AAV after administration of study treatment and a minimum of 90 days after study treatment administration; and (8) signed, written informed consent of the subject.

For subjects who do not have a documented diagnostic α-Gal A activity level, a blood sample is taken to measure α-Gal A activity levels (in plasma and/or leukocytes). For those subjects who are on ERT, this blood draw is taken at least 13 days after their last ERT infusion (trough), i. If the subject's level of α-Gal A activity is >5% and the subject is on ERT, this level of enzyme activity can be due to residual α-Gal A activity from the last ERT infusion. In this case, the diagnosis of classical Fabry disease is confirmed if the following three criteria are fulfilled:
a. two or more of the following documented symptomatic characteristics of classical Fabry: cornea verticillata, acroparesthesia, anhidrosis, angiokeratoma. If there is documented clustered periumbilical angiokeratoma, this symptom alone is sufficient as it is a pathognomonic sign of classical Fabry disease;
b. a mutation that is indicative of classical Fabry (i.e. listed in a database, such as www.dbfgp.org); and
c. the α-Gal A activity at trough is below the lower limit of the normal range of the assay.

Fabry disease gene sequencing is performed at screening to confirm that subjects have a mutation in the α-Gal A gene. The assay is performed on blood or saliva samples. If available, gene sequencing results obtained prior to the study can be used. Testing for HIV, HAV, HBV, HCV, and TB is conducted at screening. Subjects with a diagnosis of HIV or evidence of active HAV, HBV, HCV, or TB infection may not be eligible to participate in this study.

The level of neutralizing antibodies to AAV6 is measured at screening to assess the subject's pre-existing immune response to AAV6. Subjects with elevated pre-existing neutralizing antibodies to AAV6 may not be eligible to participate in this study. If dosing is not completed within 3 months of screening, the serum neutralization assay to AAV6 is repeated.

If available, diagnostic α-Gal A activity level results in plasma or leukocytes obtained prior to the study are used. For subjects who do not have a documented diagnostic α-Gal A activity level, a blood sample is taken to measure α-Gal A activity levels (in plasma and/or leukocytes). For those subjects who are on ERT, this blood draw is taken at least 13 days after their last ERT infusion.

Chest X-rays (also known as PA radiograph of the chest) can be obtained to evaluate the general health and study eligibility of the subject. Unless medically indicated, a chest X-Ray taken within 6 months of enrollment in the study is used to determine subject eligibility. Physical examinations is conducted on each subject and include at minimum: general appearance, head, eyes, ears, nose, and throat (HEENT); as well as cardiovascular, dermatologic, respiratory, GI, musculoskeletal, and neurologic systems.

The subject exclusion criteria can comprise subjects who: (1) are known to be unresponsive to ERT in the opinion of the Site Investigator and Medical Monitor (e.g., no documented substrate level decrease on ERT); (2) are undergoing current treatment with migalastat (Galafold™) or prior treatment within 3 months of informed consent, (3) have a positive neutralizing antibody response to AAV (e.g., AAV6), (4) have intercurrent illness expected to impair evaluation of safety or efficacy during the observation period of the study in the opinion of the Site Investigator or Medical Monitor; (5) have eGFR <60 ml/min/1.73 m2; (6) have a New York Heart Association Class PI or higher; (7) have an active infection with hepatitis A virus (HAV), hepatitis B virus (HBV), hepatitis C virus (HCV) (negative HCV-DNA), or human immunodeficiency virus (HIV) as measured by quantitative polymerase drain reaction (qPCR) or active infection with tuberculosis (TB); (8) have a history of liver disease such as secondary steatosis, non-alcoholic steatohepatitis (NASH) and cirrhosis, cholangitis, biliary disease within 6 months of informed concent; except for Gilbert's syndrome; abnormal circulating AFP; (9) for subjects receiving ERT, have recent or continued hypersensitivity response to ERT treatment within 6 months prior to consent, as manifested by significant infusion reaction to ERT in tire opinion of the Site Investigator and Medical Monitor; (10); markers of hepatic inflammation or overt or occult causes of liver dysfunction as confirmed by one or more of the following: (i) albumin <3.5 g/dL; (ii) total bilirubin >upper limit of normal (ULN) and direct bilirubin >0.5 mg/dL; (iii) alkaline phosphatase (ALP) >2.0×ULN; (iv) alanine aminotransferase (ALT) >1.5×ULN; (11) have a current or history of systemic (IV or oral) immunomodulatory agent or steroid use in the past 6 months (topical treatment is allowed, e.g. asthma or eczema) (occasional use of systemic steroid may be allowed after discussion with the Medical Monitor); (12) have a contraindication to use of corticosteroids for immunosuppression; (13) have a history of malignancy except for non-melanoma skin cancer, (14) have a history of alcohol or substance abuse; (15) have participated in prior investigational interventional drug or medical device study within the last 3 months prior to consent (with the exception of implantable loop recorders as in the RalLRoAD trial); (16) have received prior treatment with a gene therapy product; (17) known hypersensitivity to components of AAV-001 formulation; (18) any other reason that, in the opinion of the Site Investigator or Medical Monitor, would raider the subject unsuitable for participation in the study.

### Baseline Period

Baseline assessments and procedures are performed within 12 weeks prior to AAV-001 infusion. In subjects receiving ERT, assessments including α-Gal A levels, and Gb3 and lyso-Gb3 will be taken at ERT trough levels, defined as 14 days (±1 day) after the previous ERT administration. Two samples will be taken at baseline on 2 different days in the morning. For subjects not on ERT, two samples will be taken at baseline on 2 occasions with 14 days (±1 day) between the 2 occasions.

### Concomitant Medications

All medications can be permitted, except for those that are potentially hepatotoxic. Hepatotoxic agents such as diclofenac, amiodarone, chloipromazine, fluconazole, isoniazid, rifampin, valproic acid, high doses of acetaminophen (4-8 gm/day), etc. as well as hepatotoxic herbal supplements such as senecio/crotal aria, germander in teas, chaparral, Jin bu huan, Ma-huang (Chinese hetbs), etc. should not be taken during the study period. For subjects receiving ERT, ERT should have been administered at a stable dose (defined as not having missed more than 3 doses of ERT during the past 6 months prior to concent) and regimen (14 days±1 day for at least 3 months prior to enrollment. Subjects should continue to receive ERT at a stable dose and regimen (14 days±1 day) during the study as per standard of care unless they undergo ERT withdrawal.

### Dose Cohorts

The dose considered to be tolerable and safe will be utilized in the dose expansion cohorts. After dose escalation has been completed, dose expansion will commence where up to 6 subjects will be enrolled into each of the dose expansion cohorts, which include patients with classical Fabry disease who are antibody-positive to α-Gal A, patients with classical Fabry disease who are antibody-negative to α-Gal A, female patients (female cohort) and patients who meet criteria for the renal (renal cohort) and cardiac (cardiac cohort) inclusion and exclusion criteria. The dose for the expansion cohorts may be reassessed if there are emerging safety considerations.

Anti α-Gal A Ab positive cohort: Up to 6 male subjects with classical Fabry disease who are antibody-positive to α-Gal A will be enrolled.

Anti α-Gal A Ab negative cohort: Up to 6 male subjects with classical Fabry disease who are antibody-negative to α-Gal A will be enrolled.

Female cohort: Up to 6 female subjects with classical Fabry disease will be enrolled.

Renal cohort: Up to 6 male or female subjects with symptomatic Fabry disease with a linear negative eGFR slope (estimated from at least 3 historical serum creatinine values [within 18 months, including the value obtained during screening visit]) of ≥2 mL/min/1.73 m²/year will be enrolled.

Cardiac cohort: Up to 6 male or female subjects with symptomatic Fabry disease with cardiac involvement, defined as either left ventricular hypertrophy (LVH) in 2D echocardiography or CMR (end diastolic septum and posterior wall thickness ≥12 mm) with no other explanation for LVH OR cardiac changes indicative of disease progression such as decreased global longitudinal strain on 2D strain echocardiography or low native T1 mapping on CMR will be enrolled.

Subjects who have had a cardiovascular event in the 6-month period before screening may be excluded at the discretion of the Investigator.

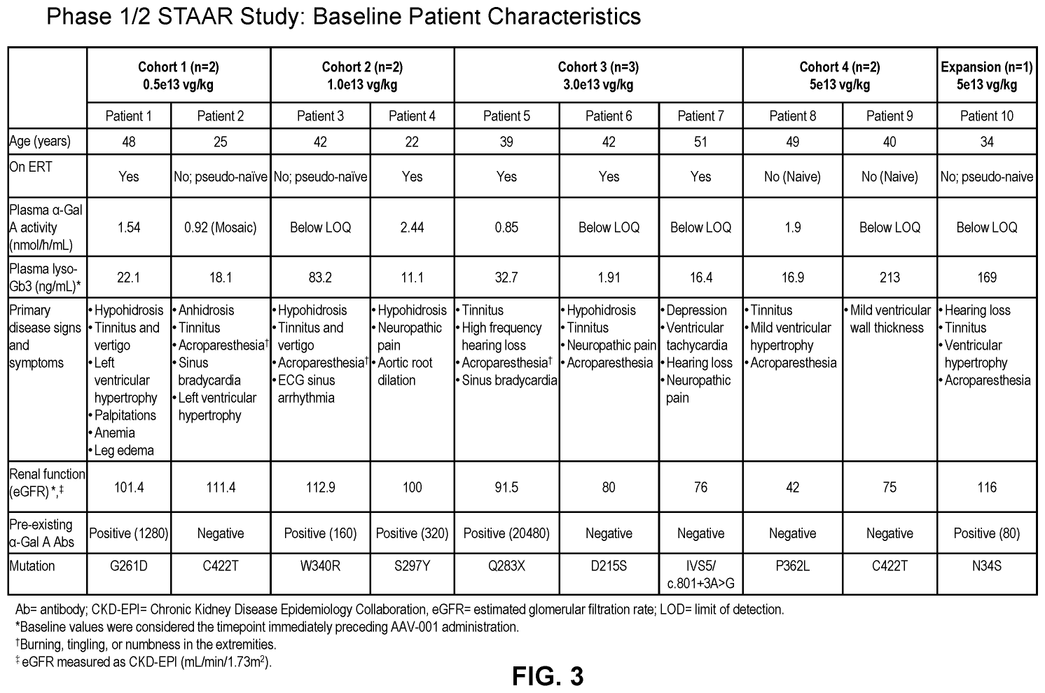

The starting dose is 5.0E+12 vg/kg, and any dose escalation to the next dose level is upon review of data from the previous cohort and/or other clinical trials that use in vivo rAAV2/6-based therapy, and based on the recommendation of the Safety Monitoring Committee (SMC), which comprises external subject matter experts, the study medical monitors, and site investigators as appropriate. As used herein, the SMC members have appropriate medical and scientific expertise and will provide safety oversight of the study. In addition, depending on the observed enzyme activity levels and safety profile of the subjects dosed, the SMC can recommend a dose escalation to an intermediate dose level of 3.0E+13 vg/kg in Cohort 3 (a 3-fold increase from the dose in Cohort 2) (instead of a 5-fold increase to the dose in Cohort 3. The protocol also allows for an additional dose level of 5.0E+13 vg/kg to be included as an additional dose in Cohort 4. However, no dose given to subjects will exceed 5.0E+13 without a substantial amendment. The dose cohorts are shown in Table 2. (See also FIG. 3)

TABLE 2
Dose by Cohorts

| Cohort # | Total rAAV* Dose (vector genomes [vg]/kg) |
|---|---|
| 1 | 5e12 vg/kg |
| 2 | 1.0e13 vg/kg |
| 3 | 3.0e13 vg/kg |
| 4 | 5.0e13 vg/kg |

*rAAV—recombinant adeno- associated virus

### Treatment Plan

Subjects >18 years of age who satisfy all inclusion/exclusion criteria are enrolled. At least two subjects were assigned into each of the dose cohorts with a potential expansion of any cohort with an additional 4 adult subjects, for a total of up to 18 subjects, after SMC review. The expression vector is administered via intravenous infusion. Within each cohort, treatment is staggered so that each subsequent subject cannot be infused until at least about 2 weeks after the preceding subject has been dosed. Dose escalation to the next dose level cannot occur until at least about 4 weeks after the last subject in the preceding cohort has been dosed, and safety data from the entire prior cohort has been reviewed by the SMC.

Subjects who received ERT prior to study enrollment should continue to receive ERT during the study and remain on their current dose and regimen (14 days±1 day) per standard of care unless they undergo ERT withdrawal. For subjects on ERT, baseline testing of enzyme and substrate levels are coordinated such that samples can be taken on 2 separate occasions in the morning at trough, defined as 14 days (+/−1 day) after the previous ERT infusion. An additional time point is taken previously during the screening period, therefore, having 3 time points to assess the residual levels of α-Gal A at trough prior to the gene therapy administration. These three samples are taken at trough, and preferably at the same time during the day (e.g. in the morning) to minimize non-specific factors potentially impacting on the levels of the enzymes.

To minimize the potential immune response to the rAAV capsid protein, to avoid losing transgene expression in the case of liver damage and to preserve hepatic function, prednisone or equivalent corticosteroid can be administered prophylactically starting about 2 days prior to expression vector infusion and can be tapered over a period of up to about 20 weeks.

The expression vector can be injected using a syringe pump or IV infusion pump (see Study Pharmacy Manual). Total volumes will be dependent on subject's cohort assignment and body weight (kg) at baseline. The expression vector can be administered through an IV catheter at a controlled speed while monitoring the subject's vital signs (temperature, heart rate, respiratory rate, and blood pressure) while the subject is in the hospital or acute care facility, where the subject may remain for observation for at least 24 hours after completion of the expression vector infusion. The subject can be discharged when all vital signs are stable and any adverse events (AEs) have resolved or the subject is considered stabilized as per the Investigator judgment.

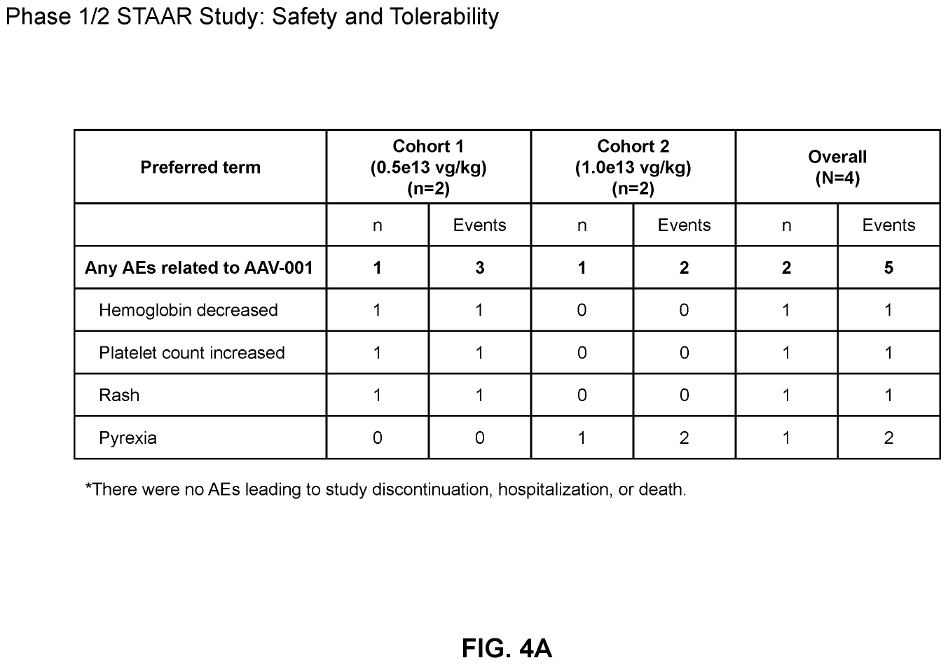

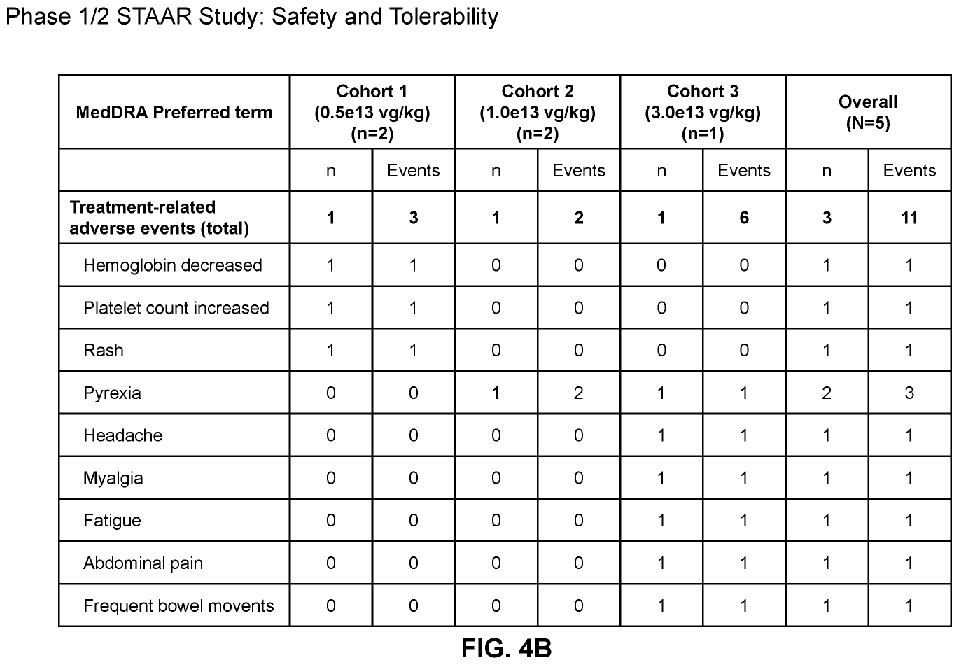

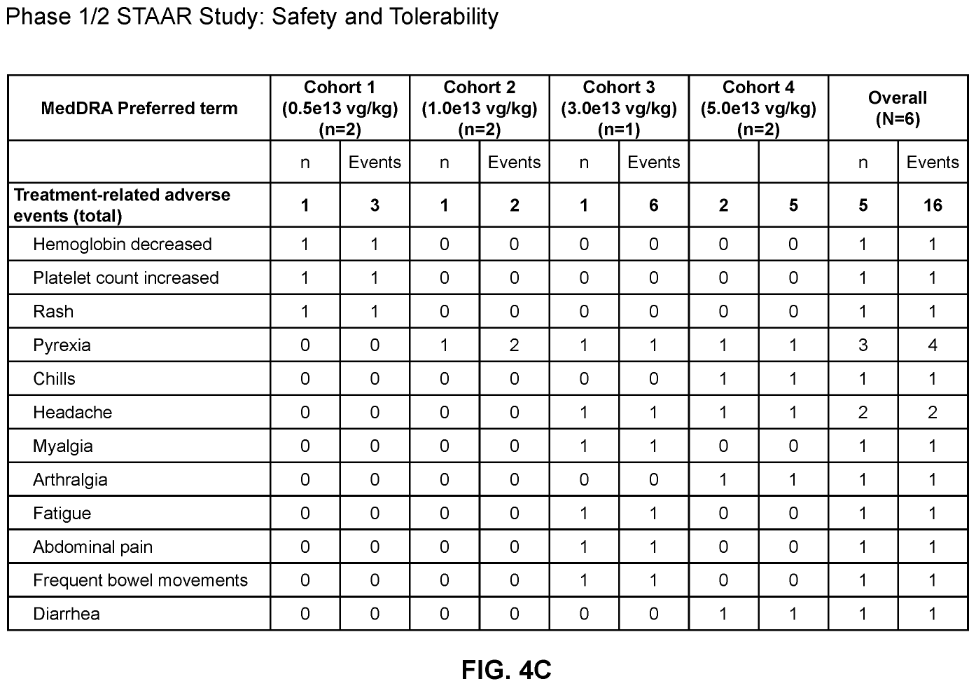

FIG. 4A shows safety data evaluated from the 4 patients in the first 2 dose cohorts (0.5e13 vg/kg and 1e13 vg/kg) as of the cutoff date. There were no liver enzyme elevations that required steroid treatment. There were no adverse events (AEs) leading to study discontinuation, hospitalization, or death.

FIG. 4B shows safety data evaluated from the five patients in dose cohorts 1-3 (0.5e13 vg/kg, 1.0e13 vg/kg, and 3.0e13 vg/kg) as of the cutoff date. There were no liver enzyme elevations that required steroid treatment. There were no adverse events (AEs) leading to study discontinuation, hospitalization, or death.

FIG. 4C shows safety data evaluated from the five patients in dose cohorts 1-4 (0.5e13 vg/kg, 1.0e13 vg/kg, 3.0e13 vg/kg and 5.0e 13 vg/kg) as of the cutoff date. There were no liver enzyme elevations that required steroid treatment. There were no adverse events (AEs) leading to study discontinuation, hospitalization, or death.

Following infusion with AAV-001, study visits can be conducted on Day 8; Weeks 2, 4, 6, 8, 12, 16, 20, 24, 28, 32, 36, 40, 44, 48, and 52. Week 28, 32, 40, 44, and 48 study visits have assessments that do not require evaluation at the clinical site, and therefore can be conducted remotely. Assessments for AEs and concomitant medications can be conducted remotely over the phone.

Liver tests (AST, ALT, GOT, total and direct bilirubin, ALP, LDH, albumin, and total protein levels) are conducted to monitor for AAV-mediated immunogenicity twice weekly during about the first 20 weeks after expression vector infusion while the subject is on prednisone or equivalent corticosteroid and may be conducted remotely. Blood samples for liver tests are drawn 2-4 days apart when possible, except for the first week when they can be drawn on the Day 2 and Day 8 visits. Liver tests can subsequently be conducted weekly for four weeks following discontinuation of immunosuppression (Weeks 21-24), and then monthly thereafter to coincide with study visits (Weeks 28-52).

If, despite pre-treatment with prednisone or equivalent corticosteroid, there is evidence of ALT elevation, the dose of prednisone or equivalent corticosteroid can be continued (prednisone 1 mg/kg [max 60 mg] or equivalent; oral or intravenous and/or increased on a case-by-case basis, and liver enzymes may be assessed twice a week until normalization of liver enzymes, and then per protocol thereafter.

### Dose Escalation

For the first 2 subject in each cohort, treatment is staggered so that each subsequent subject will not be infused until the preceding subject has been observed for at least 2 weeks. Dose escalation to the next dose level cannot occur until at least 4 weeks after 2 subjects in the preceding cohort has been dosed and the safety data from the 2 subjects in the prior cohort has been reviewed by the SMC. Dosing and dose escalation can be paused if any of the stopping rules are met.

### ERT Withdrawal

Treatment with AAV-001 could abrogate the need for ERT, by using a rAAV vector encoding cDNA for human α-Gal A, resulting in long-term, liver-specific expression of α-Gal A in Fabry disease subjects. Subjects who undergo ERT withdrawal are closely monitored for any AEs, vital signs, any changes in safety laboratory evaluations and levels of α-Gal A and substrates compared to baseline. The ERT withdrawal should be considered after a period of four weeks, in order to allow enough time for transduction of the target liver cells. The subjects who undergo ERT withdrawal are closely monitored for any clinical symptoms including fatigue, and neuropathic pain, any AEs, vital signs, any changes in safety laboratory evaluations, including liver function tests and levels of α-Gal A and substrates (Gb3 and Lyso-Gb3) compared to baseline. ERT withdrawal may be at the discretion of the Site Investigator after consultation with the Sponsor, and are considered for subjects who are willing and meet the following criteria:
(1) are >4 weeks post-administration of AAV-001;
(2) are medically stable and can tolerate temporary discontinuation of ERT in the judgment of the Site Investigator,
(3) agree to increased safety monitoring and additional lab testing until ERT Withdrawal Follow-Up visit;
(4) ERT does not need to be restarted after the ERT Withdrawal Follow-Up visit.

However, ERT may be re-initiated at any time based on clinical circumstances or at the judgment of the Site Investigator.

ERT withdrawal can be repeated if previously unsuccessful, provided this is done at least 12 weeks after the previous attempt if the subject is willing, and may be at the discretion of the Site Investigator and after consultation with the Sponsor.

### ERT Withdrawal Monitoring (±2 Days)

ERT Withdrawal Monitoring visits take place on a weekly basis for the first 4 weeks following the ERT Withdrawal visit, and then every other week for the last 8 weeks following the ERT Withdrawal visit until the ERT Withdrawal Follow-Up visit. ERT Withdrawal Monitoring visits should be combined with regular scheduled visits whenever possible to reduce study burden. ERT Withdrawal Monitoring visits can be conducted remotely.

### ERT Withdrawal Follow-Up (Up to 12 Weeks Post-ERT Withdrawal)

The ERT withdrawal follow-up visit will occur at Week 12 after the ERT withdrawal visit, but it can occur earlier at the discretion of the Site investigator, if clinically indicated. If clinically indicated, ERT may be re-initiated at any time based on clinical circumstances or at the judgment of the Site Investigator.

### End of Study Visit and Long-Term Follow-Up Study

An End of Study (EOS) visit will be conducted at Week 52 for final assessments. At the EOS visit, subjects will be encouraged to participate in a separate long-term follow-up study and subjects will be followed for up to 4 additional years.

### Study Duration

The duration of study participation can be up to 76 weeks for each subject divided into up to 8 weeks for screening, up to 12 weeks for baseline, and 52 weeks follow-up after dosing. Accrual is planned for 9 to 12 months.

### Safety Monitoring Committee & Stopping Rules

The study enrollment should be paused if any of the following criteria are met and the SMC may convene to make recommendations as to the proper course of action: (1) any one Grade 3 or higher adverse event with at least a reasonable possibility of a causal relationship to the expression vector formulation; (2) serious adverse event (SAE) with at least a reasonable possibility of a causal relationship to the expression vector formulation; (3) death of a human subject; (4) development of a malignancy.

The study may also be stopped for any of the following reasons: (1) Sponsor, in consultation with the SMC or Regulatory Agency, decides for any reason that subject safety may be compromised by continuing the study; (2) Sponsor decides to discontinue development of AAV-001.

All data will be evaluated to determine whether changes should be made to the study or if accrual should be continued or halted. If stopping criteria are met, no further dosing of subjects will be performed at that dose level or higher until a substantial amendment is submitted to regulatory authorities for review, and the amendment has been approved by the site Institutional Review Board (IRB)/Independent Ethics Committee (IEC) or equivalent.

### Example 2

### Plasma α-Gal A Activity and Lyso-Gb3

To test the effects of AAV-001 treatment in adults with Fabry disease plasma α-Gal A activity and lyso-Gb3 were evaluated from the 9 patients (patient 1, patient 2, patient 3, patient 4, patient 5, patient 6, patient 7, patient 8 and patient 9) across 4 dose cohorts (Cohort 1 (n=2; patients 1 and 2); 0.5e13 vg/kg and Cohort 2 (n=2; patients 3 and 4); 1e13 vg/kg, Cohort 3 (n=3; patients 5, 6 and 7); 3.0e13 vg/kg and Cohort 4 (n=2; patients 8 and 9); 5.0e13 vg/kg). Baseline characteristics for patients 1-9 are shown in FIG. 3.

### Plasma α-Gal A Activity

α-Gal A activity in K2-EDTA plasma was determined by measuring 4-methylumbelliferone (4-MU) product resulting from cleavage of an artificial substrate, 4-methylumbelliferyl α-D galactopyranoside (4-MU-α-Gal) at 37° C. as shown follows:

4-MU-α-Gal (substrate, non-fluorescent)+α-Gal A→4-MU (fluorescent)

The activity was represented by the amount of generated 4-MU (nmol/mL) in a 3-hour reaction time and presented in nmol/hr/mL.

### Normal Male Range for α-Gal A Plasma Activity and Fold Change

Normal male range for α-Gal A was determined from 40 healthy male donors using the validated plasma α-Gal A activity assay at 3-hour incubation time and presented in nmol/hr/mL. Male normal range: 2.45 to 11.37 nmol/h/mL, mean 5.70 nmol/h/mL. Fold change over mean was calculated in reference to the determined mean normal range at 5.70 nmol/h/mL.

### Normal Male Range for Lyso-Gb3:

Normal male range for lyso-Gb3 in K2-EDTA plasma was determined from 40 healthy male donors using a validate LC-MS/MS and lyso-Gb3-d7 as internal standard. The method utilized a protein precipitation extraction procedure prior to LC-MS/MS analysis. Male normal range: 0.323 to 0.625 ng/mL, mean concentration of 0.473 ng/mL.

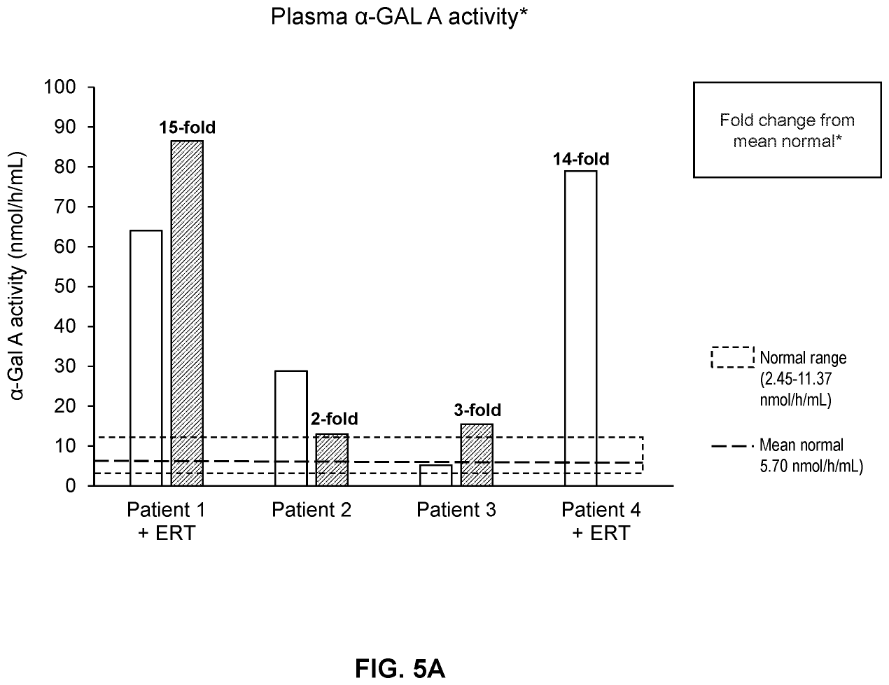

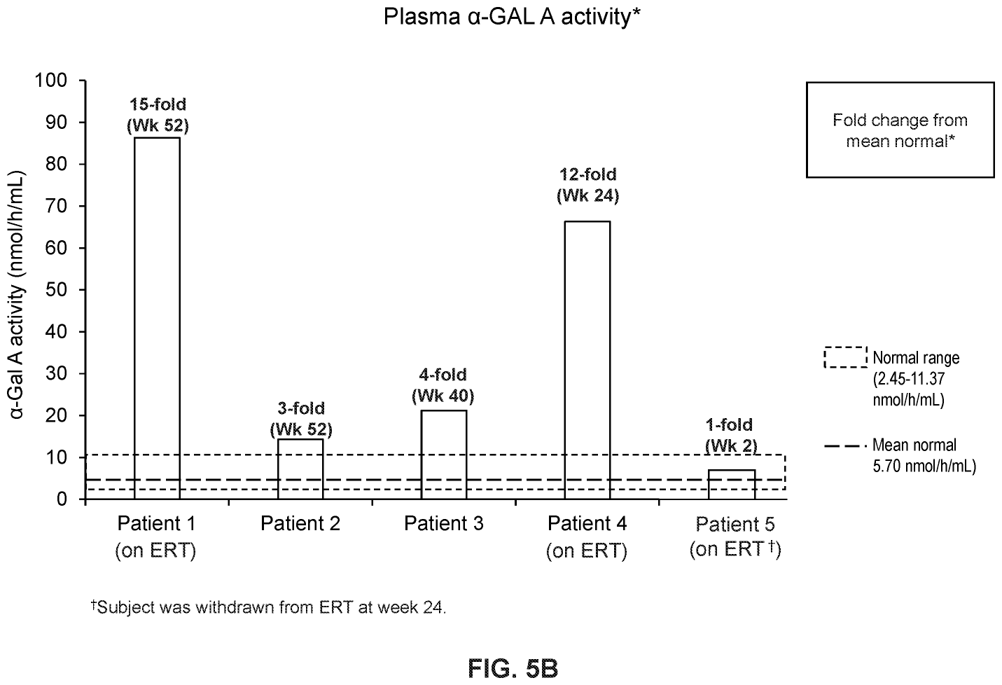

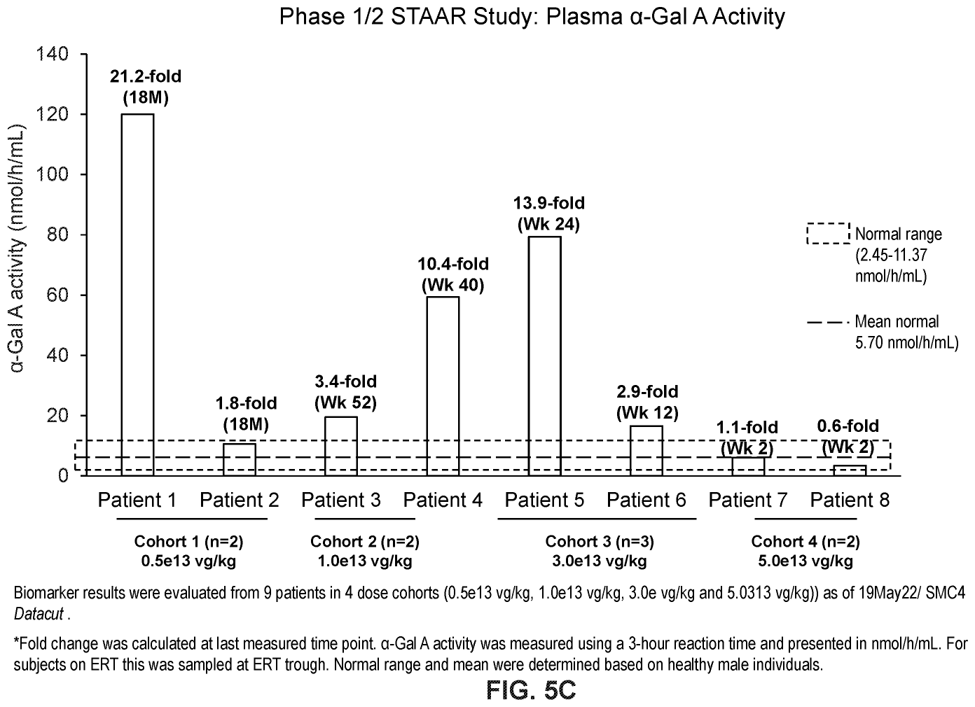

FIG. 5C shows that all subjects with available data by the data cutoff exhibit above normal levels of α-Gal activity.

Biomarker results were evaluated from the 8 subjects across 4 dose cohorts (0.5e13 vg/kg, 1.0e13 vg/kg, 3.0e13 vg/kg and 5.0e13 vg/kg) as of the cutoff date. Fold change was calculated at last measured time point. α-Gal A activity was measured using a 3-hour reaction time and presented in nmol/hr/mL. For patients 1, 4, 5, 6 and 7 this was sampled at ERT trough. Normal range and mean were determined based on healthy male individuals. FIG. 5C shows that elevated α-Gal A activity was observed through the data cutoff for subjects 1-8: up to 18 months for the first 2 subjects treated. α-Gal A activity is within normal at week 2 for subjects 7 and 8. Subjects 1 and 4 were withdrawn from ERT.

Activity levels of up to 21-fold above mean normal have been observed.

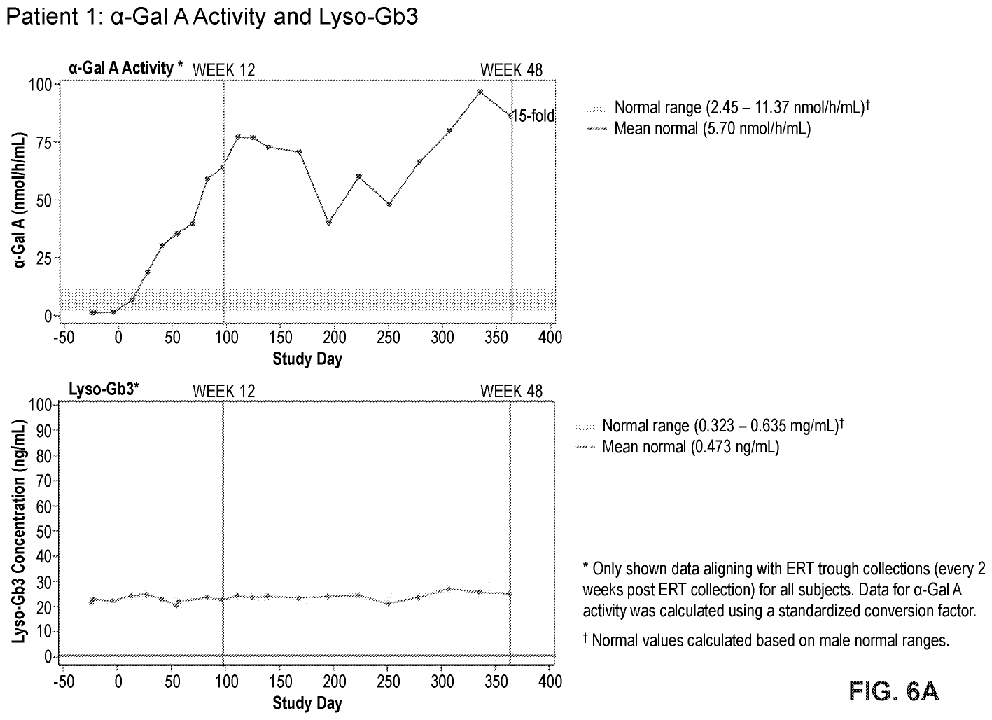

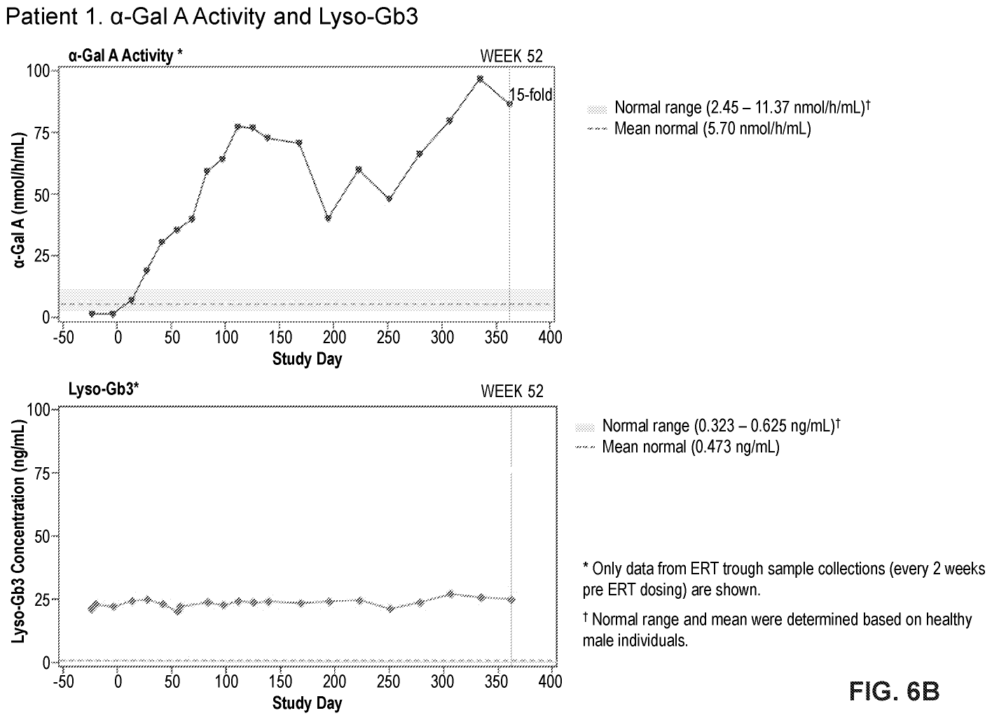

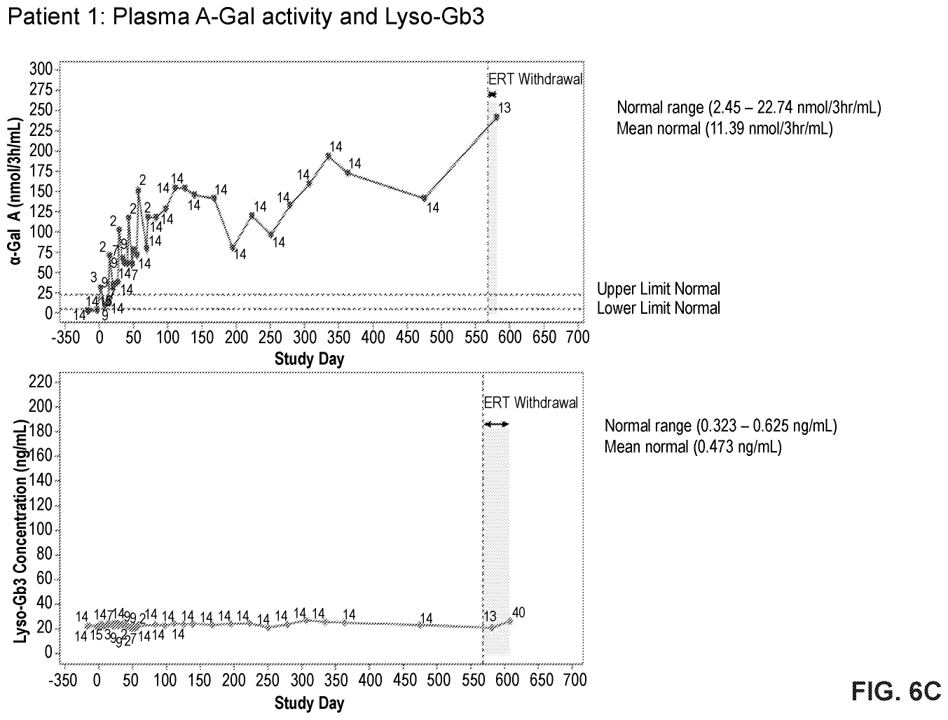

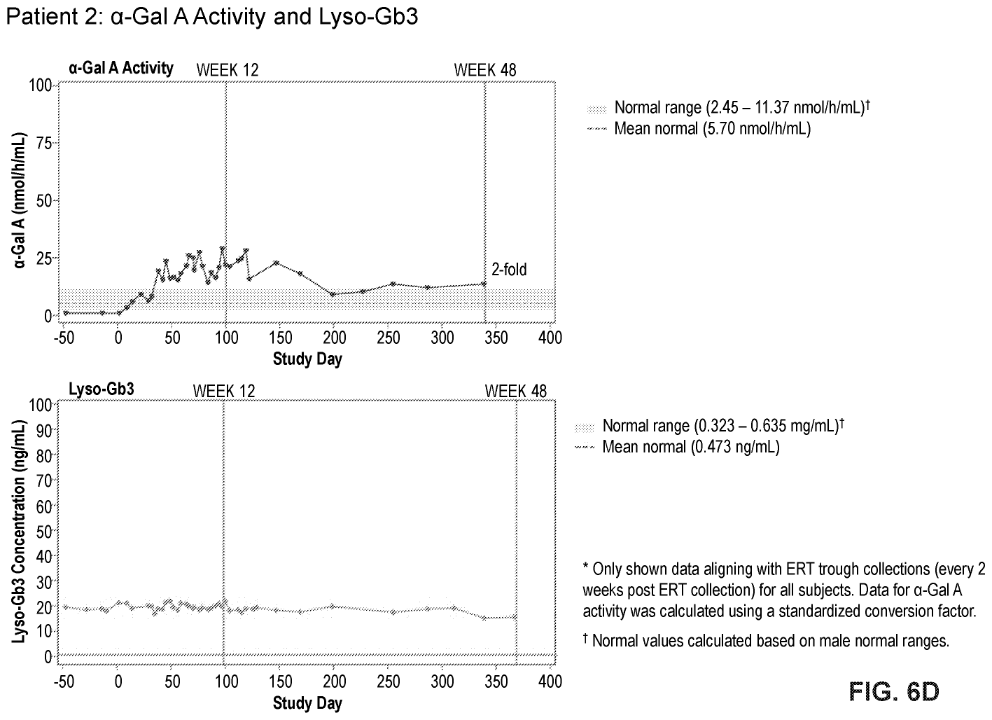

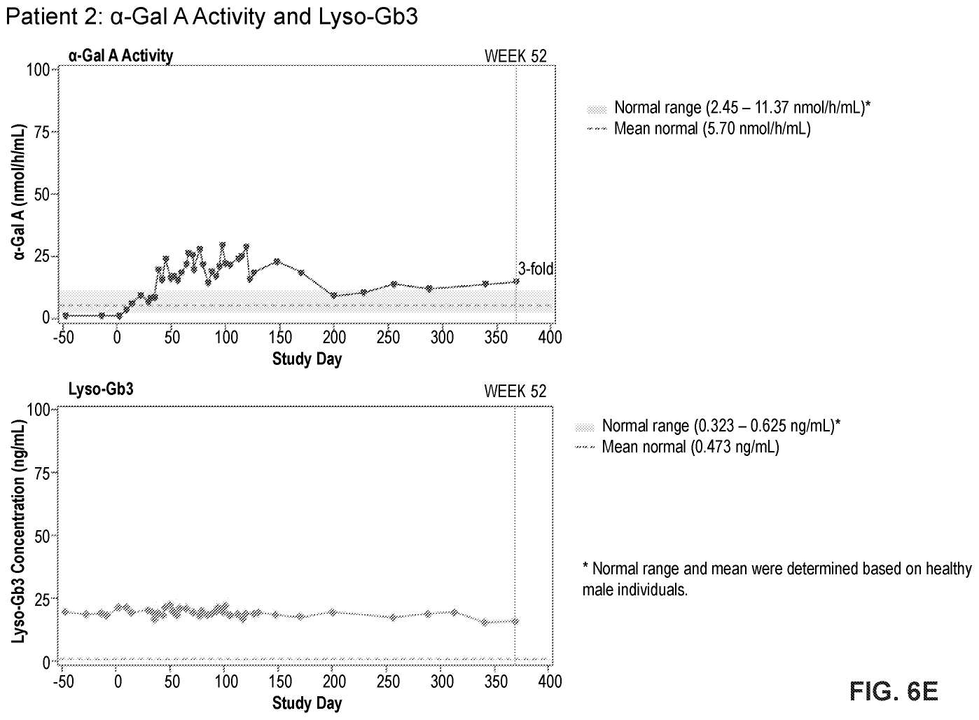

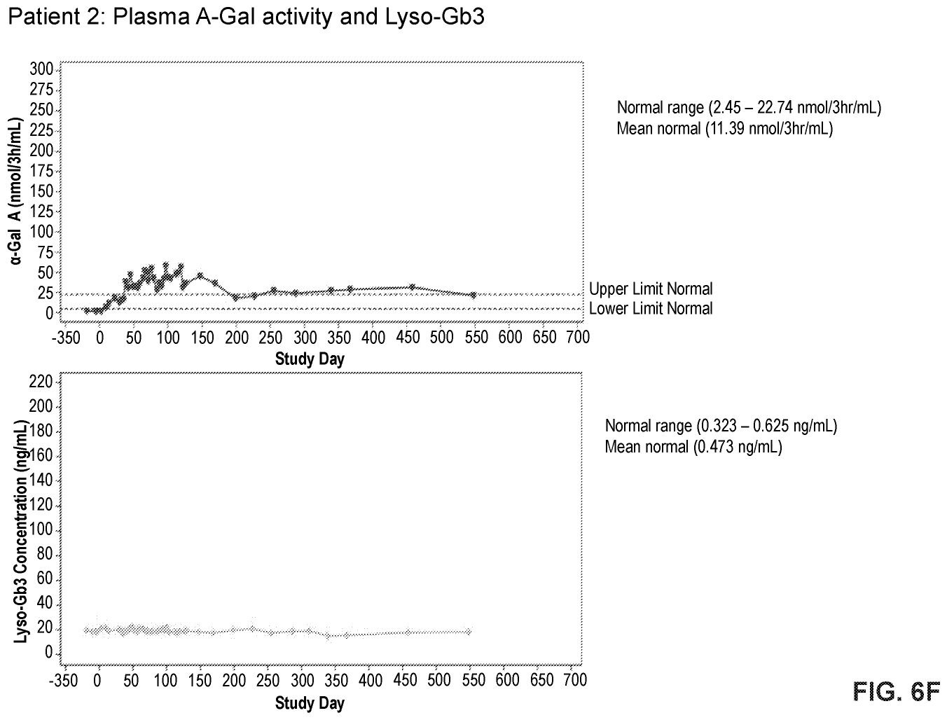

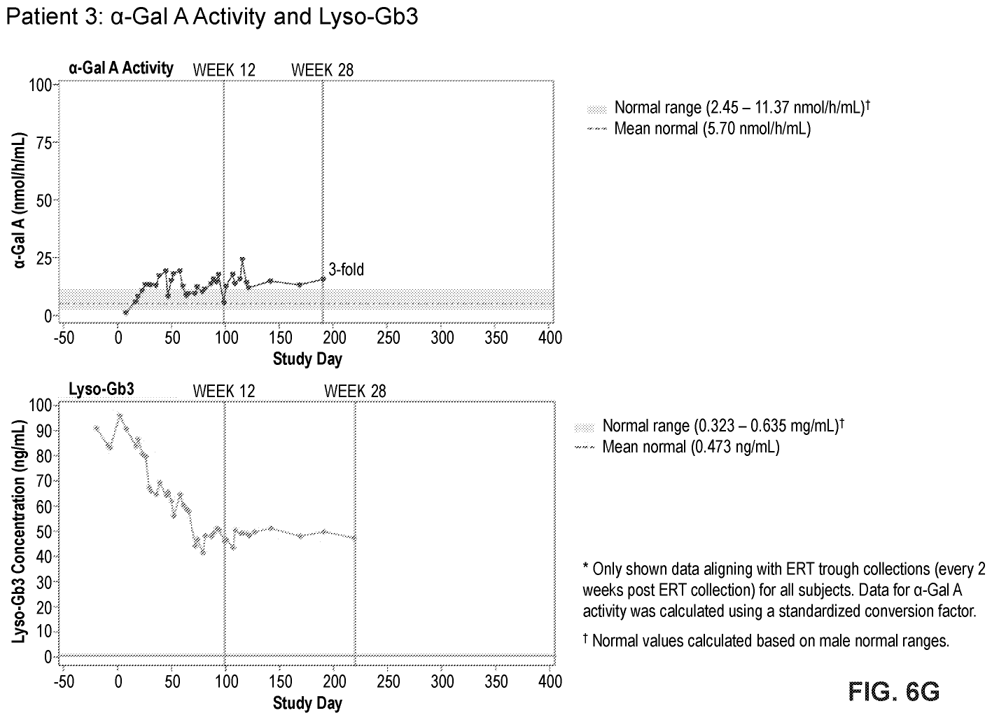

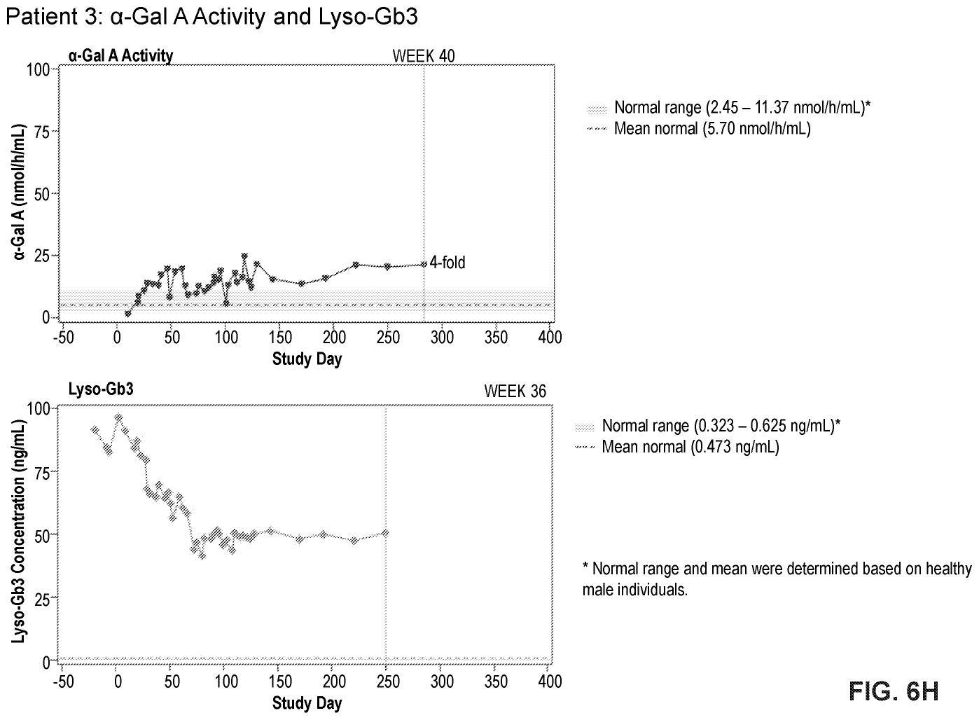

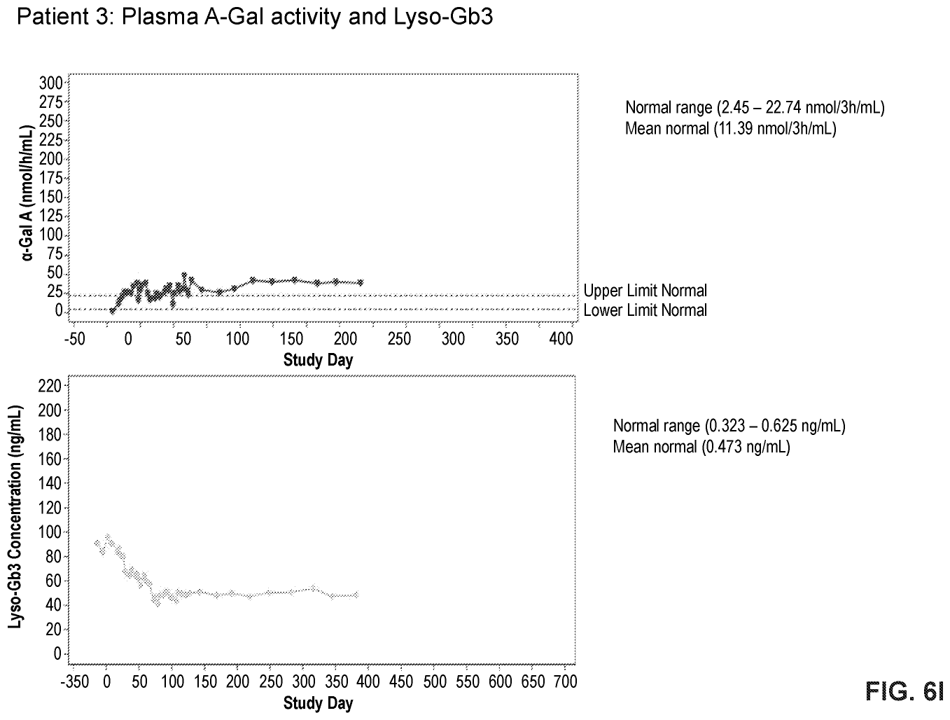

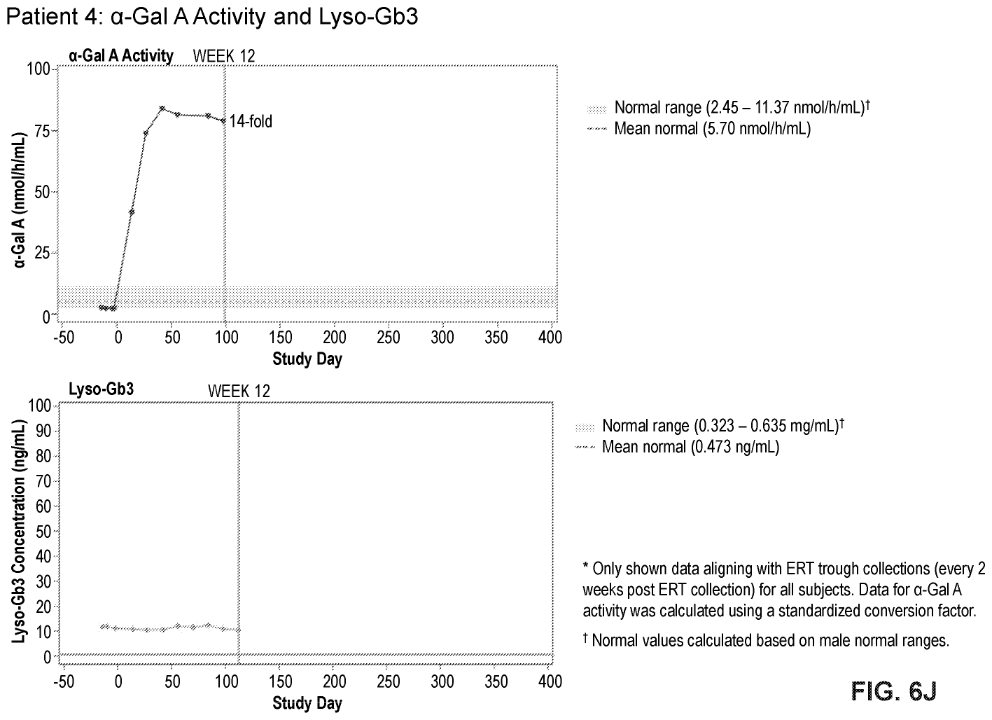

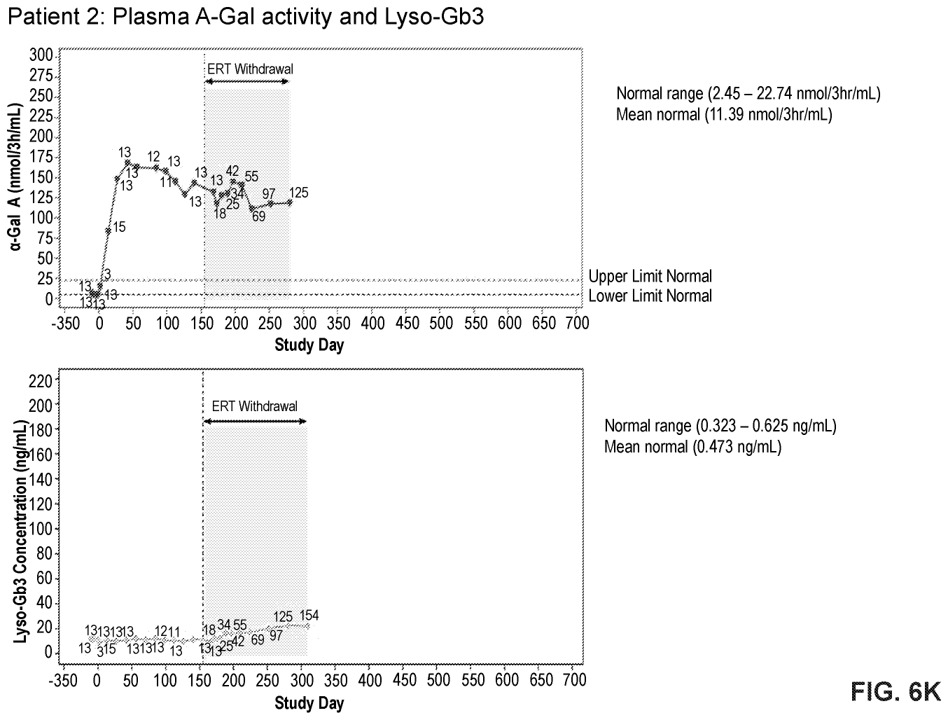

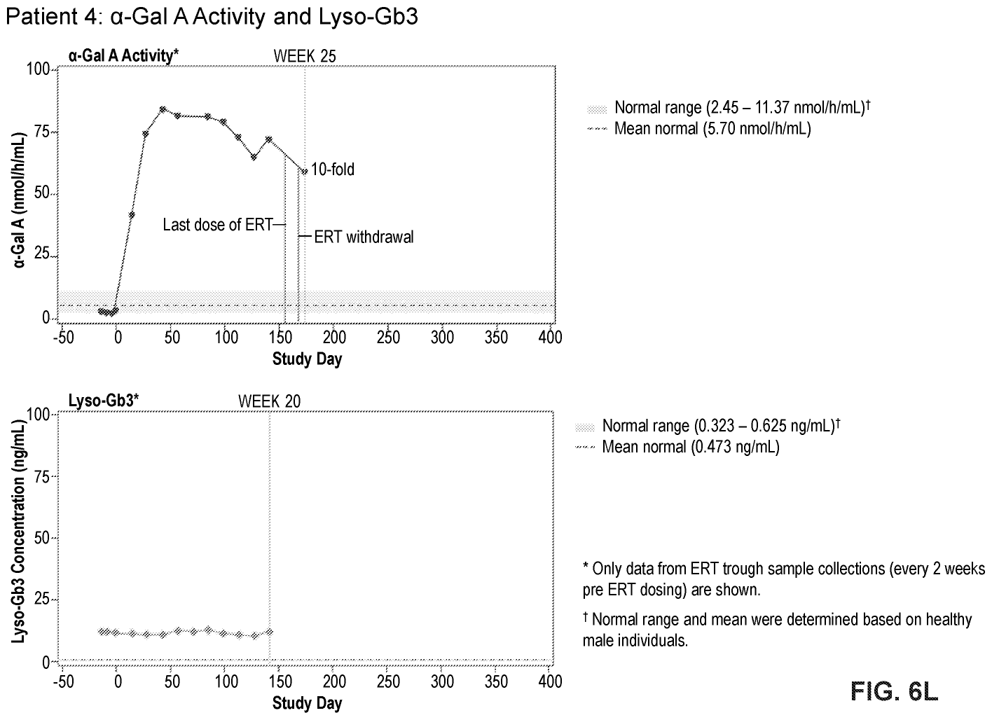

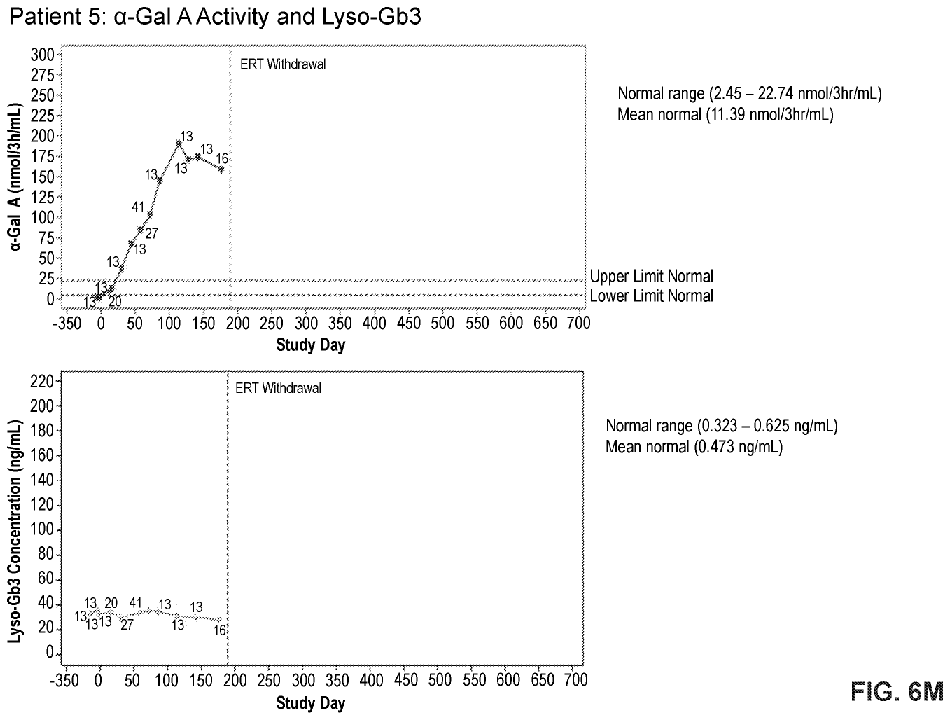

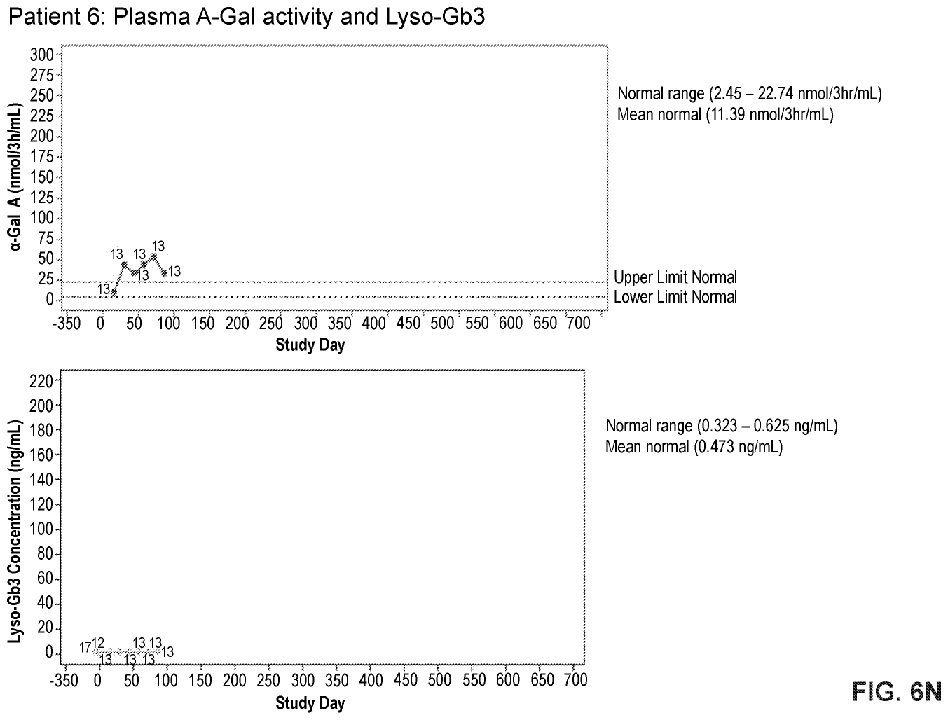

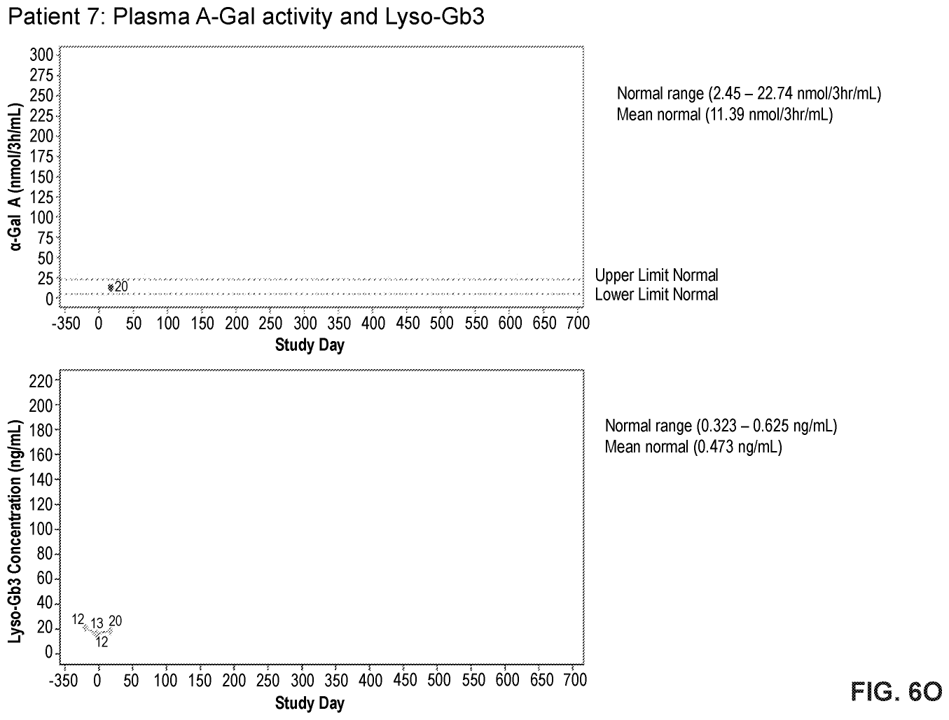

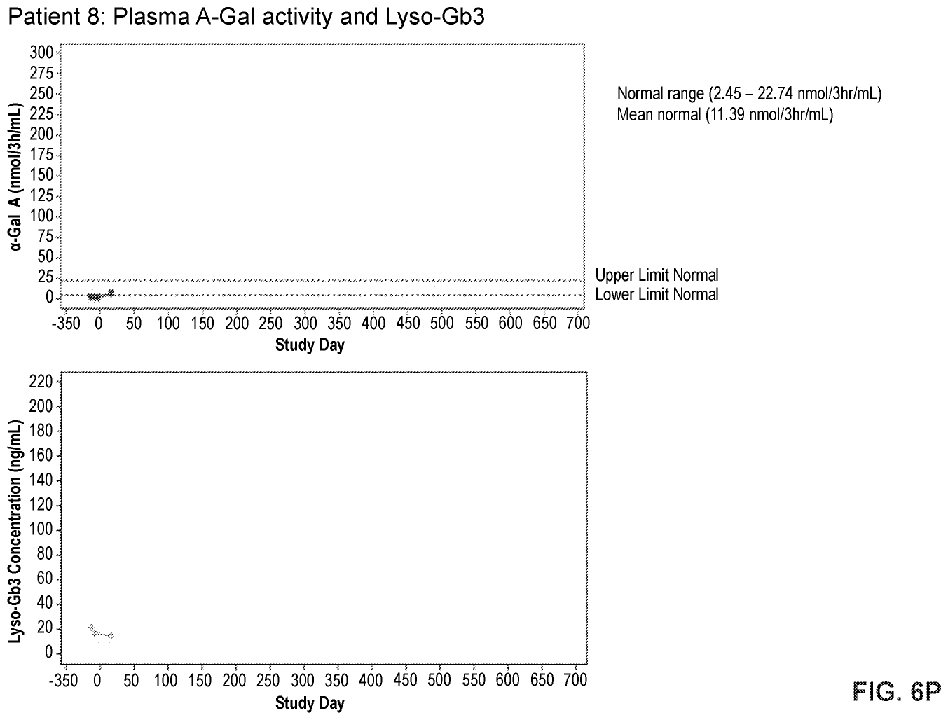

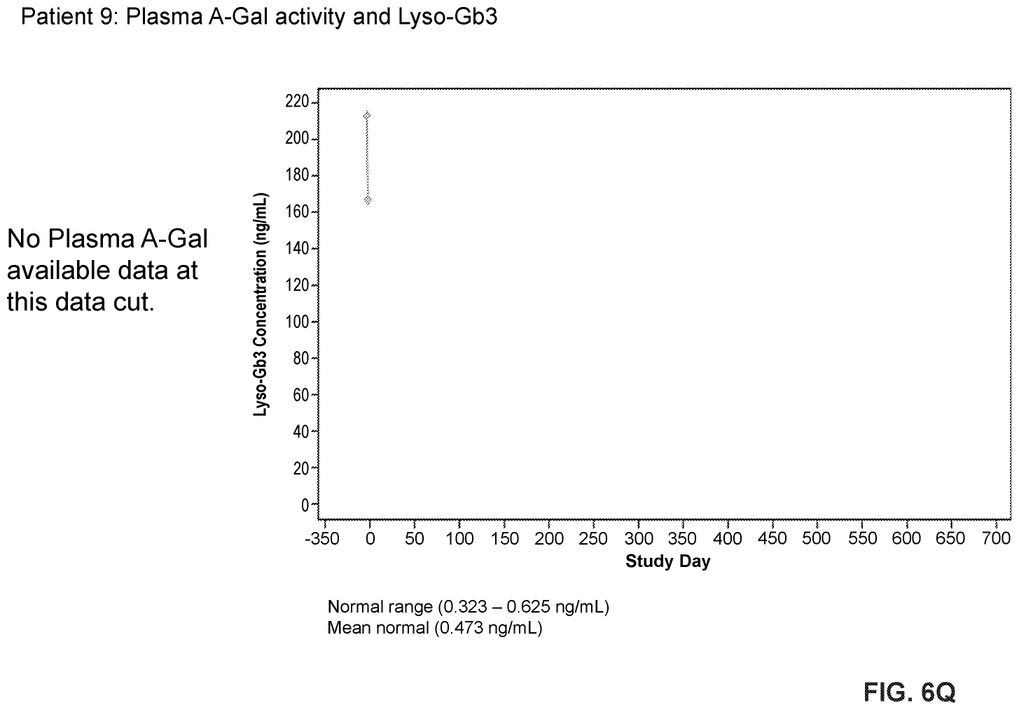

FIG. 6A shows that patient 1 exhibited above normal level of α-Gal activity that was sustained for 1 year to end of study. Lyso-Gb3 remained within 10% of baseline up to the end of study. FIG. 6B shows that patient 1 exhibited above normal level of α-Gal activity that was sustained for 48 weeks. FIG. 6C shows that patient 1 exhibited above normal level of α-Gal activity that was sustained for 18 months. Left ventricular hypertrophy (drop concentric) increased in run-in and stabilized following 1 year of treatment on MRI). Low baseline levels of plasma lyso-Gb3 remained steady over time. Patient 1 reported improvements in leg edema and ability to sweat. Patient 1 has rolled over into long-term follow up (follow up every 3 months for additional 4 years).

FIG. 6D shows that patient 2 exhibited above normal levels of α-Gal activity that sustained through 48 weeks. FIG. 6E shows that patient 2 exhibited above normal levels of α-Gal activity that was sustained for 1 year. FIG. 6F shows that patient 2 exhibited at or above normal levels of α-Gal activity that was sustained for 18 months. Patient's baseline mild biventricular dilation improved by MRI at 1 year. Low baseline levels of plasma lyso-Gb3 remained steady over time. Patient 2 reported improvement in ability to sweat. Patient 2 has rolled over into long-term follow up (follow up every 3 months for additional 4 years).

FIG. 6G shows that patient 3 exhibited above normal levels of α-Gal activity that was sustained up to the last measured point at week 28. Patient 2 also showed ˜40% reduction in lyso-Gb3 from baseline to week 28. Decline in lyso-Gb3 was observed during 12 weeks after AAV-001 administration. FIG. 6H shows that patient 3 exhibited above normal levels of α-Gal activity that was sustained up to the last measured point at week 40. FIG. 6I shows that patient 3 exhibited above normal levels of α-Gal activity that was sustained up to the last measured point at week 52. Patient's cardiac MRI normal at baseline and 24 weeks. Patient's plasma lyso-Gb3 levels were elevated at baseline. Patient 3 showed ˜40% reduction in plasma lyso-Gb3 from baseline within 10 weeks after dosing, which was maintained through week 52. Patient 3 reported improvement in ability to sweat.

FIG. 6J shows that patient 4 exhibited above normal levels of α-Gal activity that sustained through 12 weeks. FIG. 6K shows that patient 4 exhibited above normal levels of α-Gal activity that sustained through 25 weeks. FIG. 6L shows that patient 4 exhibited above normal levels of α-Gal activity that sustained through 40 weeks. Patient 4 presented with mild LVH at baseline. Patient 4 was withdrawn from ERT at week 24 (based on 2-week dosing frequency).

FIG. 6M shows that patient 5 exhibited above normal levels of α-Gal activity that sustained through 24 weeks. Patient 5 presented with mild LVH at baseline. FIG. 6N shows that patient 6 exhibited above normal levels of α-Gal activity that sustained through 12 weeks. Patient 6 presented with mild LVH at baseline. FIG. 6O shows that patient 7 baseline and dosing data of α-Gal. Patient 7 presented with mild to moderate LVH at baseline. FIG. 6P shows that patient 8 baseline and dosing data of α-Gal. Patient 8 presented with normal cardiac MRI at baseline. FIG. 6Q shows that patient 9 has no α-Gal data at the moment of this datacut. Patient 9 presented with moderate LVH at baseline.

What is claimed is:

1. A method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg.

2. A method of treating Fabry disease or ameliorating one or more symptoms associated with Fabry disease in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg.

3. A method of reducing the amount of glycosphingolipids in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α galactosidase A (α-Gal A) transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

4. A method of reducing the amount of glycosphingolipids in a human subject in need thereof, the method comprising administering to the subject an adeno-associated virus (AAV) expression vector comprising an α galactosidase A (α-Gal A) expression cassette, which comprises an α galactosidase A (α-Gal A) transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the reduced amount of glycosphingolipids is relative to the amount of glycosphingolipids in the subject prior to the administration.

5. A method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an apolipoprotein E (APOE) enhancer operably linked to an alpha 1-antitrypsin (hAAT) promoter, a human hemoglobin beta (HBB)-IGG intron, a sequence encoding a signal peptide, an α-Gal A transgene encoding the at least one α-Gal A protein, and a bovine growth hormone poly A signal sequence, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

6. A method of increasing an α galactosidase A (α-Gal A) protein activity in a human subject in need thereof, the method comprising administering to the subject an adeno-associated virus (AAV) expression vector comprising an α galactosidase A (α-Gal A) expression cassette, which comprises an α-Gal A transgene encoding the at least one α-Gal A protein, wherein the α-GalA transgene comprises the nucleotide sequence as set forth in SEQ ID NO: 5, wherein the AAV expression vector is administered at a dose of about 5×10¹² vector genomes per kilogram of body weight (vg/kg) to about 5×10¹³ vg/kg, wherein the increased α-Gal A protein activity is relative to the α-Gal A protein activity in the subject prior to the administration.

7. The method of claim any one of claims 3-6, wherein the subject has Fabry disease.

8. The method of any one of claims 1 to 7, wherein the α-Gal A expression cassette further comprises a mutated Woodchuck Hepatitis Virus (WHV) Posttranscriptional Regulatory Element (WPRE) sequence.

9. The method of claim 8, wherein the mutated WPRE sequence comprises a mut6 mutated WPRE sequence.

10. The method of any one of claims 2, 4, and 6, wherein the α-Gal A expression cassette further comprises an apolipoprotein E (APOE) enhancer operably linked to an alpha 1-antitrypsin (hAAT) promoter, a human hemoglobin beta (HBB)-IGG intron, a sequence encoding a signal peptide, and a bovine growth hormone poly A signal sequence.

11. The method of any one of claims 1, 3, and 5, wherein the transgene comprises a wild-type α-Gal A sequence or a codon-optimized α-Gal A sequence.

12. The method of any one of claims 1, 3, 5, 10, and 11, wherein the signal peptide is an α-GalA signal peptide.

13. The method of claim 8 or 9, wherein the enhancer comprises the nucleotide sequence as set forth in SEQ ID NO: 2, the promotor comprises the nucleotide sequence as set forth in SEQ ID NO: 3, the intron comprises the nucleotide sequence as set forth in SEQ ID NO: 4, the α-GalA transgene comprises the nucleotide sequence as set forth in SEQ ID NO: 5, the mutated WPRE sequence comprises the nucleotide sequence as set forth in SEQ ID NO: 6, and the poly A signal sequence comprises the nucleotide sequence as set forth in SEQ ID NO: 7.

14. The method of claim 8 or 9, wherein the enhancer comprises the nucleotide sequence as set forth in SEQ ID NO: 2, the promotor comprises the nucleotide sequence as set forth in SEQ ID NO: 3, the intron comprises the nucleotide sequence as set forth in SEQ ID NO: 4, the mutated WPRE sequence comprises the nucleotide sequence as set forth in SEQ ID NO: 6, and the poly A signal sequence comprises the nucleotide sequence as set forth in SEQ ID NO: 7.

15. The method of any one of claims 1 to 14, wherein the α-Gal A expression cassette comprises the nucleotide sequence as set forth in SEQ ID NO: 9.

16. The method of any one of claims 1 to 15, wherein the AAV expression vector serotype is AAV2/6.

17. The method of any one of claims 1 to 16, wherein the subject has one or more of the following symptoms: globotriaosylceramide (Gb3) levels above normal, globotriaosylsphingosine (lyso-Gb3) levels above normal, renal disease, cardiac disease, anhidrosis, acroparesthesia, angiokeratoma, gastrointestinal (GI) tract pain, corneal and lenticular opacities, or cerebrovascular disease.

18. The method of claim 17, wherein angiokeratoma is periumbilical angiokeratoma.

19. The method of any one of claims 5 to 18, wherein the subject has the α-GalA protein activity of less than about 5%.

20. The method of claim 19, wherein the α-GalA protein activity is measured in the subject's plasma and/or leukocytes.

21. The method of any one of claims 1 to 20, wherein the subject is a male subject.

22. The method of any one of claims 1 to 20, wherein the subject is a female subject.

23. The method of any one of claims 1 to 22, wherein the subject has an α-GalA gene mutation that is indicative of Fabry disease.

24. The method of claim 19, wherein the subject is an enzyme replacement therapy (ERT) naïve subject.

25. The method of any one of claims 1 to 24, wherein the subject has pre-existing anti-α-GalA antibodies prior to the administering as determined by an enzyme-linked immunosorbent assay (ELISA).

26. The method of any one of claims 1 to 25, wherein the subject is an anti-α-GalA neutralizing antibody positive subject when a biological sample of the subject is analyzed, wherein the anti-α-GalA neutralizing antibody positive subject has a biological sample having greater than about 9.6% inhibition of α-Galactosidase A activity as measured by an anti-α-GalA neutralizing antibody assay.

27. The method of any one of claims 1 to 26, wherein the α-Gal A protein expressed from the transgene decreases the amount of glycosphingolipids in the subject by at least about 2 fold as compared to the amount of glycosphingolipids in the subject prior to the administration.

28. The method of any one of claims 1 to 26, wherein the α-Gal A protein expressed from the transgene decreases the amount of glycosphingolipids in the subject by about 10 percent (%), about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% as compared to the amount of glycosphingolipids in the subject prior to the administration.

29. The method of any one of claims 1, 2, and 5 to 26, wherein the α-Gal A protein expressed from the transgene maintains the amount of glycosphingolipids in the subject at the same level as prior to the administration.

30. The method of any one of claims 3, 4, and 7 to 29, wherein glycosphingolipids comprise globotriaosylceramide (Gb3), globotriaosylsphingosine (lyso-Gb3), galabiosylceramide, or any combination thereof.

31. The method of claim 30, wherein Gb3 and/or lyso-Gb3 levels are measured in the subject's plasma, tissue, and/or urine.

32. The method of any one of claims 1 to 28 and 30 to 31, wherein the α-Gal A protein expressed from the transgene decreases the amount of glycosphingolipids in one or more of plasma, liver, heart, kidney, urine, skin, or spleen.

33. The method of any one of claims 5 to 32, wherein the α-Gal A protein activity in the subject is between about 0-fold higher to about 2-fold higher, between about 2-fold higher to about 5-fold higher, between about 5-fold higher to about 10-fold higher, between about 10-fold higher to about 20-fold higher, between about 20-fold higher to about 30-fold higher, between about 30-fold higher to about 40-fold higher, between about 30-fold higher to about 40-fold higher, between about 40-fold higher to about 50-fold higher, between about 50-fold higher to about 60-fold higher, between about 60-fold higher to about 70-fold higher, between about 70-fold higher to about 80-fold higher, between about 80-fold higher to about 90-fold higher, between about 90-fold higher to about 100-fold higher, between about 100-fold higher to about 200-fold higher, between about 200-fold higher to about 300-fold higher, between about 300-fold higher to about 400-fold higher, between about 400-fold higher to about 500-fold higher than the mean normal α-Gal A protein activity compared to the α-Gal A protein activity in the subject prior to the administration.

34. The method of claim 33, wherein the α-Gal A protein activity in the subject is between about 0-fold higher to about 2-fold higher, between about 2-fold higher, about 3-fold higher, about 4-fold higher, about 5-fold higher, about 6-fold higher, about 7-fold higher, about 8-fold higher, about 9-fold higher, about 10-fold higher, about 11-fold higher, about 12-fold higher, about 13-fold higher, about 14-fold higher, about 15-fold higher, about 16-fold higher, about 17-fold higher, about 18-fold higher, about 19-fold higher, about 20-fold higher, about 21-fold higher, about 22-fold higher, about 23-fold higher, about 24-fold higher, about 25-fold higher, about 26-fold higher, about 27-fold higher, about 28-fold higher, about 29-fold higher, about 30-fold higher, about 31-fold higher, about 32-fold higher, about 33-fold higher, about 34-fold higher, about 35-fold higher, about 36-fold higher, about 37-fold higher, about 38-fold higher, about 39-fold higher, about 40-fold higher, about 41-fold higher, about 42-fold higher, about 43-fold higher, about 44-fold higher, about 45-fold higher, about 46-fold higher, about 47-fold higher, about 48-fold higher, about 49-fold higher, about 50-fold higher, about 51-fold higher, about 52-fold higher, about 53-fold higher, about 54-fold higher, about 55-fold higher, about 56-fold higher, about 57-fold higher, about 58-fold higher, about 59-fold higher, about 60-fold higher, about 61-fold higher, about 62-fold higher, about 63-fold higher, about 64-fold higher, about 65-fold higher, about 66-fold higher, about 67-fold higher, about 68-fold higher, about 69-fold higher, about 70-fold higher, about 71-fold higher, about 72-fold higher, about 73-fold higher, about 74-fold higher, about 75-fold higher, about 76-fold higher, about 77-fold higher, about 78-fold higher, about 79-fold higher, about 80-fold higher, about 81-fold higher, about 82-fold higher, about 83-fold higher, about 84-fold higher, about 85-fold higher, about 86-fold higher, about 87-fold higher, about 88-fold higher, about 89-fold higher, about 90-fold higher, about 91-fold higher, about 92-fold higher, about 93-fold higher, about 94-fold higher, about 95-fold higher, about 96-fold higher, about 97-fold higher, about 98-fold higher, about 99-fold higher, or about 100-fold higher than the mean normal α-Gal A protein activity compared to the α-Gal A protein activity in the subject prior to the administration.

35. The method of any one of claims 1 to 34, wherein the levels of the α-Gal A protein expressed from the transgene are measured in one or more of the subject's plasma, serum, whole blood, dried blood spot, leukocytes, or other blood components.

36. The method of any one of claims 1 to 35, wherein the α-Gal A protein expressed from the transgene is active in the subject's kidneys, liver, skin and heart.

37. The method of any one of claims 1 to 36, wherein the AAV expression vector is administered parenterally.

38. The method of any one of claims 1 to 37, wherein the AAV expression vector is administered intravenously.

39. The method of any one of claims 1 to 38, wherein the AAV expression vector is administered in a pharmaceutically acceptable carrier.

40. The method of claim 39, wherein the pharmaceutically acceptable carrier comprises phosphate buffered saline containing CaCl₂, MgCl₂, NaCl, Sucrose, and Kolliphor (Poloxamer) P 188.

41. The method of any one of claims 1 to 40, wherein only one dose of the AAV expression vector is administered to the subject.

42. The method of any one of claims 1 to 41, wherein the AAV expression vector is administered at a dose of about 5×10¹² vg/kg.

43. The method of any one of claims 1 to 41, wherein the AAV expression vector is administered at a dose of about 1×10¹³ vg/kg.

44. The method of any one of claims 1 to 41, wherein the AAV expression vector is administered at a dose of about 3×10¹³ vg/kg.

45. The method of any one of claims 1 to 41, wherein the AAV expression vector is administered at a dose of about 5×10¹³ vg/kg.

46. The method of any one of claims 1 to 45, wherein the subject is administered an immunosuppressant prior to and/or during administration of the AAV expression vector.

47. The method of claim 46, wherein the immunosuppressant comprises prednisone.

48. The method of any one of claims 1 to 45, wherein the subject is not administered an immunosuppressant prior to and/or during administration of the AAV expression vector.

49. The method of any one of claims 1 to 48, wherein the subject is not administered a preconditioning treatment prior to the administration of the AAV expression vector.

50. The method of any one of claims 1 to 50, wherein the expression of the at least one α-Gal A protein is sustained for at least 3 months, at least 4 months, at least 5 months, at least 6 months, at least 7 months, at least 8 months, at least 9 months, at least 10 months, at least 11 months, at least 12 months, at least 13 months, at least 14 months, at least 15 months, at least 16 months, at least 17 months, at least 18 months, at least 19 months, at least 20 months, at least 21 months, at least 22 months, at least 23 months, or at least 24 months.

51. The method of any one of claims 1 to 50, wherein an Estimated Glomerular filtration rate (eGFR) in ml/min/1.73 m² is measured in the subject after the administering using the Chronic Kidney Disease Epidemiology Collaboration (CKD-EPI) equation.

52. The method of claim 51, wherein the rate of annual eGFR decline is lower than in a comparable untreated subject with Fabry disease.

53. The method of any one of claims 1 to 52, wherein an Ejection Fraction (EF) is measured in the subject as stroke volume (SV)/left ventricular volumes at end-diastole (LVEDV) after the administering.

54. The method of claim 53, wherein the rate of annual EF decline is lower than in a comparable untreated subject with Fabry disease.

55. The method of any one of claims 1 to 54, wherein a Global Longitudinal Strain (GLS) is measured in the subject by a two-dimensional (2D) strain echocardiography or cardiac magnetic resonance imaging (cardiac MRI or CMR) after the administering.

56. The method of claim 55, wherein the annual shortening progression the contractibility of the muscles of the heart is lower than in a comparable untreated subject with Fabry disease.

57. The method of any one of claims 1 to 56, wherein a Left Ventricular Mass Index (LVMI) is measured as left ventricular mass (LVM)/body surface area in the subject after the administering.

58. The method of claim 57, wherein the annual LVMI increase is lower than in a comparable untreated subject with Fabry disease.

59. The method of any one of claims 1 to 58, wherein there is an improvement in one or more audiologic symptoms in the subject after the administration.

60. The method of claim 59, wherein one or more audiologic symptoms are tinnitus, vertigo, or progressive hearing loss.

61. The method of any one of claims 1 to 60, wherein the subject had a positive change in the level of perspiration from anhidrosis to hypohidrosis or normal hidrosis.

62. The method of any one of claims 1 to 61, wherein the subject has been administered with an enzyme replacement therapy (ERT) for Fabry disease prior to the administering (“pre-treatment”).

63. The method of claim 62, wherein the enzyme replacement therapy for the pre-treatment comprises a recombinant α-Galactosidase A (GLA) protein or a gene expressing GAL.

64. The method of claim 62 or 63, wherein the enzyme replacement therapy for the pre-treatment comprises administering galafold, AVR-RD-01, FLT-190, pegunigalsidase alfa, 4D-310, or any combination thereof.

65. The method of any one of claims 62 to 64, wherein the enzyme replacement therapy for the pre-treatment comprises a recombinant α-Galactosidase A (GLA) protein in combination with an active site-specific chaperone (ASSC) for the GLA.

66. The method of claim 65, wherein the ASSC is 1-deoxygalactonojirimycin.

67. The method of claim 62 or 63, wherein the enzyme replacement therapy for the pre-treatment comprises agalsidase alpha and/or beta or a gene expressing agalsidase alpha and/or beta.

68. The method of claim 67, wherein the enzyme replacement therapy for the pre-treatment comprises fabrazyme, Replagal, PRX-102, or any combination thereof.

69. The method of claim 63 or 68, wherein the enzyme replacement therapy for the pre-treatment comprises a gene therapy.

70. The method of claim 69, wherein the gene therapy comprises a vector encoding the enzyme.

71. The method of claim 69, wherein the gene therapy comprises administering AVR-RD-01, FLT-190, pegunigalsidase alfa, 4D-310, or any combination thereof.

72. The method of claim 70, wherein the vector comprises an mRNA encoding a human GLA protein or agalsidase alpha and/or beta.

73. The method of claim 70, wherein the vector is a viral vector.

74. The method of claim 73, wherein the viral vector comprises an adeno-associated virus (AAV) vector or a lentiviral vector.

75. The method of claim 69, wherein the gene therapy is delivered by a lipid nanoparticle.

76. The method of any one of claims 1 to 61, wherein the subject has been administered with a non-enzyme replacement therapy for Fabry disease prior to the administering (“pre-treatment”).

77. The method of claim 76, wherein the pre-treatment therapy for Fabry disease comprises lucerastat, venglustat, apabetalone, or any combination thereof.

78. The method of any one of claims 1, 2, and 7 to 77, wherein Fabry disease is type 1 classic phenotype or type 2 later-onset phenotype.

79. The method of any one of claims 1 to 78, wherein the α-Gal A expression cassette is flanked on each end by inverted terminal repeats (ITRs).

80. The method of claim 79, wherein the ITRs are derived from adeno-associated virus type 2 (AAV2).

81. The method of any one of claims 1 to 80, wherein the AAV expression vector further comprises the α-Gal A expression cassette packaged with capsid derived from adeno-associated virus.

82. The method of claim 81, wherein the α-Gal A expression cassette is packaged with capsid derived from adeno-associated virus type 6 (AAV6).
